(19) United States
(12) Patent Application Publication
(10) Pub. No.: US 2011/0257208 A1
(43) Pub. Date: Oct. 20, 2011

(54) COMPOUNDS USEFUL AS FAAH MODULATORS AND USES THEREOF

(76) Inventors: Matthew Duncton, San Bruno, CA (US); Donogh John Roger O'mahony, San Mateo, CA (US); Matthew Cox, San Francisco, CA (US)

(21) Appl. No.: 13/130,051

(22) PCT Filed: Nov. 17, 2009
(86) PCT No.: PCT/US09/64757

§ 371 Date: Jul. 1, 2011

Related U.S. Application Data
(60) Provisional application No. 61/116,292, filed on Nov. 19, 2008.
(60) Provisional application No. 61/139,600, filed on Dec. 20, 2008.

(51) Int. Cl. A61K 31/519; A61P 25/00; A61P 25/24; A61P 25/18; A61P 11/00; A61P 17/06; A61P 9/10; A61P 9/12; A61P 21/00; A61P 27/06; A61P 37/02; A61P 13/00; A61P 1/08; A61P 35/00; A61P 25/22; C07D 487/04

(52) U.S. Cl. 514/265.1; 544/280

(57) ABSTRACT

Compounds are disclosed that have formula I: where A, B, L¹, W, Y, R¹, and R³ are as defined herein. The compounds and pharmaceutical compositions thereof are useful for the prevention and treatment of a variety of conditions in mammals including humans, including by way of non-limiting example, pain, anxiety, depression, inflammation, cognitive disorders, weight and eating disorders, Parkinson's disease, Alzheimer's disease, spasticity, addiction, glaucoma, and others.

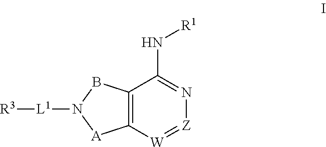

### FIELD OF THE INVENTION

This invention relates to novel compounds that are capable of modulating FAAH (fatty acid amide hydrolase) activity, and to pharmaceutical compositions containing such compounds. The invention further relates to preparation of such compounds. This invention also relates to methods for preventing and/or treating conditions that are causally related to aberrant FAAH activity or can be alleviated by modulating FAAH activity, such as pain, sleep disorders, anxiety and depression disorders, weight and eating disorders, Parkinson's disease, addiction, spasticity, inflammatory disorders, glaucoma, hypertension, or other disorders.

### BACKGROUND OF THE INVENTION

Fatty acid amide hydrolase (FAAH) is an integral membrane protein that degrades fatty acid primary amides and ethanolamides, a class of endogenous, lipid signaling molecules. FAAH has been shown to be relevant to the in vivo degradation of anandamide (AEA), oleamide, N-palmitoyl ethanolamide (PEA), N-oleoyl ethanolamide (OEA) and N-acyl taurines. These molecules act through a number of pathways and regulate diverse physiological behaviors including anxiety, pain, satiety, cognition and sleep (M. K. McKinney and B. J. Cravatt, (2005) Annu. Rev. Biochem. 74, 411-432).

The distribution of FAAH in the CNS suggests that it degrades neuromodulating fatty acid amides at their sites of action and is intimately involved in their regulation (E. A. Thomas, et al., (1997) J. Neurosci. Res. 50, 1047-1052). The creation of the FAAH KO (knockout) mouse confirmed that degradation of anandamide (AEA), oleamide, N-palmitoyl ethanolamide (PEA), N-oleoyl ethanolamide (OEA) and N-acyl taurines is regulated by FAAH in mice brains, as elevated levels of these molecules were observed in the absence of FAAH (B. F. Cravatt, et al., (2001), Proc. Natl. Acad. Sci. USA 98, 9371-9376; A. H. Lichtman, et al., (2002), J. Pharmacol. Exp. Ther. 302, 73-79; and A. Saghatelian, et al., (2004), Biochem. 43, 14332-14339).

As a number of small molecule inhibitors of FAAH have also been identified (M. Mor, et al., (2004), J. Med Chem. 47, 4998-5008; and D. L. Boger, et al., (2005) J. Med. Chem. 48, 1849-1856) both genetic (FAAH KO) and chemical tools were available to study the effect of FAAH inhibition in vivo. Additionally, some preliminary evidence exists regarding the existence of endogenous substances capable of activating FAAH (M. Maccarrone, et al., (2004) Mol. Hum. Reprod. 10, 215-221), this suggests that FAAH activation may be possible in vivo. Based on the fact that anandamide is both a CB1 receptor and a CB2 receptor agonist, amongst other biological roles, most of the investigations conducted focused on areas known to be affected by the activity of these two receptors. Interestingly, FAAH inhibition did not produce the side effects common to all CB1 receptor agonists: catalepsy, hypothermia, hypomotility and hyperphagia. The consequences of FAAH inhibition by a small molecule inhibitor were tested in animal models of anxiety and depression (S. Kathuria, et al., (2003), Nat. Med. 9, 76-81; and G. Gobbi, et al., (2005) Proc. Natl. Acad. Sci. USA 102, 18620-18625). Results indicated that FAAH inhibition led to increased levels of anandamide in the brain and concurrent anxiolytic and anti-depressant effects.

Several studies using both FAAH KO mice and small molecules inhibitors also demonstrated that FAAH inhibition led to analgesia in multiple animal models of pain (L. Chang, et al., (2006) Br. J. Pharmacol. 148, 102-113; A. Jayamanne, et al., (2005) Br. J. Pharmacol. 147, 281-288; M. D. Jhaveri, et al., (2006) J. Neurosci. 26, 13318-13327; and B. F. Cravatt, et al., (2001), Proc. Natl. Acad. Sci. USA 98, 9371-9376). Furthermore, a FAAH inhibitor was found to possess anti-inflammatory activity as it reduced carrageenan-induced hind paw inflammation in pentobarbital treated mice (S. Holt, et al., (2005) Br. J. Pharmacol. 146, 467-476). Recently, a link between FAAH inhibition and a potential treatment for Parkinson's disease was established (A. C. Kreitzer and R. C. Malenka, (2007) Nature, 445, 643-647). Endocannabinoids were revealed to play a role in the indirect pathway eCB-LTD (Long Term Depression) that is hypothesized to be absent in patients afflicted with the disease. Co-administration of a dopamine D2 receptor agonist and a FAAH inhibitor markedly reduced the motor deficits observed in two animal models of Parkinson's disease. These data clearly demonstrate that FAAH activity can be modulated by small molecules in vivo and that modulation of FAAH activity in vivo has clear potential therapeutic effects for several indications such as anxiety, depression, pain, Parkinson's disease and inflammation (M. K. McKinney and B. J. Cravatt, (2005) Annu. Rev. Biochem. 74, 411-432).

Accordingly, a need therefore exists for the development of agents, i.e. compounds, that are effective as modulators of FAAH activity, and it is toward the fulfillment of that need, that the present invention is directed.

### SUMMARY OF THE INVENTION

Compounds, and pharmaceutical compositions thereof, having potency and selectivity in the prevention and treatment of conditions that have been associated with neurological and inflammatory disorders and dysfunctions are described herein.

In particular, compounds, pharmaceutical compositions and methods provided are used to treat, prevent or ameliorate a range of conditions in mammals such as, but not limited to, pain of various genesis or etiology, for example acute, chronic, inflammatory and neuropathic pain, dental pain, dysmenorrhea and headache (such as migraine, cluster headache and tension headache). In some embodiments, the compounds, pharmaceutical compositions and methods provided are useful for the treatment of inflammatory pain and associated hyperalgesia and allodynia. In some embodiments, the compounds, pharmaceutical compositions and methods provided are useful for the treatment of neuropathic pain and associated hyperalgesis and allodynia (e.g. trigeminal or herpetic neuralgia, diabetic neuropathy, causalgia, sympathetically maintained pain and deafferentation syndromes such as brachial plexus avulsion). In some embodiments, the compounds, pharmaceutical compositions and methods provided are useful as anti-inflammatory agents for the treatment of arthritis, and as agents to treat Parkinson's Disease, Alzheimer's Disease, asthma, myocardial infarction, neurodegenerative disorders, spasticity, inflammatory bowel disease and autoimmune disorders, fever, atherosclerosis and cardiovascular diseases, renal disorders, bone disorders, obesity, eating disorders, nausea, emesis, cancer, memory disorders, schizophrenia, epilepsy, sleeping disorders, cognitive disorders, depression, anxiety, high blood pressure, addiction, glaucoma and lipid disorders.

Accordingly, in one aspect, compounds are provided that have formula I:

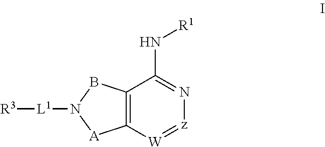

wherein
each A and B is independently CR^{2a}R^{2b};
W and Z are independently N or CR⁴;
L¹ is a single bond or substituted or unsubstituted C₁-C₅ alkylene, —CO—, —NHC(O)—, —OC(O)—, —SO—, or S(O)₂—;
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof; and stereoisomers, isotopic variants and tautomers thereof;
provided that the compound is other than
N-[5-(1,1-dimethylethyl)-2-methylphenyl]-6,7-dihydro-2-[4-(4-methoxyphenyl)-1-piperazinyl]-6-(methylsulfonyl)-5H-pyrrolo[3,4-d]pyrimidin-4-amine;
4-[[5-(1,1-dimethylethyl)-2-methylphenyl]amino]-5,7-dihydro-2-[4-(4-methoxyphenyl)-1-piperazinyl]-6H-Pyrrolo[3,4-d]pyrimidine-6-carboxylic acid, 1,1-dimethylethyl ester;
2-chloro-4-[[3-(dimethylamino)-5-methoxyphenyl]amino]-5,7-dihydro-6H-pyrrolo[3,4-d]pyrimidine-6-carboxylic acid, 9H-fluoren-9-ylmethyl ester;
2-chloro-5,7-dihydro-4-[(5-methyl-1H-pyrazol-3-yl)amino]-6H-pyrrolo[3,4-d]pyrimidine-6-carboxylic acid, 9H-fluoren-9-ylmethyl ester; and
N-(2,3-dihydro-1H-inden-2-yl)-6,7-dihydro-2-(methylthio)-6-(phenylmethyl)-5H-pyrrolo[3,4-d]pyrimidin-4-amine.

In another aspect, compounds are provided that have formula I:

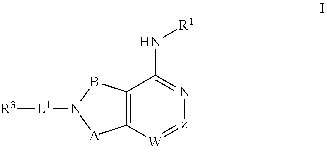

wherein
each A and B is independently CR^{2a}R^{2b};
W and Z are independently N or CR⁴;
L¹ is —CO—, —NHC(O)—, —OC(O)—, —SO—, or S(O)₂—;
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof; and stereoisomers, isotopic variants and tautomers thereof;
provided that the compound is other than
N-[5-(1,1-dimethylethyl)-2-methylphenyl]-6,7-dihydro-2-[4-(4-methoxyphenyl)-1-piperazinyl]-6-(methylsulfonyl)-5H-pyrrolo[3,4-d]pyrimidin-4-amine;
4-[[5-(1,1-dimethylethyl)-2-methylphenyl]amino]-5,7-dihydro-2-[4-(4-methoxyphenyl)-1-piperazinyl]-6H-Pyrrolo[3,4-d]pyrimidine-6-carboxylic acid, 1,1-dimethylethyl ester;
2-chloro-4-[[3-(dimethylamino)-5-methoxyphenyl]amino]-5,7-dihydro-6H-pyrrolo[3,4-d]pyrimidine-6-carboxylic acid, 9H-fluoren-9-ylmethyl ester; and
2-chloro-5,7-dihydro-4-[(5-methyl-1H-pyrazol-3-yl)amino]-6H-pyrrolo[3,4-d]pyrimidine-6-carboxylic acid, 9H-fluoren-9-ylmethyl ester.

In yet another aspect, compounds are provided that have formula I:

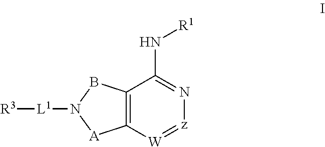

wherein
each A and B is independently CR^{2a}R^{2b};
W and Z are independently N or CR⁴;
L¹ is a single bond or substituted or unsubstituted C₁-C₅ alkylene;
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof;
and stereoisomers, isotopic variants and tautomers thereof;
provided that the compound is other than N-(2,3-dihydro-1H-inden-2-yl)-6,7-dihydro-2-(methylthio)-6-(phenylmethyl)-5H-pyrrolo[3,4-d]pyrimidin-4-amine.

In another aspect, pharmaceutical compositions are provided comprising a compound of the invention, and a pharmaceutical carrier, excipient or diluent. The pharmaceutical composition can comprise one or more of the compounds described herein. In a further embodiment, the pharmaceutical compositions of the invention can comprise a compound in combination with one or more other compounds and/or compositions having a like therapeutic effect.

It will be understood that compounds of the present invention useful in the pharmaceutical compositions and treatment methods disclosed herein, can be pharmaceutically acceptable as prepared and used.

In another aspect, methods are provided for preventing, treating or ameliorating a condition from among those listed herein, and particularly, such condition as may be associated with, e.g., arthritis, asthma, myocardial infarction, lipid disorders, cognitive disorders, anxiety, schizophrenia, depression, memory dysfunctions such as Alzheimers disease, inflammatory bowel disease and autoimmune disorders, which method comprises administering to a mammal in need thereof an amount of one or more of the compounds as provided herein, or pharmaceutical composition thereof, effective to prevent, treat or ameliorate the condition.

In yet another aspect, methods are provided for preventing, treating or ameliorating a variety of disease states, including the diseases associated with pain, sleep disorders, anxiety and depression disorders, weight and eating disorders, addiction, spasticity, and glaucoma, by administration of a compound such as those provided herein.

In a further aspect, methods are provided for preventing, treating or ameliorating a neurodegenerative disease or disorder in a mammal A neurodegenerative disease or disorder can, for example, be Parkinson's disease, Alzheimer's disease and multiple sclerosis; diseases and disorders which are mediated by or result in neuroinflammation such as, for example, encephalitis; centrally-mediated neuropsychiatric diseases and disorders such as, for example, depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders; itch/pruritus such as, for example, psoriasis; obesity; lipid disorders; cancer; and renal disorders For example, the compounds of the invention might be used in conjunction with a dopamine D2 receptor agonist to treat Parkinson's disease, in accordance with the recent findings of Malenka (A. C. Kreitzer and R. C. Malenka, (2007) Nature, 445, 643-647), that FAAH inhibition in conjunction with dopamine administration promises an effective treatment of the disease. Typically, the methods comprise administering an effective condition-treating or condition-preventing amount of one or more of the compounds as provided herein, or pharmaceutical composition thereof, to the mammal in need thereof.

In addition to the methods of treatment set forth above, the present invention extends to the use of any of the compounds of the invention for the preparation of medicaments that may be administered for such treatments, as well as to such compounds for the treatments disclosed and specified.

In additional aspects, methods are provided for synthesizing the compounds described herein, with representative synthetic protocols and pathways described below.

Accordingly, it is a principal object of the invention to provide a novel series of compounds, which can modify any aberrant activity of FAAH and thus may have the ability to treat certain of the conditions in which FAAH is believed to play a role.

A still further object of the invention is to provide pharmaceutical compositions that are effective in the treatment or prevention of a variety of disease states, including the diseases associated with pain, sleep disorders, anxiety and depression disorders, weight and eating disorders, addiction, spasticity, intraocular pressure or other disorders.

A still further object of the invention is to provide a method for the treatment of the disease states recited above, by the administration of a therapeutically effective amount of the compounds of the invention, and/or the pharmaceutical compositions of the invention.

A yet further object of the invention is to provide formulations for the treatment of the diseases as aforesaid, by the combination of at least one of the compounds of the invention, a pharmaceutical composition of the invention, combinations thereof with other compounds and compositions having a like therapeutic effect.

Other objects and advantages will become apparent to those skilled in the art from a consideration of the ensuing detailed description.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

‘Acyl’ or ‘Alkanoyl’ refers to a radical —C(O)R²⁰, where R²⁰ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein. Representative examples include, but are not limited to, formyl, acetyl, cyclohexylcarbonyl, cyclohexylmethylcarbonyl, benzoyl and benzylcarbonyl. Exemplary ‘acyl’ groups are —C(O)H, —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocycloalkyl), wherein t is an integer from 0 to 4.

‘Substituted Acyl’ or ‘Substituted Alkanoyl’ refers to a radical —C(O)R²¹, wherein R²¹ is independently
C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Acylamino’ refers to a radical-NR²²C(O)R²³, where R²² is hydrogen, C₁-C₈ alkyl, C₃-C₁₀ cycloalkyl, 4-10 membered heterocycloalkyl, C₆-C₁₀ aryl, arylalkyl, 5-10 membered heteroaryl or heteroarylalkyl and R²³ is hydrogen, C₁-C₈ alkyl, C₃-C₁₀ cycloalkyl, 4-10 membered heterocycloalkyl, C₆-C₁₀ aryl, arylalkyl, 5-10 membered heteroaryl or heteroarylalkyl, as defined herein. Exemplary ‘acylamino’ include, but are not limited to, formylamino, acetylamino, cyclohexylcarbonylamino, cyclohexylmethyl-carbonylamino, benzoylamino and benzylcarbonylamino. Particular exemplary ‘acylamino’ groups are —NR²⁴C(O)—C₁-C₈ alkyl, —NR²⁴C(O)—(CH₂)ₜ(C₆-C₁₀ aryl), —NR²⁴C(O)—(CH₂)ₜ(5-10 membered heteroaryl), —NR²⁴C(O)—(CH₂)ₜ(C₃-C₁₀ cycloalkyl), and —NR²⁴C(O)—(CH₂)ₜ(4-10 membered heterocycloalkyl), wherein t is an integer from 0 to 4, and each R²⁴ independently represents H or C₁-C₈ alkyl.

‘Substituted Acylamino’ refers to a radical —NR²⁵C(O)R²⁶, wherein:

R²⁵ is independently
H, C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and

R²⁶ is independently
H, C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl;

provided at least one of R²⁵ and R²⁶ is other than H.

‘Acyloxy’ refers to a radical —OC(O)R²⁷, where R²⁷ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein. Representative examples include, but are not limited to, formyl, acetyl, cyclohexylcarbonyl, cyclohexylmethylcarbonyl, benzoyl and benzylcarbonyl. Exemplary ‘acyl’ groups are —C(O)H, —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocycloalkyl), wherein t is an integer from 0 to 4.

‘Substituted Acyloxy’ refers to a radical —OC(O)R²⁸, wherein R²⁸ is independently
C₁-C₈ alkyl, substituted with halo or hydroxy; or
C₃-C₁₀ cycloalkyl, 4-10 membered heterocycloalkyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Alkoxy’ refers to the group —OR²⁹ where R²⁹ is C₁-C₈ alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

‘Substituted alkoxy’ refers to an alkoxy group substituted with one or more of those groups recited in the definition of “substituted” herein, and particularly refers to an alkoxy group having 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocycloalkyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups are —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are OCF₃, OCH₂CF₃, OCH₂Ph, OCH₂-cyclopropyl, OCH₂CH₂OH, and OCH₂CH₂NMe₂.

‘Alkoxycarbonyl’ refers to a radical —C(O)—OR³⁰ where R³⁰ represents an C₁-C₈ alkyl, C₃-C₁₀ cycloalkyl, C₃-C₁₀ cycloalkylalkyl, 4-10 membered heterocycloalkylalkyl, aralkyl, or 5-10 membered heteroarylalkyl as defined herein. Exemplary “alkoxycarbonyl” groups are C(O)O—C₁-C₈ alkyl, —C(O)O—(CH₂)ₜ(C₆-C₁₀ aryl), —C(O)O—(CH₂)ₜ(5-10 membered heteroaryl), —C(O)O—(CH₂)ₜ(C₃-C₁₀ cycloalkyl), and —C(O)O—(CH₂)ₜ(4-10 membered heterocycloalkyl), wherein t is an integer from 1 to 4.

‘Substituted Alkoxycarbonyl’ refers to a radical —C(O)—OR³¹ where R³¹ represents:
C₁-C₈ alkyl, C₃-C₁₀ cycloalkyl, C₃-C₁₀ cycloalkylalkyl, or 4-10 membered heterocycloalkylalkyl, each of which is substituted with halo, substituted or unsubstituted amino, or hydroxy; or
C₆-C₁₀ aralkyl, or 5-10 membered heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl.

‘Aryloxycarbonyl’ refers to a radical —C(O)—OR³² where R³² represents an C₆-C₁₀ aryl, as defined herein. Exemplary “aryloxycarbonyl” groups is —C(O)O—(C₆-C₁₀ aryl).

‘Substituted Aryloxycarbonyl’ refers to a radical —C(O)—OR³³ where R³³ represents
C₆-C₁₀ aryl, substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl.

‘Heteroaryloxycarbonyl’ refers to a radical —C(O)—OR³⁴ where R³⁴ represents a 5-10 membered heteroaryl, as defined herein. An exemplary “aryloxycarbonyl” group is —C(O)O-(5-10 membered heteroaryl).

‘Substituted Heteroaryloxycarbonyl’ refers to a radical —C(O)—OR³⁵ where R³⁵ represents:
5-10 membered heteroaryl, substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl.

‘Alkoxycarbonylamino’ refers to the group —NR³⁶C(O)OR³⁷, where R³⁶ is hydrogen, C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein, and R³⁷ is C₁-C₈ alkyl, C₃-C₁₀ cycloalkyl, C₃-C₁₀ cycloalkylmethyl, 4-10 membered heterocycloalkyl, aryl, arylalkyl, 5-10 membered heteroaryl or heteroarylalkyl as defined herein.

‘Alkyl’ means straight or branched aliphatic hydrocarbon having 1 to 20 carbon atoms. Particular alkyl has 1 to 12 carbon atoms. More particular is lower alkyl which has 1 to 6 carbon atoms. A further particular group has 1 to 4 carbon atoms. Exemplary straight chained groups include methyl, ethyl n-propyl, and n-butyl. Branched means that one or more lower alkyl groups such as methyl, ethyl, propyl or butyl is attached to a linear alkyl chain, exemplary branched chain groups include isopropyl, iso-butyl, t-butyl and isoamyl.

‘Substituted alkyl’ refers to an alkyl group as defined above substituted with one or more of those groups recited in the definition of “substituted” herein, and particularly refers to an alkyl group having 1 or more substituents, for instance from 1 to 5 substituents, and particularly from 1 to 3 substituents, in particular 1 substituent, selected from the group consisting of acyl, acylamino, acyloxy (—O-acyl or —OC(O)R²⁰), alkoxy, alkoxycarbonyl, alkoxycarbonylamino (—NR″-alkoxycarbonyl or —NH—C(O)—OR²⁷), amino, substituted amino, aminocarbonyl (carbamoyl or amido or —C(O)—NR″₂), aminocarbonylamino (—NR″—C(O)—NR″₂), aminocarbonyloxy (—O—C(O)—NR″₂), aminosulfonyl, sulfonylamino, aryl, aryloxy, azido, carboxyl, cyano, cycloalkyl, halogen, hydroxy, heteroaryl, nitro, thiol, —S-alkyl, —S-aryl, —S(O)-alkyl, —S(O)-aryl, —S(O)₂-alkyl, and —S(O)₂-aryl. In a particular embodiment ‘substituted alkyl’ refers to a C₁-C₈ alkyl group substituted with halo, cyano, nitro, trifluoromethyl, trifluoromethoxy, azido, —NR′″SO₂R″, —SO₂NR″R′″, —C(O)R″, —C(O)OR″, —OC(O)R″, —NR′″C(O)R″, —C(O)NR″R′″, —NR″R′″, or —(CR′″R″″)ₘOR′″; wherein each R″ is independently selected from H, C₁-C₈ alkyl, —(CH₂)ₜ(C₆-C₁₀ aryl), —(CH₂)ₜ(5-10 membered heteroaryl), —(CH₂)ₜ(C₃-C₁₀ cycloalkyl), and —(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Each of R′″ and R″″ independently represents H or C₁-C₈ alkyl.

‘Alkylene’ refers to divalent saturated alkene radical groups having 1 to 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

‘Substituted alkylene’ refers to those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, amino-carbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Alkenyl’ refers to monovalent olefinically unsaturated hydrocarbyl groups preferably having 2 to 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

‘Substituted alkenyl’ refers to those groups recited in the definition of ‘substituted’ herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Alkenylene’ refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

‘Alkynyl’ refers to acetylenically or alkynically unsaturated hydrocarbyl groups particularly having 2 to 11 carbon atoms, and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

‘Substituted alkynyl’ refers to those groups recited in the definition of “substituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Amino’ refers to the radical —NH₂.

‘Substituted amino’ refers to an amino group substituted with one or more of those groups recited in the definition of ‘substituted’ herein, and particularly refers to the group —N(R³⁸)₂ where each R³⁸ is independently selected from:
hydrogen, C₁-C₈ alkyl, C₆-C₁₀ aryl, 5-10 membered heteroaryl, 4-10 membered heterocycloalkyl, or C₃-C₁₀ cycloalkyl; or
C₁-C₈ alkyl, substituted with halo or hydroxy; or
—(CH₂)ₜ(C₆-C₁₀ aryl), —(CH₂)ₜ(5-10 membered heteroaryl), —(CH₂)ₜ(C₃-C₁₀ cycloalkyl) or —(CH₂)ₜ(4-10 membered heterocycloalkyl) wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or
both R³⁸ groups are joined to form an alkylene group.
   When both R³⁸ groups are hydrogen, —N(R³⁸)₂ is an amino group. Exemplary ‘substituted amino’ groups are —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocycloalkyl), wherein t is an integer from 0 to 4, each R³⁹ independently represents H or C₁-C₈ alkyl; and any alkyl groups present, may themselves be substituted by halo, substituted or unsubstituted amino, or hydroxy; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term “substituted amino” includes the groups alkylamino, substituted alkylamino, alkylarylamino, substituted alkylarylamino, arylamino, substituted arylamino, dialkylamino and substituted dialkylamino as defined below.

‘Alkylamino’ refers to the group —NHR⁴⁰, wherein R⁴⁰ is C₁-C₈ alkyl;

‘Substituted Alkylamino’ refers to the group —NHR⁴¹, wherein R⁴¹ is C₁-C₈ alkyl; and the alkyl group is substituted with halo, substituted or unsubstituted amino, hydroxy, C₃-C₁₀ cycloalkyl, 4-10 membered heterocycloalkyl, C₆-C₁₀ aryl, 5-10 membered heteroaryl, aralkyl or heteroaralkyl; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Alkylarylamino’ refers to the group —NR⁴²R⁴³, wherein R⁴² is aryl and R⁴³ is C₁-C₈ alkyl.

‘Substituted Alkylarylamino’ refers to the group —NR⁴⁴R⁴⁵, wherein R⁴⁴ is aryl and R⁴⁵ is C₁-C₈ alkyl; and the alkyl group is substituted with halo, substituted or unsubstituted amino, hydroxy, C₃-C₁₀ cycloalkyl, 4-10 membered heterocycloalkyl, C₆-C₁₀ aryl, 5-10 membered heteroaryl, aralkyl or heteroaralkyl; and any aryl, heteroaryl, cycloalkyl or heterocycloalkyl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Arylamino’ means a radical —NHR⁴⁶ where R⁴⁶ is selected from C₆-C₁₀ aryl and 5-10 membered heteroaryl as defined herein.

‘Substituted Arylamino’ refers to the group —NHR⁴⁷, wherein R⁴⁷ is independently selected from C₆-C₁₀ aryl and 5-10 membered heteroaryl; and any aryl or heteroaryl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Dialkylamino’ refers to the group —NR⁴⁸R⁴⁹, wherein each of R⁴⁸ and R⁴⁹ are independently selected from C₁-C₈ alkyl.

‘Substituted Dialkylamino’ refers to the group —NR⁵⁰R⁵¹, wherein each of R⁵⁹ and R⁵¹ are independently selected from C₁-C₈ alkyl; and at least one of the alkyl groups is independently substituted with halo, hydroxy, C₃-C₁₀ cycloalkyl, 4-10 membered heterocycloalkyl, C₆-C₁₀ aryl, 5-10 membered heteroaryl, aralkyl or heteroaralkyl; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁₋₄ haloalkyl, unsubstituted C₁-C₄ hydroxyalkyl, or unsubstituted C₁-C₄ haloalkoxy or hydroxy.

‘Diarylamino’ refers to the group —NR⁵²R⁵³, wherein each of R⁵² and R⁵³ are independently selected from C₆-C₁₀ aryl.

‘Aminosulfonyl’ or ‘Sulfonamide’ refers to the radical —S(O₂)NH₂.

‘Substituted aminosulfonyl’ or ‘substituted sulfonamide’ refers to a radical such as —S(O₂)N(R⁵⁴)₂ wherein each R⁵⁴⁸ is independently selected from:
H, C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy;
   provided that at least one R⁵⁴ is other than H.

Exemplary ‘substituted aminosulfonyl’ or ‘substituted sulfonamide’ groups are —S(O₂)N(R⁵⁵)—C₁-C₈ alkyl, —S(O₂)N(R⁵⁵)—(CH₂)ₜ(C₆-C₁₀ aryl), —S(O₂)N(R⁵⁵)—(CH₂)ₜ(5-10 membered heteroaryl), —S(O₂)N(R⁵⁵)—(CH₂)ₜ(C₃-C₁₀ cycloalkyl), and —S(O₂)N(R⁵⁵)—(CH₂)ₜ(4-10 membered heterocycloalkyl), wherein t is an integer from 0 to 4; each R⁵⁵ independently represents H or C₁-C₈ alkyl; and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Aralkyl’ or ‘arylalkyl’ refers to an alkyl group, as defined above, substituted with one or more aryl groups, as defined above. Particular aralkyl or arylalkyl groups are alkyl groups substituted with one aryl group.

‘Substituted Aralkyl’ or ‘substituted arylalkyl’ refers to an alkyl group, as defined above, substituted with one or more aryl groups; and at least one of the aryl groups present, may themselves be substituted by unsubstituted C₁-C₄ alkyl, halo, cyano, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Aryl’ refers to a monovalent aromatic hydrocarbon group derived by the removal of one hydrogen atom from a single carbon atom of a parent aromatic ring system. In particular aryl refers to an aromatic ring structure, mono-cyclic or poly-cyclic that includes from 5 to 12 ring members, more usually 6 to 10. Where the aryl group is a monocyclic ring system it preferentially contains 6 carbon atoms. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene and trinaphthalene. Particularly aryl groups include phenyl, naphthyl, indenyl, and tetrahydronaphthyl.

‘Substituted Aryl’ refers to an aryl group substituted with one or more of those groups recited in the definition of ‘substituted’ herein, and particularly refers to an aryl group that may optionally be substituted with 1 or more substituents, for instance from 1 to 5 substituents, particularly 1 to 3 substituents, in particular 1 substituent. Particularly, ‘Substituted Aryl’ refers to an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

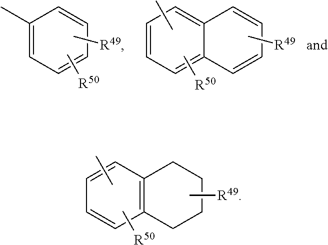

In these formulae one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocycloalkyl, alkanoyl, C₁-C₈ alkoxy, heteroaryloxy, alkylamino, arylamino, heteroarylamino, NR⁵⁸COR⁵⁹, NR⁵⁸SOR⁵⁹, NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O or S. R⁶⁰, and R⁶¹ are independently hydrogen, C₁-C₈ alkyl, C₁-C₄ haloalkyl, C₃-C₁₀ cycloalkyl, 4-10 membered heterocycloalkyl, C₆-C₁₀ aryl, substituted aryl, 5-10 membered heteroaryl.

‘Fused Aryl’ refers to an aryl having two of its ring carbon in common with a second aryl ring or with an aliphatic ring.

‘Arylalkyloxy’ refers to an —O-alkylaryl radical where alkylaryl is as defined herein.

‘Substituted Arylalkyloxy’ refers to an —O-alkylaryl radical where alkylaryl is as defined herein; and any aryl groups present, may themselves be substituted by unsubstituted C₁-C₄ alkyl, halo, cyano, unsubstituted C₁-C₄ alkoxy, unsubstituted C₁₋₄ haloalkyl, unsubstituted C₁-C₄ hydroxyalkyl, or unsubstituted C₁-C₄ haloalkoxy or hydroxy.

‘Azido’ refers to the radical —N₃.

‘Carbamoyl or amido’ refers to the radical —C(O)NH₂.

‘Substituted Carbamoyl or substituted amido’ refers to the radical —C(O)N(R⁶²)₂ wherein each R⁶² is independently
H, C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy;
   provided that at least one R⁶² is other than H.
   Exemplary ‘Substituted Carbamoyl’ groups are —C(O)NR⁶⁴—C₁-C₈ alkyl, —C(O)NR⁶⁴—(CH₂)ₜ(C₆-C₁₀ aryl), —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶⁴—(CH₂)ₜ(4-10 membered heterocycloalkyl), wherein t is an integer from 0 to 4, each R⁶⁴ independently represents H or C₁-C₈ alkyl and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Carboxy’ refers to the radical —C(O)OH.

‘Cycloalkyl’ refers to cyclic non-aromatic hydrocarbyl groups having from 3 to 10 carbon atoms. Such cycloalkyl groups include, by way of example, single ring structures such as cyclopropyl, cyclobutyl, cyclopentyl, and cyclooctyl.

‘Substituted cycloalkyl’ refers to a cycloalkyl group as defined above substituted with one or more of those groups recited in the definition of ‘substituted’ herein, and particularly refers to a cycloalkyl group having 1 or more substituents, for instance from 1 to 5 substituents, and particularly from 1 to 3 substituents, in particular 1 substituent

‘Cyano’ refers to the radical —CN.

‘Halo’ or ‘halogen’ refers to fluoro (F), chloro (Cl), bromo (Br) and iodo (I). Particular halo groups are either fluoro or chloro.

‘Hetero’ when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, e.g. cycloheteroalkenyl, and the like having from 1 to 5, and particularly from 1 to 3 heteroatoms.

‘Heteroaryl’ means an aromatic ring structure, mono-cyclic or polycyclic, that includes one or more heteroatoms and 5 to 12 ring members, more usually 5 to 10 ring members. The heteroaryl group can be, for example, a five membered or six membered monocyclic ring or a bicyclic structure formed from fused five and six membered rings or two fused six membered rings or, by way of a further example, two fused five membered rings. Each ring may contain up to four heteroatoms typically selected from nitrogen, sulphur and oxygen. Typically the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Examples of five membered monocyclic heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups. Examples of six membered monocyclic heteroaryl groups include but are not limited to pyridine, pyrazine, pyridazine, pyrimidine and triazine. Particular examples of bicyclic heteroaryl groups containing a five membered ring fused to another five membered ring include but are not limited to imidazothiazole and imidazoimidazole. Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, isoindolone, indolizine, indoline, isoindoline, purine (e.g., adenine, guanine), indazole, pyrazolopyrimidine, triazolopyrimidine, benzodioxole and pyrazolopyridine groups. Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups. Particular heteroaryl groups are those derived from thiophene, pyrrole, benzothiophene, benzofuran, indole, pyridine, quinoline, imidazole, oxazole and pyrazine.

Examples of representative heteroaryls include the following:

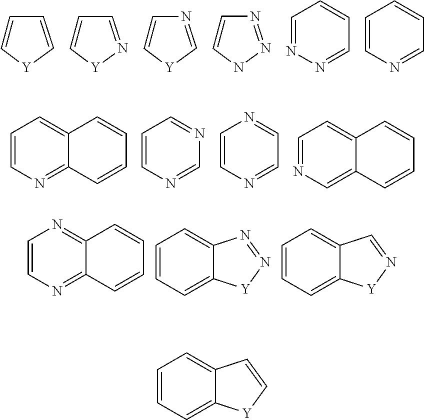

wherein each Y is selected from carbonyl, N, NR⁶⁵, O and S; and R⁶⁵ is independently hydrogen, C₁-C₈ alkyl, C₃-C₁₀ cycloalkyl, 4-10 membered heterocycloalkyl, C₆-C₁₀ aryl, and 5-10 membered heteroaryl.

Examples of representative aryl having hetero atoms containing substitution include the following:

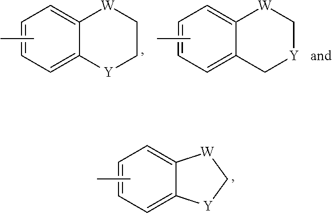

wherein each W is selected from C(R⁶⁶)₂, NR⁶⁶, O and S; and each Y is selected from carbonyl, NR⁶⁶, O and S; and R⁶⁶ is independently hydrogen, C₁-C₈ alkyl, C₃-C₁₀ cycloalkyl, 4-10 membered heterocycloalkyl, C₆-C₁₀ aryl, and 5-10 membered heteroaryl.

As used herein, the term ‘heterocycloalkyl’ refers to a 4-10 membered, stable heterocyclic non-aromatic ring and/or including rings containing one or more heteroatoms independently selected from N, O and S, fused thereto. A fused heterocyclic ring system may include carbocyclic rings and need only include one heterocyclic ring. Examples of heterocyclic rings include, but are not limited to, morpholine, piperidine (e.g. 1-piperidinyl, 2-piperidinyl, 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Further examples include thiomorpholine and its S-oxide and S,S-dioxide (particularly thiomorpholine). Still further examples include azetidine, piperidone, piperazone, and N-alkyl piperidines such as N-methyl piperidine. Particular examples of heterocycloalkyl groups are shown in the following illustrative examples:

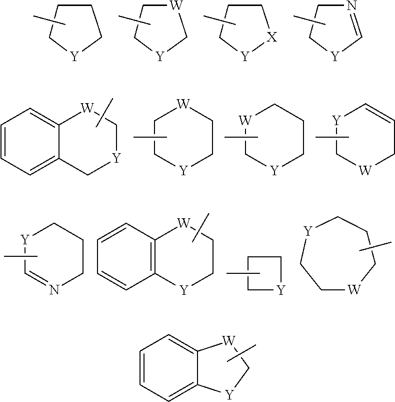

wherein each W is selected from CR⁶⁷, C(R⁶⁷)₂, NR⁶⁷, O and S; and each Y is selected from NR⁶⁷, O and S; and R⁶⁷ is independently hydrogen, C₁-C₈ alkyl, C₃-C₁₀ cycloalkyl, 4-10 membered heterocycloalkyl, C₆-C₁₀ aryl, 5-10 membered heteroaryl, These heterocycloalkyl rings may be optionally substituted with one or more groups selected from the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

‘Hydroxy’ refers to the radical —OH.

‘Nitro’ refers to the radical —NO₂.

‘Substituted’ refers to a group in which one or more hydrogen atoms are each independently replaced with the same or different substituent(s). Typical substituents may be selected from the group consisting of:

halogen, —R⁶⁸, —O⁻, ═O, —OR⁶⁸, —SR⁶⁸, —S⁻, ═S, —NR⁶⁸R⁶⁹, ═NR⁶⁸, —CCl₃, —CF₃, —CN, —OCN, —SCN, —NO, —NO₂, ═N₂, —N₃, —S(O)₂O⁻, —S(O)₂OH, —S(O)₂R⁶⁸, —OS(O₂)O⁻, —OS(O)₂R⁶⁸, —P(O)(O⁻)₂, —P(O)(OR⁶⁸)(O⁻), —OP(O)(OR⁶⁸)(OR⁶⁹), —C(O)R⁶⁸, —C(S)R⁶⁸, —C(O)OR⁶⁸, —C(O)NR⁶⁸R⁶⁹, —C(O)O⁻, —C(S)OR⁶⁸, —NR⁷⁰C(O)NR⁶⁸R⁶⁹, —NR⁷⁰C(S)NR⁶⁸R⁶⁹, —NR⁷¹C(NR⁷⁰)NR⁶⁸R⁶⁹ and —C(NR⁷⁰)NR⁶⁸R⁶⁹;
wherein each R⁶⁸, R⁶⁹, R⁷⁰ and R⁷¹ are independently:
hydrogen, C₁-C₈ alkyl, C₆-C₁₀ aryl, arylalkyl, C₃-C₁₀ cycloalkyl, 4-10 membered heterocycloalkyl, 5-10 membered heteroaryl, heteroarylalkyl; or
C₁-C₈ alkyl substituted with halo or hydroxy; or
C₆-C₁₀ aryl, 5-10 membered heteroaryl, C₆-C₁₀ cycloalkyl or 4-10 membered heterocycloalkyl each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

In a particular embodiment, substituted groups are substituted with one or more substituents, particularly with 1 to 3 substituents, in particular with one substituent group.

In a further particular embodiment the substituent group or groups are selected from halo, cyano, nitro, trifluoromethyl, trifluoromethoxy, azido, —NR⁷²SO₂R⁷³, —SO₂NR⁷³R⁷², —C(O)R⁷³, —C(O)OR⁷³, —OC(O)R⁷³, —NR⁷²C(O)R⁷³, —C(O)NR⁷³R⁷², —NR⁷³R⁷², —(CR⁷²R⁷²)ₘOR⁷², wherein, each R⁷³ is independently selected from H, C₁-C₈ alkyl, —(CH₂)ₜ(C₆-C₁₀ aryl), —(CH₂)ₜ(5-10 membered heteroaryl), —(CH₂)ₜ(C₃-C₁₀ cycloalkyl), and —(CH₂)ₜ(4-10 membered heterocycloalkyl), wherein t is an integer from 0 to 4; and
any alkyl groups present, may themselves be substituted by halo or hydroxy; and
any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Each R″ independently represents H or C₁-C₆alkyl.

‘Substituted sulfanyl’ refers to the group —SR⁷⁴, wherein R⁷⁴ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘substituted sulfanyl’ groups are —S—(C₁-C₈ alkyl) and —S—(C₃-C₁₀ cycloalkyl), —S—(CH₂)ₜ(C₆-C₁₀ aryl), —S—(CH₂)ₜ(5-10 membered heteroaryl), —S—(CH₂)ₜ(C₃-C₁₀ cycloalkyl), and —S—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. The term ‘substituted sulfanyl’ includes the groups ‘alkylsulfanyl’ or ‘alkylthio’, ‘substituted alkylthio’ or ‘substituted alkylsulfanyl’, ‘cycloalkylsulfanyl’ or ‘cycloalkylthio’, ‘substituted cycloalkylsulfanyl’ or ‘substituted cycloalkylthio’, ‘arylsulfanyl’ or ‘arylthio’ and ‘heteroarylsulfanyl’ or ‘heteroarylthio’ as defined below.

‘Alkylthio’ or ‘Alkylsulfanyl’ refers to a radical —SR⁷⁵ where R⁷⁵ is a C₁-C₈ alkyl or group as defined herein. Representative examples include, but are not limited to, methylthio, ethylthio, propylthio and butylthio.

‘Substituted Alkylthio’ or ‘substituted alkylsulfanyl’ refers to the group —SR⁷⁶ where R⁷⁶ is a C₁-C₈ alkyl, substituted with halo, substituted or unsubstituted amino, or hydroxy.

‘Cycloalkylthio’ or ‘Cycloalkylsulfanyl’ refers to a radical —SR⁷⁷ where R⁷⁷ is a C₃-C₁₀ cycloalkyl or group as defined herein. Representative examples include, but are not limited to, cyclopropylthio, cyclohexylthio, and cyclopentylthio.

‘Substituted cycloalkylthio’ or ‘substituted cycloalkylsulfanyl’ refers to the group —SR⁷⁸ where R⁷⁸ is a C₃-C₁₀ cycloalkyl, substituted with halo, substituted or unsubstituted amino, or hydroxy.

‘Arylthio’ or ‘Arylsulfanyl’ refers to a radical —SR⁷⁹ where R⁷⁹ is a C₆-C₁₀ aryl group as defined herein.

‘Heteroarylthio’ or ‘Heteroarylsulfanyl’ refers to a radical —SR⁸⁰ where R⁸⁰ is a 5-10 membered heteroaryl group as defined herein.

‘Substituted sulfinyl’ refers to the group —S(O)R⁸¹, wherein R⁸¹ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘substituted sulfinyl’ groups are —S(O)—(C₁-C₈ alkyl) and —S(O)—(C₃-C₁₀ cycloalkyl), —S(O)—(CH₂)ₜ(C₆-C₁₀ aryl), —S(O)—(CH₂)ₜ(5-10 membered heteroaryl), —S(O)—(CH₂)ₜ(C₃-C₁₀ cycloalkyl), and —S(O)—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. The term substituted sulfinyl includes the groups ‘alkylsulfinyl’, ‘substituted alkylsulfinyl’, ‘cycloalkylsulfinyl’, ‘substituted cycloalkylsulfinyl’, ‘arylsulfinyl’ and ‘heteroarylsulfinyl’ as defined herein.

‘Alkylsulfinyl’ refers to a radical —S(O)R⁸² where R⁸² is a C₁-C₈ alkyl group as defined herein. Representative examples include, but are not limited to, methylsulfinyl, ethylsulfinyl, propylsulfinyl and butylsulfinyl.

‘Substituted Alkylsulfinyl’ refers to a radical —S(O)R⁸³ where R⁸³ is a C₁-C₈ alkyl group as defined herein, substituted with halo, substituted or unsubstituted amino, or hydroxy.

‘Cycloalkylsulfinyl’ refers to a radical —S(O)R⁸⁴ where R⁸⁴ is a C₃-C₁₀ cycloalkyl or group as defined herein. Representative examples include, but are not limited to, cyclopropylsulfinyl, cyclohexylsulfinyl, and cyclopentylsulfinyl. Exemplary ‘cycloalkylsulfinyl’ groups are S(O)—C₃-C₁₀ cycloalkyl.

‘Substituted cycloalkylsulfinyl’ refers to the group —S(O)R⁸⁵ where R⁸⁵ is a C₃-C₁₀ cycloalkyl, substituted with halo, substituted or unsubstituted amino, or hydroxy.

‘Arylsulfinyl’ refers to a radical —S(O)R⁸⁶ where R⁸⁶ is a C₆-C₁₀ aryl group as defined herein.

‘Heteroarylsulfinyl’ refers to a radical —S(O)R⁸⁷ where R⁸⁷ is a 5-10 membered heteroaryl group as defined herein.

‘Substituted sulfonyl’ refers to the group —S(O)₂R⁸⁸, wherein R⁸⁸ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘substituted sulfonyl’ groups are —S(O)₂—(C₁-C₈ alkyl) and —S(O)₂—(C₃-C₁₀ cycloalkyl), —S(O)₂—(CH₂)ₜ(C₆-C₁₀ aryl), —S(O)₂—(CH₂)ₜ(5-10 membered heteroaryl), —S(O)₂—(CH₂)ₜ(C₃-C₁₀ cycloalkyl), and —S(O)₂—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. The term substituted sulfonyl includes the groups alkylsulfonyl, substituted alkylsulfonyl, cycloalkylsulfonyl, substituted cycloalkylsulfonyl, arylsulfonyl and heteroarylsulfonyl.

‘Alkylsulfonyl’ refers to a radical —S(O)₂R⁸⁹ where R⁸⁹ is an C₁-C₈ alkyl group as defined herein. Representative examples include, but are not limited to, methylsulfonyl, ethylsulfonyl, propylsulfonyl and butylsulfonyl.

‘Substituted Alkylsulfonyl’ refers to a radical —S(O)₂R⁹⁰ where R⁹⁰ is an C₁-C₈ alkyl group as defined herein, substituted with halo, substituted or unsubstituted amino, or hydroxy.

‘Cycloalkylsulfonyl’ refers to a radical —S(O)₂R⁹¹ where R⁹¹ is a C₃-C₁₀ cycloalkyl or group as defined herein. Representative examples include, but are not limited to, cyclopropylsulfonyl, cyclohexylsulfonyl, and cyclopentylsulfonyl.

‘Substituted cycloalkylsulfonyl’ refers to the group —S(O)₂R⁹² where R⁹² is a C₃-C₁₀ cycloalkyl, substituted with halo, substituted or unsubstituted amino, or hydroxy.

‘Arylsulfonyl’ refers to a radical —S(O)₂R⁹³ where R⁹³ is an C₆-C₁₀ aryl group as defined herein.

‘Heteroarylsulfonyl’ refers to a radical —S(O)₂R⁹⁴ where R⁹⁴ is an 5-10 membered heteroaryl group as defined herein.

‘Sulfo’ or ‘sulfonic acid’ refers to a radical such as —SO₃H.

‘Substituted sulfo’ or ‘sulfonic acid ester’ refers to the group —S(O)₂OR⁹⁵, wherein R⁹⁵ is selected from:
C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; or
C₁-C₈ alkyl substituted with halo, substituted or unsubstituted amino, or hydroxy; or
C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

Exemplary ‘Substituted sulfo’ or ‘sulfonic acid ester’ groups are —S(O)₂—O—(C₁-C₈ alkyl) and —S(O)₂—O—(C₃-C₁₀ cycloalkyl), —S(O)₂—O—(CH₂)ₜ(C₆-C₁₀ aryl), —S(O)₂—O—(CH₂)ₜ(5-10 membered heteroaryl), —S(O)₂—O—(CH₂)ₜ(C₃-C₁₀ cycloalkyl), and —S(O)₂—O—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

‘Thiol’ refers to the group —SH.

‘Aminocarbonylamino’ refers to the group —NR⁹⁶C(O)NR⁹⁶R⁹⁶ where each R⁹⁶ is independently hydrogen C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl, as defined herein; or where two R⁹⁶ groups, when attached to the same N, are joined to form an alkylene group.

‘Bicycloaryl’ refers to a monovalent aromatic hydrocarbon group derived by the removal of one hydrogen atom from a single carbon atom of a parent bicycloaromatic ring system. Typical bicycloaryl groups include, but are not limited to, groups derived from indane, indene, naphthalene, tetrahydronaphthalene, and the like. Particularly, an aryl group comprises from 8 to 11 carbon atoms.

‘Bicycloheteroaryl’ refers to a monovalent bicycloheteroaromatic group derived by the removal of one hydrogen atom from a single atom of a parent bicycloheteroaromatic ring system. Typical bicycloheteroaryl groups include, but are not limited to, groups derived from benzofuran, benzimidazole, benzindazole, benzdioxane, chromene, chromane, cinnoline, phthalazine, indole, indoline, indolizine, isobenzofuran, isochromene, isoindole, isoindoline, isoquinoline, benzothiazole, benzoxazole, naphthyridine, benzoxadiazole, pteridine, purine, benzopyran, benzopyrazine, pyridopyrimidine, quinazoline, quinoline, quinolizine, quinoxaline, benzomorphan, tetrahydroisoquinoline, tetrahydroquinoline, and the like. Preferably, the bicycloheteroaryl group is between 9-11 membered bicycloheteroaryl, with 5-10 membered heteroaryl being particularly preferred. Particular bicycloheteroaryl groups are those derived from benzothiophene, benzofuran, benzothiazole, indole, quinoline, isoquinoline, benzimidazole, benzoxazole and benzdioxane.

‘Compounds of the present invention’, and equivalent expressions, are meant to embrace the compounds as hereinbefore described, in particular compounds according to any of the formulae herein recited and/or described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

‘Cycloalkylalkyl’ refers to a radical in which a cycloalkyl group is substituted for a hydrogen atom of an alkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

‘Heterocycloalkylalkyl’ refers to a radical in which a heterocycloalkyl group is substituted for a hydrogen atom of an alkyl group. Typical heterocycloalkylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

‘Cycloalkenyl’ refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

‘Substituted cycloalkenyl’ refers to those groups recited in the definition of “substituted” herein, and particularly refers to a cycloalkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Fused Cycloalkenyl’ refers to a cycloalkenyl having two of its ring carbon atoms in common with a second aliphatic or aromatic ring and having its olefinic unsaturation located to impart aromaticity to the cycloalkenyl ring.

‘Ethenyl’ refers to substituted or unsubstituted —(C═C)—.

‘Ethylene’ refers to substituted or unsubstituted —(C—C)—.

‘Ethynyl’ refers to —(C≡C)—.

‘Hydrogen bond donor’ group refers to a group containing O—H, or N—H functionality. Examples of ‘hydrogen bond donor’ groups include —OH, —NH₂, and —NH—R⁹⁷ and wherein R⁹⁷ is alkyl, acyl, cycloalkyl, aryl, or heteroaryl.

‘Dihydroxyphosphoryl’ refers to the radical —PO(OH)₂.

‘Substituted dihydroxyphosphoryl’ refers to those groups recited in the definition of “substituted” herein, and particularly refers to a dihydroxyphosphoryl radical wherein one or both of the hydroxyl groups are substituted. Suitable substituents are described in detail below.

‘Aminohydroxyphosphoryl’ refers to the radical —PO(OH)NH₂.

‘Substituted aminohydroxyphosphoryl’ refers to those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

‘Nitrogen-Containing Heterocycloalkyl’ group means a 4 to 7 membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

‘Thioketo’ refers to the group ═S.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Therapeutically effective amount’ means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

‘Preventing’ or ‘prevention’ refers to a reduction in risk of acquiring or developing a disease or disorder (i.e., causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

‘Compounds of the present invention’, and equivalent expressions, are meant to embrace compounds of the Formula (e) as hereinbefore described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

When ranges are referred to herein, for example but without limitation, C₁-C₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₅ alkyl, C₂-C₈ alkenyl, aryl, C₇-C₁₂ substituted aryl, and C₇-C₁₂ arylalkyl esters of the compounds of the invention.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compounds

In certain aspects, the present invention provides compounds useful for preventing and/or treating a broad range of conditions, among them, pain, sleep disorders, anxiety and depression disorders, weight and eating disorders, Parkinson's disease, addiction, spasticity, inflammatory disorders, glaucoma or other disorders.

Accordingly, in one aspect, compounds are provided that have formula I:

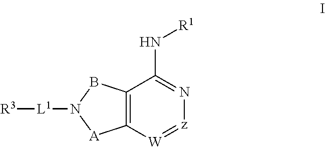

wherein
each A and B is independently CR^{2a}R^{2b};
W and Z are independently N or CR⁴;
L¹ is a single bond or substituted or unsubstituted C₁-C₅ alkylene, —CO—, —NHC(O)—, —OC(O)—, —SO—, or S(O)₂—;
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof; and stereoisomers, isotopic variants and tautomers thereof;
provided that the compound is other than
N-[5-(1,1-dimethylethyl)-2-methylphenyl]-6,7-dihydro-2-[4-(4-methoxyphenyl)-1-piperazinyl]-6-(methylsulfonyl)-5H-pyrrolo[3,4-d]pyrimidin-4-amine;
4-[[5-(1,1-dimethylethyl)-2-methylphenyl]amino]-5,7-dihydro-2-[4-(4-methoxyphenyl)-1-piperazinyl]-6H-Pyrrolo[3,4-d]pyrimidine-6-carboxylic acid, 1,1-dimethylethyl ester;
2-chloro-4-[[3-(dimethylamino)-5-methoxyphenyl]amino]-5,7-dihydro-6H-pyrrolo[3,4-d]pyrimidine-6-carboxylic acid, 9H-fluoren-9-ylmethyl ester;
2-chloro-5,7-dihydro-4-[(5-methyl-1H-pyrazol-3-yl)amino]-6H-pyrrolo[3,4-d]pyrimidine-6-carboxylic acid, 9H-fluoren-9-ylmethyl ester; and
N-(2,3-dihydro-1H-inden-2-yl)-6,7-dihydro-2-(methylthio)-6-(phenylmethyl)-5H-pyrrolo[3,4-d]pyrimidin-4-amine.

In another aspect, compounds are provided that have formula I:

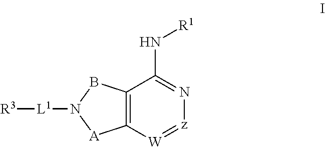

wherein
each A and B is independently CR^{2a}R^{2b};
W and Z are independently N or CR⁴;
L¹ is —CO—, —NHC(O)—, —OC(O)—, —SO—, or S(O)₂—;
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof; and stereoisomers, isotopic variants and tautomers thereof;
provided that the compound is other than
N-[5-(1,1-dimethylethyl)-2-methylphenyl]-6,7-dihydro-2-[4-(4-methoxyphenyl)-1-piperazinyl]-6-(methylsulfonyl)-5H-pyrrolo[3,4-d]pyrimidin-4-amine;
4-[[5-(1,1-dimethylethyl)-2-methylphenyl]amino]-5,7-dihydro-2-[4-(4-methoxyphenyl)-1-piperazinyl]-6H-Pyrrolo[3,4-d]pyrimidine-6-carboxylic acid, 1,1-dimethylethyl ester;
2-chloro-4-[[3-(dimethylamino)-5-methoxyphenyl]amino]-5,7-dihydro-6H-pyrrolo[3,4-d]pyrimidine-6-carboxylic acid, 9H-fluoren-9-ylmethyl ester; and
2-chloro-5,7-dihydro-4-[(5-methyl-1H-pyrazol-3-yl)amino]-6H-pyrrolo[3,4-d]pyrimidine-6-carboxylic acid, 9H-fluoren-9-ylmethyl ester.

In yet another aspect, compounds are provided that have formula I:

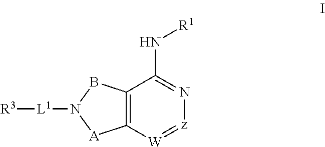

wherein
each A and B is independently CR^{2a}R^{2b};
W and Z are independently N or CR⁴;
L¹ is a single bond or substituted or unsubstituted C₁-C₅ alkylene
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof;
and stereoisomers, isotopic variants and tautomers thereof;
provided that the compound is other than N-(2,3-dihydro-1H-inden-2-yl)-6,7-dihydro-2-(methylthio)-6-(phenylmethyl)-5H-pyrrolo[3,4-d]pyrimidin-4-amine.

In one particular embodiment, with respect to compounds of formula I, A is CR^{2a}R^{2b}.

In one particular embodiment, with respect to compounds of formula I, A is CHR^{2b}.

In one particular embodiment, with respect to compounds of formula I, A is CH₂.

In one particular embodiment, with respect to compounds of formula I, B is CR^{2a}R^{2b}. In another embodiment, B is CHR^{2b}.

In one particular embodiment, with respect to compounds of formula I, B is CH₂.

In one particular embodiment, with respect to compounds of formula I, B is CH₂.

In one particular embodiment, with respect to compounds of formula I, R¹ is substituted or unsubstituted aryl.

In one particular embodiment, with respect to compounds of formula I, R¹ is substituted or unsubstituted phenyl.

In one particular embodiment, with respect to compounds of formula I, R¹ is substituted or unsubstituted heteroaryl.

In one particular embodiment, with respect to compounds of formula I, R¹ is substituted or unsubstituted pyridyl.

In one particular embodiment, with respect to compounds of formula I, R¹ is unsubstituted phenyl. In another embodiment, R¹ is unsubstituted pyridyl.

In one particular embodiment, with respect to compounds of formula I, R¹ is phenyl, pyridyl or quinolin-3-yl, substituted with one or more substituents independently selected from halo, C₁-C₆alkyl, haloC₁-C₆alkyl, C₃-C₈cycloalkyl, amino, aryl, heteroaryl, cyano, hydroxy, alkoxy and substituted sulfonyl.

In one particular embodiment, with respect to compounds of formula I, R¹ is phenyl, pyridyl or quinolin-3-yl, substituted with one or more substituents independently selected from Me, Et, Ph, Cl, F, Br, CN, OH, OMe, OEt, OPh, COPh, CF₃, CHF₂, OCF₃, i-Pr, i-Bu, t-Bu, SMe, CH═CH—CO₂H, SOMe, SO₂Me, SO₃H, SO₃Me, and pyridyl.

In yet another aspect, compounds are provided that have formula I:

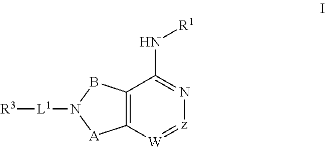

wherein
each A and B is independently CR^{2a}R^{2b};
W and Z are independently N or CR⁴;
L¹ is a single bond or substituted or unsubstituted C₁-C₅ alkylene, —CO—, —NHC(O)—, —OC(O)—, —SO—, or S(O)₂—;
R¹ is selected from a substituted or unsubstituted bicycloaryl or bicycloheteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof; and stereoisomers, isotopic variants and tautomers thereof;

In one particular embodiment, with respect to compounds of formula I, R¹ is substituted or unsubstituted naphthylene.

In one particular embodiment, with respect to compounds of formula I, R¹ is substituted or unsubstituted bicycloheteroaryl.

In one particular embodiment, with respect to compounds of formula I, R¹ is substituted or unsubstituted quinoline.

In one particular embodiment, with respect to compounds of formula I, R¹ is substituted or unsubstituted isoquinoline.

In one particular embodiment, with respect to compounds of formula I, R¹ is selected from substituted or unsubstituted benzodioxole, substituted or unsubstituted benzodioxane, substituted or unsubstituted benzofuran, substituted or unsubstituted benzothiophene, and substituted or unsubstituted benzodioxepine.

In one particular embodiment, with respect to compounds of formula I, R¹ is substituted or unsubstituted quinolin-3-yl.

In one particular embodiment, with respect to compounds of formula I, R¹ is unsubstituted quinolin-3-yl.

In one particular embodiment, with respect to compounds of formula I, L¹ is a single bond.

In one particular embodiment, with respect to compounds of formula I, L¹ is a C₁-C₅ alkylene group.

In one particular embodiment, with respect to compounds of formula I, L¹ is —CH₂—, —C(Me)H—, —CH₂CH₂—, —C(Me)HCH₂—, or —CH₂C(Me)H—.

In one particular embodiment, with respect to compounds of formula I, L¹ is —CO—, —NHC(O)—, or —OC(O)—.

In one particular embodiment, with respect to compounds of formula I, L¹ is —SO—.

In one particular embodiment, with respect to compounds of formula I, L¹ is —S(O)₂—.

In one particular embodiment, with respect to compounds of formula I, W is CR⁴ and R⁴ is H, substituted or unsubstituted C₁-C₆alkyl, or halo.

In one particular embodiment, with respect to compounds of formula I, W is CR⁴ and R⁴ is H, Me, CF₃, Cl or F.

In one particular embodiment, with respect to compounds of formula I, W is N.

In one particular embodiment, with respect to compounds of formula I, Z is CR⁴ and R⁴ is H, substituted or unsubstituted C₁-C₆alkyl, or halo.

In one particular embodiment, with respect to compounds of formula I, Z is CR⁴ and R⁴ is H, Me, CF₃, Cl or F.

In one particular embodiment, with respect to compounds of formula I, each of W and Z is independently CH.

In one particular embodiment, with respect to compounds of formula I, W is N, and Z is CH.

In one particular embodiment, with respect to compounds of formula I, the compound is according to formula IIa, IIb, IIc, IId, IIe, IIf, IIg, IIh, Iii, or IIj:

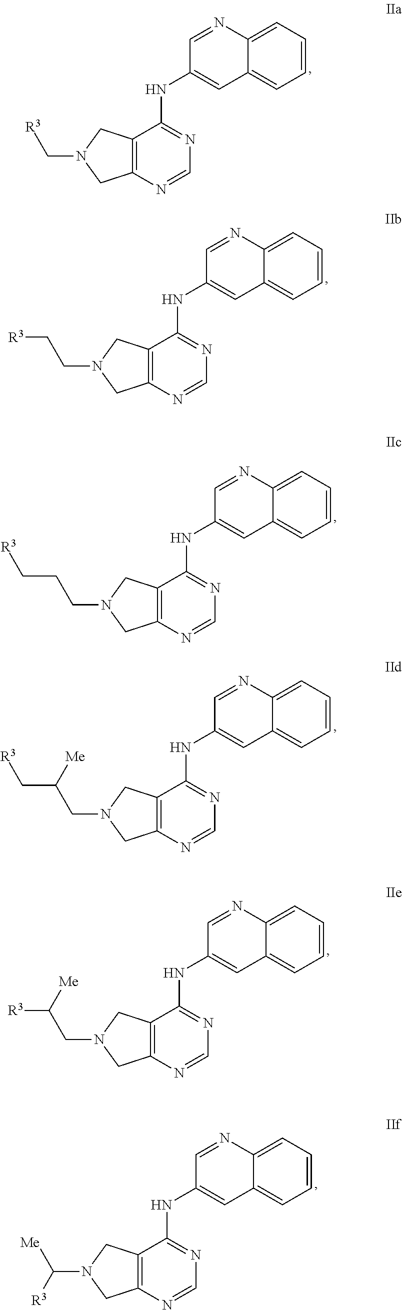

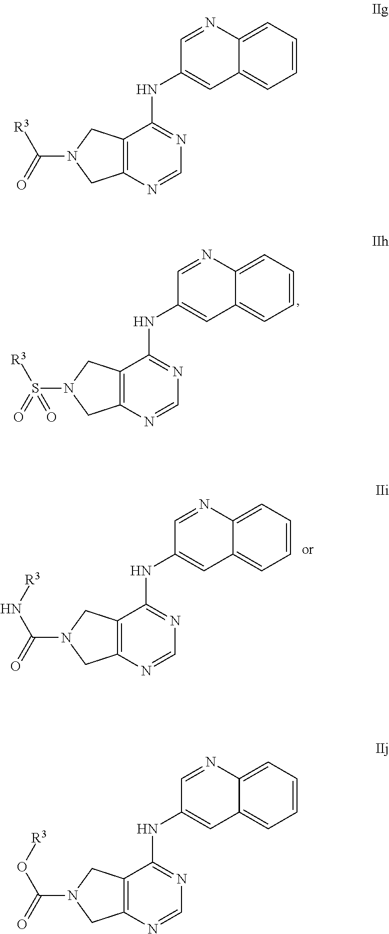

and wherein R³ is as described for formula I.

In one particular embodiment, with respect to compounds of formula I, the compound is according to formula IIa, IIb, IIc, IId, IIe or IIf:

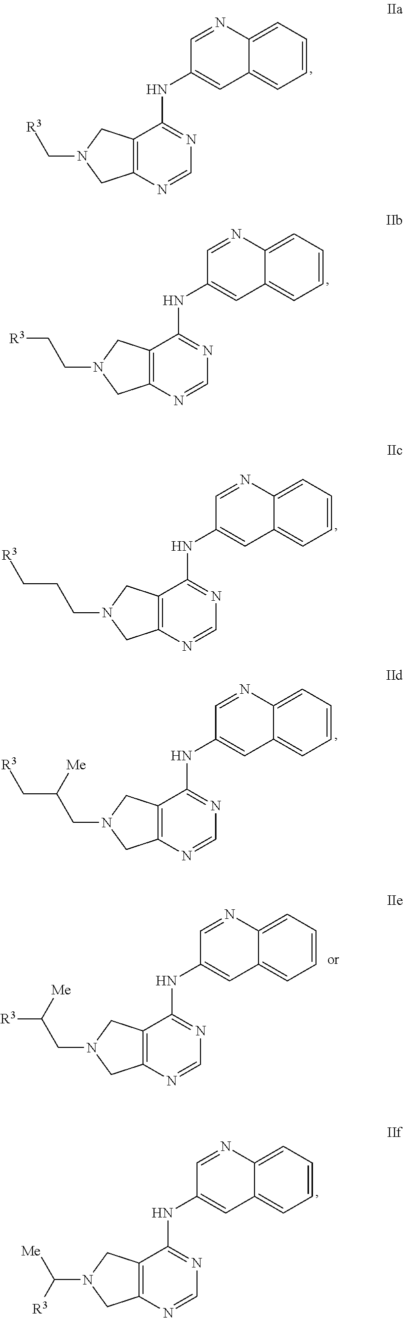

and wherein R³ is as described for formula I.

In one particular embodiment, with respect to compounds of formula I, the compound is according to formula IIa, IIb, IIc, IId, IIe, or IIf.

In one particular embodiment, with respect to compounds of formula I-IIf, R³ is substituted or unsubstituted C₁-C₆alkyl.

In one particular embodiment, with respect to compounds of formula I-IIf, R³ is unsubstituted C₁-C₆alkyl.

In one particular embodiment, with respect to compounds of formula I-IIf, R³ is Me, Et, n-Pr, i-Pr, n-Bu, t-Bu, 1-methylpropyl, 2-methylpropyl, 2,2-dimethylpropyl, or CF₃.

In one particular embodiment, with respect to compounds of formula I-IIf, R³ is C₁-C₆alkyl, substituted with halo, hydroxy or alkoxy. In another embodiment, R³ is C₁-C₆alkyl, substituted with Cl, F or OH.

In one particular embodiment, with respect to compounds of formula I-IIf, R³ is CH₂OH.

In one particular embodiment, with respect to compounds of formula I-IIf, R³ is CH₂Ph. In another particular embodiment, R³ is CH₂-(2-Cl-Ph). In yet another particular embodiment, R³ is CH₂-(2,4-diF-Ph).

In one particular embodiment, with respect to compounds of formula I-IIf, R³ is C₃-C₈cycloalkyl, unsubstituted or substituted with one or more substituents independently selected from halo, hydroxyl, C₁-C₆alkyl, alkoxy and haloalkyl.

In one particular embodiment, with respect to compounds of formula I-IIf, R³ is unsubstituted cyclopropyl, cyclobutyl, cyclopentyl or cyclohexyl.

In one particular embodiment, with respect to compounds of formula I-IIf, R³ is unsubstituted cyclobutyl.

In one particular embodiment, with respect to compounds of formula I-IIf, R³ is unsubstituted cyclopentyl.

In one particular embodiment, with respect to compounds of formula I-IIf, R³ is unsubstituted cyclohexyl.

In one particular embodiment, with respect to compounds of formula I-IIf, R³ is unsubstituted cyclobutyl.

In one particular embodiment, with respect to compounds of formula I-IIf, R³ is unsubstituted cyclohexyl.

In one particular embodiment, with respect to compounds of formula I-IIf, R³ is cyclopropyl, cyclobutyl, cyclopentyl or cyclohexyl, substituted with one or more substituents independently selected from halo, hydroxyl, C₁-C₆alkyl, alkoxy and haloalkyl.

In one particular embodiment, with respect to compounds of formula I-IIf, R³ is cyclopropyl, cyclobutyl, cyclopentyl or cyclohexyl, substituted with one or more substituents independently selected from Me, Et, Cl, F, CN, OH, OMe, OEt, CF₃, CHF₂, OCF₃, i-Pr, i-Bu, and t-Bu.

In one particular embodiment, with respect to compounds of formula I-IIf, R³ is substituted or unsubstituted heterocycloalkyl.

In one particular embodiment, with respect to compounds of formula I-IIf, R³ is unsubstituted pyrrolidinyl, piperidinyl, morpholinyl, tetrahydropyranyl, or piperazinyl.

In one particular embodiment, with respect to compounds of formula I-IIf, R³ is pyrrolidinyl, piperidinyl, morpholinyl, tetrahydropyranyl, or piperazinyl, substituted with one or more substituents independently selected from halo, hydroxyl, C₁-C₆alkyl, alkoxy and haloC₁-C₆alkyl.

In one particular embodiment, with respect to compounds of formula I-IIf, R³ is pyrrolidinyl, piperidinyl, morpholinyl, or piperazinyl, substituted with one or more substituents independently selected from Me, Et, Cl, F, CN, OH, OMe, OEt, CF₃, CHF₂, OCF₃, i-Pr, i-Bu, and t-Bu.

In one particular embodiment, with respect to compounds of formula I-IIf, R³ is pyrrolidinyl, piperidinyl, morpholinyl, or piperazinyl, substituted with Me.

In one particular embodiment, with respect to compounds of formula I-IIf, R³ is pyrrolidinyl, piperidinyl, morpholinyl, or piperazinyl, unsubstituted or substituted with Me.

In one particular embodiment, with respect to compounds of formula I-IIf, R³ is substituted or unsubstituted aryl or heteroaryl.

In one particular embodiment, with respect to compounds of formula I-IIf, R³ is phenyl, unsubstituted or substituted with one or more substituents independently selected from halo, hydroxyl, amino, cyano, sulfo, substituted sulfanyl, substituted sulfinyl, substituted sulfonyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

In one particular embodiment, with respect to compounds of formula I-IIf, R³ is phenyl substituted with one or more substituents independently selected from Me, Et, Ph, Cl, F, Br, CN, OH, OMe, OEt, OPh, COPh, CF₃, CHF₂, OCF₃, i-Pr, i-Bu, t-Bu, SMe, CH═CH—CO₂H, SOMe, SO₂Me, SO₃H, SO₃Me, and pyridyl. In one particular embodiment, R³ is monosubstituted phenyl. In another embodiment R³ is disubstituted phenyl.

In one particular embodiment, with respect to compounds of formula I-IIf, R³ is Ph, 2-Cl-Ph, 2-F-Ph, 4-Cl-Ph, 4-F-Ph, 2,4-dichlorophenyl, 2,4-difluorophenyl, 4-OH-Ph, or 2-OH-Ph.

In one particular embodiment, with respect to compounds of formula I-IIf, R³ is Ph substituted with substituted or unsubstituted amino. In another particular embodiment, R³ is Ph substituted with NH₂.

In one particular embodiment, with respect to compounds of formula I-IIf, R³ is Ph, 2-Cl-Ph, 2-F-Ph, 4-Cl-Ph, 4-F-Ph, 2,4-dichlorophenyl, 2,4-difluorophenyl, 4-OH-Ph, or 2-OH-Ph. In another particular embodiment, R³ is 4-OH-Ph.

In one particular embodiment, with respect to compounds of formula I-IIf, R³ is Ph substituted with Cl. In another particular embodiment, R³ is 2-Cl-Ph, 3-Cl-Ph, or 4-Cl-Ph. In yet another particular embodiment, R³ is 2-Cl-Ph.

In one particular embodiment, with respect to compounds of formula I-IIf, R³ is Ph substituted with F. In another particular embodiment, R³ is 2-F-Ph, 3-F-Ph, or 4-F-Ph. In yet another particular embodiment, R³ is 2-F-Ph.

In one particular embodiment, with respect to compounds of formula I-IIf, R³ is Ph substituted with OCF₃. In another particular embodiment, R³ is 3-OCF₃-Ph. In yet another particular embodiment, R³ is 2-OCF₃-Ph.

In one particular embodiment, with respect to compounds of formula I-IIf, R³ is Ph substituted with CF₃. In another particular embodiment, R³ is 3-CF₃-Ph. In yet another particular embodiment, R³ is 2-CF₃-Ph or 4-CF₃-Ph.

In one particular embodiment, with respect to compounds of formula I-IIf, R³ is 2,4-difluorophenyl.

In one particular embodiment, with respect to compounds of formula I-IIf, R³ is Ph substituted with NO₂. In another particular embodiment, R³ is 2-NO₂-Ph, 3-NO₂-Ph, or 4-NO₂-Ph. In yet another particular embodiment, R³ is 2-NO₂Ph. In yet another particular embodiment, R³ is 2,4-di-NO₂Ph.

In one particular embodiment, with respect to compounds of formula I-IIf, R³ is pyridyl, unsubstituted or substituted with one or more substituents independently selected from halo, hydroxyl, amino, cyano, sulfo, substituted sulfanyl, substituted sulfinyl, substituted sulfonyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

In one particular embodiment, with respect to compounds of formula I-IIf, R³ is pyridyl substituted with one or more substituents independently selected from Me, Et, Ph, Cl, F, Br, CN, OH, OMe, OEt, OPh, COPh, CF₃, CHF₂, OCF₃, i-Pr, i-Bu, t-Bu, SMe, CH═CH—CO₂H, SOMe, SO₂Me, SO₃H, SO₃Me, and pyridyl.

In one particular embodiment, with respect to compounds of formula I-IIf, R³ is unsubstituted pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl.

In one particular embodiment, with respect to compounds of formula I-IIf, R³ is unsubstituted indolyl, indazolyl, thiadiazolyl, or furanyl.

In one particular embodiment, with respect to compounds of formula I-IIf, R³ is pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl, substituted with one or more substituents independently selected from halo, hydroxyl, amino, cyano, sulfo, substituted sulfonyl, substituted sulfanyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

In one particular embodiment, with respect to compounds of formula I-IIf, R³ is pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl, substituted with one or more substituents independently selected from Me, Et, Ph, Cl, F, CN, OH, OMe, OEt, OPh, COPh, CF₃, CHF₂, OCF₃, i-Pr, i-Bu, t-Bu, SMe, CO₂Me, CO₂Et, CH═CH—CO₂H, SOMe, SO₂Me, SO₃H, SO₃Me, and pyridyl.

In one particular embodiment, with respect to compounds of formula I-IIf, R³ is unsubstituted thienyl, furanyl, pyrrolyl, pyrazolyl, imidazolyl, thiazolyl, thiadiazolyl, or oxadiazolyl.

In one particular embodiment, with respect to compounds of formula I-IIf, R³ is thienyl, furanyl, pyrrolyl, pyrazolyl, imidazolyl, thiazolyl, thiadiazolyl, or oxadiazolyl, substituted with one or more substituents independently selected from Me, Et, Cl, CF₃, CO₂Me, CO₂Et, and NHAc.

In one particular embodiment, with respect to compounds of formula I-IIf, R³ is Ph, thienyl, furanyl, pyrrolyl, pyrazolyl, imidazolyl, thiazolyl, thiadiazolyl, or oxadiazolyl, substituted with 5-membered heteroaryl. In one particular embodiment, the 5-membered heteroaryl is selected from pyrrolyl, thiophenyl, oxazolyl, pyrazolyl, thiazolyl, and thiadiazolyl. In another particular embodiment, the 5-membered heteroaryl substituted with halo or C₁-C₆ alkyl. In yet another particular embodiment, the 5-membered heteroaryl is selected from pyrrolyl, thiophenyl, oxazolyl, pyrazolyl, thiazolyl, and thiadiazolyl, substituted with one or more substituents independently selected from Me, Et, Cl, and CF₃.

In one particular embodiment, with respect to compounds of formula I-IIf, R³ is thiophenyl, methylthiophenyl, furanyl, methylfuranyl, pyrazolyl, or methylpyrazolyl.

In one particular embodiment, with respect to compounds of formula I-IIf, R³ is thiadiazolyl substituted with Me, Et, Cl, or CF₃. In another particular embodiment, R³ is thiadiazolyl substituted with Cl. In one particular embodiment, R³ is 1,2,4-thiadiazolyl.

In one particular embodiment, with respect to compounds of formula I-IIf, R³ is oxadiazolyl substituted with Me, Et, Cl, CO₂Et, or CF₃. In another particular embodiment, R³ is oxadiazolyl substituted with CO₂Et. In one particular embodiment, R³ is 1,2,4-oxadiazolyl.

In one particular embodiment, with respect to compounds of formula I-IIf, R³ is furanyl, unsubstituted or substituted with Me, Et, Cl, or CF₃. In another particular embodiment, R³ is unsubstituted furanyl. In yet another particular embodiment, R³ is furanyl substituted with Cl.

In one particular embodiment, with respect to compounds of formula I-IIf, R³ is thiophenyl, unsubstituted or substituted with Me, Et, Cl, CO₂Et, or CF₃. In another particular embodiment, R³ is unsubstituted thiophenyl. In yet another particular embodiment, R³ is thiophenyl substituted with C₁ or CO₂Et.

In one particular embodiment, with respect to compounds of formula I-IIf, R³ is oxazoyl or thiazolyl, unsubstituted or substituted with Me, Et, Cl, or CF₃. In another particular embodiment, R³ is unsubstituted thiazolyl. In yet another particular embodiment, R³ is thiazoyl substituted with Me or Cl. In yet another particular embodiment, R³ is thiazoyl or oxazolyl substituted with dimethyl.

In one particular embodiment, with respect to compounds of formula I-IIf, R³ is pyrazolyl substituted with Me, Et, Cl, CO₂Et, or CF₃. In another particular embodiment, R³ is pyrazolyl substituted with CO₂Et or Me.

In one particular embodiment, with respect to compounds of formula I-IIf, R³ is thiophenyl, methylthiophenyl, furanyl, methylfuranyl, pyrazolyl, or methylpyrazolyl, substituted with one or more substituents independently selected from Me, Et, Cl, CF₃, CO₂Me, CO₂Et, and NHAc.

In one particular embodiment, with respect to compounds of formula I-IIf, R³ is thiazolyl, pyridyl, cyclohexyl, phenyl, cyclopentyl, tetrahydrothiopyranyl, pyrazolyl, tetrahydropyranyl, unsubstituted or substituted with one or two Me.

In one particular embodiment, with respect to compounds of formula I-IIf, R³ is unsubstituted thiazolyl, pyridyl, cyclohexyl, phenyl, cyclopentyl, tetrahydrothiopyranyl, pyrazolyl, tetrahydropyranyl.

In one particular embodiment, with respect to compounds of formula I-IIf, R³ is thiazolyl, pyridyl, phenyl, or pyrazolyl, substituted with Me.

In one particular embodiment, with respect to compounds of formula I-IIf, R³ is thiazolyl, or pyrazolyl, substituted with diMe.

In one particular embodiment, with respect to compounds of formula I-IIf, R³ is dimethylthiazolyl, or dimethylpyrazolyl.

In one particular embodiment, with respect to compounds of formula I, the compound is according to formula IIg or IIh:

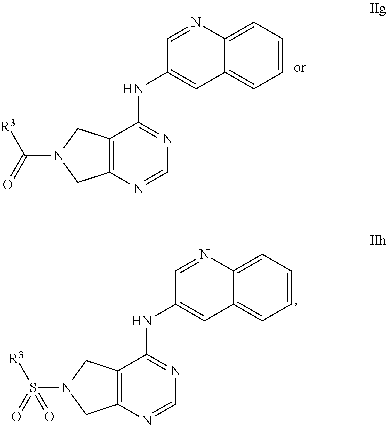

and wherein R³ is as described for formula I.

In one particular embodiment, with respect to compounds of formula I, the compound is according to formula IIg.

In one particular embodiment, with respect to compounds of formula I, the compound is according to formula IIh.

In one particular embodiment, with respect to compounds of formula I, the compound is according to formula IIi.

In one particular embodiment, with respect to compounds of formula I, the compound is according to formula IIj.

In one particular embodiment, with respect to compounds of formula IIg-IIj, R³ is substituted or unsubstituted C₁-C₆alkyl.

In one particular embodiment, with respect to compounds of formula IIg-IIj, R³ is unsubstituted C₁-C₆alkyl.

In one particular embodiment, with respect to compounds of formula IIg-IIj, R³ is Me, Et, n-Pr, i-Pr, n-Bu, t-Bu, 1-methylpropyl, 2-methylpropyl, 2,2-dimethylpropyl, or CF₃.

In one particular embodiment, with respect to compounds of formula IIg-IIj, R³ is C₁-C₆alkyl, substituted with halo, hydroxy or alkoxy. In another embodiment, R³ is C₁-C₆alkyl, substituted with Cl, F or OH.

In one particular embodiment, with respect to compounds of formula IIg-IIj, R³ is CH₂OH.

In one particular embodiment, with respect to compounds of formula IIg-IIj, R³ is CH₂Ph. In another particular embodiment, R³ is CH₂-(2-Cl-Ph). In yet another particular embodiment, R³ is CH₂-(2,4-diF-Ph).

In one particular embodiment, with respect to compounds of formula IIg-IIj, R³ is C₃-C₈cycloalkyl, unsubstituted or substituted with one or more substituents independently selected from halo, hydroxyl, C₁-C₆alkyl, alkoxy and haloalkyl.

In one particular embodiment, with respect to compounds of formula IIg-IIj, R³ is unsubstituted cyclopropyl, cyclobutyl, cyclopentyl or cyclohexyl.

In one particular embodiment, with respect to compounds of formula IIg-IIj, R³ is unsubstituted cyclobutyl.

In one particular embodiment, with respect to compounds of formula IIg-IIj, R³ is unsubstituted cyclopentyl.

In one particular embodiment, with respect to compounds of formula IIg-IIj, R³ is unsubstituted cyclohexyl.

In one particular embodiment, with respect to compounds of formula IIg-IIj, R³ is unsubstituted cyclobutyl.

In one particular embodiment, with respect to compounds of formula IIg-IIj, R³ is unsubstituted cyclohexyl.

In one particular embodiment, with respect to compounds of formula IIg-IIj, R³ is cyclopropyl, cyclobutyl, cyclopentyl or cyclohexyl, substituted with one or more substituents independently selected from halo, hydroxyl, C₁-C₆alkyl, alkoxy and haloalkyl.

In one particular embodiment, with respect to compounds of formula IIg-IIj, R³ is cyclopropyl, cyclobutyl, cyclopentyl or cyclohexyl, substituted with one or more substituents independently selected from Me, Et, Cl, F, CN, OH, OMe, OEt, CF₃, CHF₂, OCF₃, i-Pr, i-Bu, and t-Bu.

In one particular embodiment, with respect to compounds of formula IIg-IIj, R³ is substituted or unsubstituted heterocycloalkyl.

In one particular embodiment, with respect to compounds of formula IIg-IIj, R³ is unsubstituted pyrrolidinyl, piperidinyl, morpholinyl, tetrahydropyranyl, or piperazinyl.

In one particular embodiment, with respect to compounds of formula IIg-IIj, R³ is pyrrolidinyl, piperidinyl, morpholinyl, tetrahydropyranyl, or piperazinyl, substituted with one or more substituents independently selected from halo, hydroxyl, C₁-C₆alkyl, alkoxy and haloC₁-C₆alkyl.

In one particular embodiment, with respect to compounds of formula IIg-IIj, R³ is pyrrolidinyl, piperidinyl, morpholinyl, or piperazinyl, substituted with one or more substituents independently selected from Me, Et, Cl, F, CN, OH, OMe, OEt, CF₃, CHF₂, OCF₃, i-Pr, i-Bu, and t-Bu.

In one particular embodiment, with respect to compounds of formula IIg-IIj, R³ is pyrrolidinyl, piperidinyl, morpholinyl, or piperazinyl, substituted with Me.

In one particular embodiment, with respect to compounds of formula IIg-IIj, R³ is pyrrolidinyl, piperidinyl, morpholinyl, or piperazinyl, unsubstituted or substituted with Me.

In one particular embodiment, with respect to compounds of formula IIg-IIj, R³ is substituted or unsubstituted aryl or heteroaryl.

In one particular embodiment, with respect to compounds of formula IIg-IIj, R³ is phenyl, unsubstituted or substituted with one or more substituents independently selected from halo, hydroxyl, amino, cyano, sulfo, substituted sulfanyl, substituted sulfinyl, substituted sulfonyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

In one particular embodiment, with respect to compounds of formula IIg-IIj, R³ is phenyl substituted with one or more substituents independently selected from Me, Et, Ph, Cl, F, Br, CN, OH, OMe, OEt, OPh, COPh, CF₃, CHF₂, OCF₃, i-Pr, i-Bu, t-Bu, SMe, CH═CH—CO₂H, SOMe, SO₂Me, SO₃H, SO₃Me, and pyridyl. In one particular embodiment, R³ is monosubstituted phenyl. In another embodiment R³ is disubstituted phenyl.

In one particular embodiment, with respect to compounds of formula IIg-IIj, R³ is Ph, 2-Cl-Ph, 2-F-Ph, 4-Cl-Ph, 4-F-Ph, 2,4-dichlorophenyl, 2,4-difluorophenyl, 4-OH-Ph, or 2-OH-Ph.

In one particular embodiment, with respect to compounds of formula IIg-IIj, R³ is Ph substituted with substituted or unsubstituted amino. In another particular embodiment, R³ is Ph substituted with NH₂.

In one particular embodiment, with respect to compounds of formula IIg-IIj, R³ is Ph, 2-Cl-Ph, 2-F-Ph, 4-Cl-Ph, 4-F-Ph, 2,4-dichlorophenyl, 2,4-difluorophenyl, 4-OH-Ph, or 2-OH-Ph. In another particular embodiment, R³ is 4-OH-Ph.

In one particular embodiment, with respect to compounds of formula IIg-IIj, R³ is Ph substituted with Cl. In another particular embodiment, R³ is 2-Cl-Ph, 3-Cl-Ph, or 4-Cl-Ph. In yet another particular embodiment, R³ is 2-Cl.

In one particular embodiment, with respect to compounds of formula IIg-IIj, R³ is Ph substituted with F. In another particular embodiment, R³ is 2-F-Ph, 3-F-Ph, or 4-F-Ph. In yet another particular embodiment, R³ is 2-F.

In one particular embodiment, with respect to compounds of formula IIg-IIj, R³ is Ph substituted with OCF₃. In another particular embodiment, R³ is 3-OCF₃-Ph. In yet another particular embodiment, R³ is 2-OCF₃-Ph.

In one particular embodiment, with respect to compounds of formula IIg-IIj, R³ is Ph substituted with CF₃. In another particular embodiment, R³ is 3-CF₃-Ph. In yet another particular embodiment, R³ is 2-CF₃-Ph or 4-CF₃-Ph.

In one particular embodiment, with respect to compounds of formula IIg-IIj, R³ is 2,4-difluorophenyl.

In one particular embodiment, with respect to compounds of formula IIg-IIj, R³ is Ph substituted with NO₂. In another particular embodiment, R³ is 2-NO₂-Ph, 3-NO₂-Ph, or 4-NO₂-Ph. In yet another particular embodiment, R³ is 2-NO₂Ph. In yet another particular embodiment, R³ is 2,4-di-NO₂Ph.

In one particular embodiment, with respect to compounds of formula IIg-IIj, R³ is pyridyl, unsubstituted or substituted with one or more substituents independently selected from halo, hydroxyl, amino, cyano, sulfo, substituted sulfanyl, substituted sulfinyl, substituted sulfonyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

In one particular embodiment, with respect to compounds of formula IIg-IIj, R³ is pyridyl substituted with one or more substituents independently selected from Me, Et, Ph, Cl, F, Br, CN, OH, OMe, OEt, OPh, COPh, CF₃, CHF₂, OCF₃, i-Pr, i-Bu, t-Bu, SMe, CH═CH—CO₂H, SOMe, SO₂Me, SO₃H, SO₃Me, and pyridyl.

In one particular embodiment, with respect to compounds of formula IIg-IIj, R³ is unsubstituted pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl.

In one particular embodiment, with respect to compounds of formula IIg-IIj, R³ is unsubstituted indolyl, indazolyl, thiadiazolyl, or furanyl.

In one particular embodiment, with respect to compounds of formula IIg-IIj, R³ is pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl, substituted with one or more substituents independently selected from halo, hydroxyl, amino, cyano, sulfo, substituted sulfonyl, substituted sulfanyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

In one particular embodiment, with respect to compounds of formula IIg-IIj, R³ is pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl, substituted with one or more substituents independently selected from Me, Et, Ph, Cl, F, CN, OH, OMe, OEt, OPh, COPh, CF₃, CHF₂, OCF₃, i-Pr, i-Bu, t-Bu, SMe, CO₂Me, CO₂Et, CH═CH—CO₂H, SOMe, SO₂Me, SO₃H, SO₃Me, and pyridyl.

In one particular embodiment, with respect to compounds of formula IIg-IIj, R³ is unsubstituted thienyl, furanyl, pyrrolyl, pyrazolyl, imidazolyl, thiazolyl, thiadiazolyl, or oxadiazolyl.

In one particular embodiment, with respect to compounds of formula IIg-IIj, R³ is thienyl, furanyl, pyrrolyl, pyrazolyl, imidazolyl, thiazolyl, thiadiazolyl, or oxadiazolyl, substituted with one or more substituents independently selected from Me, Et, Cl, CF₃, CO₂Me, CO₂Et, and NHAc.

In one particular embodiment, with respect to compounds of formula IIg-IIj, R³ is Ph, thienyl, furanyl, pyrrolyl, pyrazolyl, imidazolyl, thiazolyl, thiadiazolyl, or oxadiazolyl, substituted with 5-membered heteroaryl. In one particular embodiment, the 5-membered heteroaryl is selected from pyrrolyl, thiophenyl, oxazolyl, pyrazolyl, thiazolyl, and thiadiazolyl. In another particular embodiment, the 5-membered heteroaryl substituted with halo or C₁-C₆ alkyl. In yet another particular embodiment, the 5-membered heteroaryl is selected from pyrrolyl, thiophenyl, oxazolyl, pyrazolyl, thiazolyl, and thiadiazolyl, substituted with one or more substituents independently selected from Me, Et, Cl, and CF₃.

In one particular embodiment, with respect to compounds of formula IIg-IIj, R³ is thiophenyl, methylthiophenyl, furanyl, methylfuranyl, pyrazolyl, or methylpyrazolyl.

In one particular embodiment, with respect to compounds of formula IIg-IIj, R³ is thiadiazolyl substituted with Me, Et, Cl, or CF₃. In another particular embodiment, R³ is thiadiazolyl substituted with Cl. In one particular embodiment, R³ is 1,2,4-thiadiazolyl.

In one particular embodiment, with respect to compounds of formula IIg-IIj, R³ is oxadiazolyl substituted with Me, Et, Cl, CO₂Et, or CF₃. In another particular embodiment, R³ is oxadiazolyl substituted with CO₂Et. In one particular embodiment, R³ is 1,2,4-oxadiazolyl.

In one particular embodiment, with respect to compounds of formula IIg-IIj, R³ is furanyl, unsubstituted or substituted with Me, Et, Cl, or CF₃. In another particular embodiment, R³ is unsubstituted furanyl. In yet another particular embodiment, R³ is furanyl substituted with Cl.

In one particular embodiment, with respect to compounds of formula IIg-IIj, R³ is thiophenyl, unsubstituted or substituted with Me, Et, Cl, CO₂Et, or CF₃. In another particular embodiment, R³ is unsubstituted thiophenyl. In yet another particular embodiment, R³ is thiophenyl substituted with C₁ or CO₂Et.

In one particular embodiment, with respect to compounds of formula IIg-IIj, R³ is oxazoyl or thiazolyl, unsubstituted or substituted with Me, Et, Cl, or CF₃. In another particular embodiment, R³ is unsubstituted thiazolyl. In yet another particular embodiment, R³ is thiazoyl substituted with Me or Cl. In yet another particular embodiment, R³ is thiazoyl or oxazolyl substituted with dimethyl.

In one particular embodiment, with respect to compounds of formula IIg-IIj, R³ is pyrazolyl substituted with Me, Et, Cl, CO₂Et, or CF₃. In another particular embodiment, R³ is pyrazolyl substituted with CO₂Et or Me.

In one particular embodiment, with respect to compounds of formula IIg-IIj, R³ is thiophenyl, methylthiophenyl, furanyl, methylfuranyl, pyrazolyl, or methylpyrazolyl, substituted with one or more substituents independently selected from Me, Et, Cl, CF₃, CO₂Me, CO₂Et, and NHAc.

In one particular embodiment, with respect to compounds of formula IIg-IIj, R³ is thiazolyl, pyridyl, cyclohexyl, phenyl, cyclopentyl, tetrahydrothiopyranyl, pyrazolyl, tetrahydropyranyl, unsubstituted or substituted with one or two Me.

In one particular embodiment, with respect to compounds of formula IIg-IIj, R³ is unsubstituted thiazolyl, pyridyl, cyclohexyl, phenyl, cyclopentyl, tetrahydrothiopyranyl, pyrazolyl, tetrahydropyranyl.

In one particular embodiment, with respect to compounds of formula IIg-IIj, R³ is thiazolyl, pyridyl, phenyl, or pyrazolyl, substituted with Me.

In one particular embodiment, with respect to compounds of formula IIg-IIj, R³ is thiazolyl, or pyrazolyl, substituted with diMe.

In one particular embodiment, with respect to compounds of formula IIg-IIj, R³ is dimethylthiazolyl, or dimethylpyrazolyl.

In some embodiments, R³ is a phenyl. In certain embodiments, R³ is a substituted phenyl.

In some embodiments, R³ is a mono-substituted phenyl.

In other embodiments, R³ is a di-substituted phenyl.

In certain embodiments, R³ is a substituted phenyl where the substituent on the phenyl is selected from halo, amido, C₁-C₆alkyl, alkoxy, sulfonyl, sulfonamidyl, haloalkyl and trihaloalkyl. In preferred embodiments, the substitution on the R³ phenyl is selected from Cl, F, CF₃, Me, t-Bu, OMe, SO₂R^{2′}, NR^{2′}R^{2′}, and SO₂NR^{2′}R^{2′}. In another embodiment, the substitution on the R³ phenyl is selected from Cl, Me, t-Bu and SO₂Me.

In embodiments where R³ is a substituted phenyl, one or more substituents are on the phenyl at the 2 (ortho), 3 (meta) and/or 4 (para) position relative to the carbon attached to the nitrogen atom in the fused heterocyclic scaffold in formula I. In certain embodiments, R³ is a substituted phenyl, where a substituent is on the phenyl at the 2 (ortho), 3 (meta) and/or 4 (para) position. In more preferred embodiments, the substitution on the R³ phenyl is at the 2 or 4 position. In the most preferred embodiments, the substitution on the R³ phenyl is at the 4 position.

In some embodiments, R³ are selected from

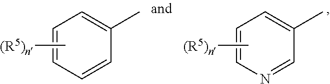

wherein subscript n′ is selected from 1-5 and each of R⁵ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol.

In some embodiments, subscript n′ is 1, 2 or 3.

In some embodiments, subscript n′ is 1 or 2.

In some embodiments, each R⁵ is independently selected from Me, Et, Pr, iso-Pr, Ph, Cl, F, Br, CN, OH, OMe, OEt, OPh, COPh, CO₂Me, CH₂—N-morpholino, CH₂—N-(4-Me-piperidino), CONH₂, CF₃, CHF₂, OCF₃, OCHF₂, t-Bu, SMe, CH═CH—CO₂H, SOMe, SO₂Me, SO₂CF₃, SO₂NH₂, SO₃H, SO₃Me, and pyridyl.

In some embodiments, each R⁵ is independently selected from Me, Et, Pr, iso-Pr, Ph, Cl, F, CN, OH, OMe, OEt, OPh, CF₃, CHF₂, OCF₃, OCHF₂, t-Bu, SO₂Me, SO₂CF₃, and SO₃Me.

In one particular embodiment, with respect to compounds of formula I, the compound is according to formula IIIa, IIIb, IIIc, IIId, IIIe, IIIf, IIIg, IIIh or IIIi:

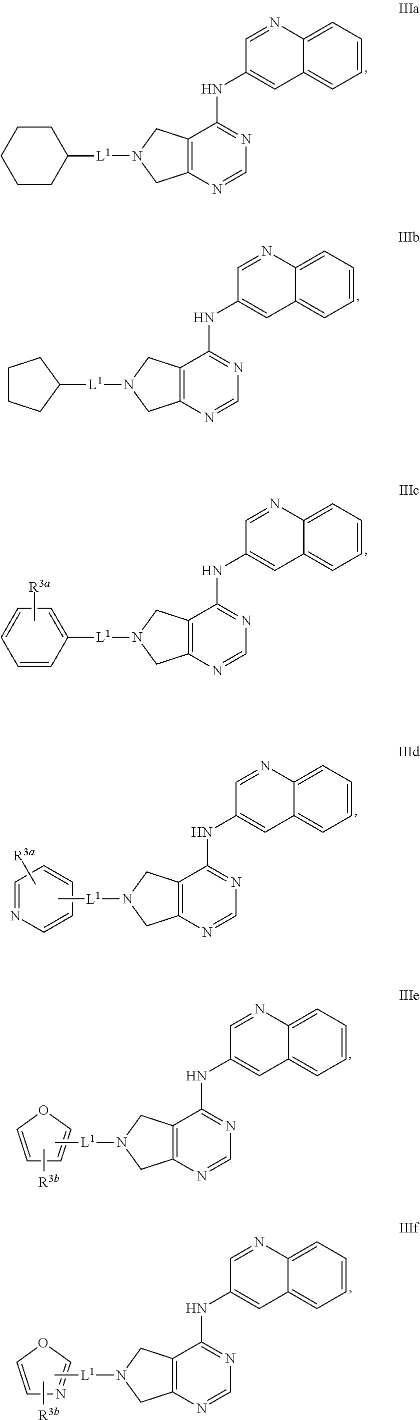

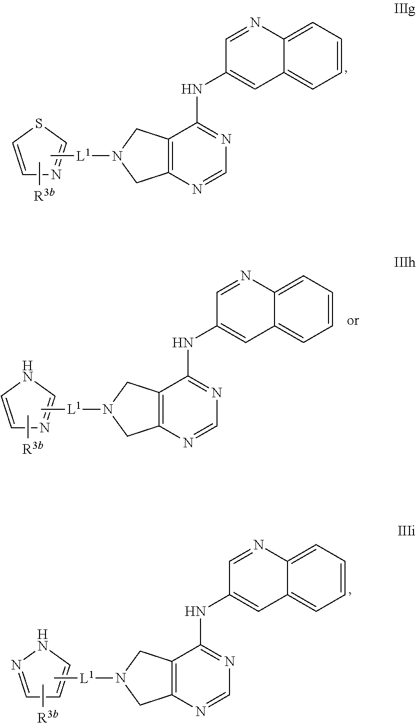

and wherein L¹ is a single bond, —CH₂—, —CO—, —NHC(O)—, —OC(O)—, or S(O)₂; and R^{3a} is H, alkyl, alkoxy, halo, haloalkyl, thioalkyl, or haloalkyloxy; and R^{3b} is H or alkyl.

In one particular embodiment, with respect to compounds of formula IIIa-IIIi, the compound is according to formula IIIa or IIIb.

In one particular embodiment, with respect to compounds of formula IIIa-IIIi, the compound is according to formula IIIc.

In one particular embodiment, with respect to compounds of formula IIIa-IIIi, the compound is according to formula IIId.

In one particular embodiment, with respect to compounds of formula IIIa-IIIi, the compound is according to formula IIIe.

In one particular embodiment, with respect to compounds of formula IIIa-IIIi, the compound is according to formula IIIf.

In one particular embodiment, with respect to compounds of formula IIIa-IIIi, the compound is according to formula IIIg.

In one particular embodiment, with respect to compounds of formula IIIa-IIIi, the compound is according to formula IIIh.

In one particular embodiment, with respect to compounds of formula IIIa-IIIi, the compound is according to formula IIIi.

In one particular embodiment, with respect to compounds of formula IIIa-IIIi, R^{3a} is H, Me, Et, OMe, CF₃, OCF₃, OCF₂, SMe, Cl, or F. In another embodiment R^{3a} is H.

In one particular embodiment, with respect to compounds of formula IIIa-IIIi, R^{3b} is H, Me, Et, or i-Pr. In another embodiment R^{3b} is H.

In one particular embodiment, with respect to compounds of formula IIIa-IIIi, L¹ is a single bond or —CH₂—.

In one particular embodiment, with respect to compounds of formula IIIa-IIIi, L¹ is a single bond.

In one particular embodiment, with respect to compounds of formula IIIa-IIIi, L¹ is —CH₂—.

In one particular embodiment, with respect to compounds of formula IIIa-IIIi, L¹ is —CO—, —NHC(O)—, —OC(O)—, or S(O)₂.

In one particular embodiment, with respect to compounds of formula IIIa-IIIi, L¹ is —CO—, —NHC(O)—, or —OC(O)—.

In one particular embodiment, with respect to compounds of formula IIIa-IIIi, L¹ is —S(O)₂—.

In one particular embodiment, with respect to compounds of formula I, the compound is according to formula IVa, IVb, IVc, IVd, IVe, IVf, IVg, IVh or IVi:

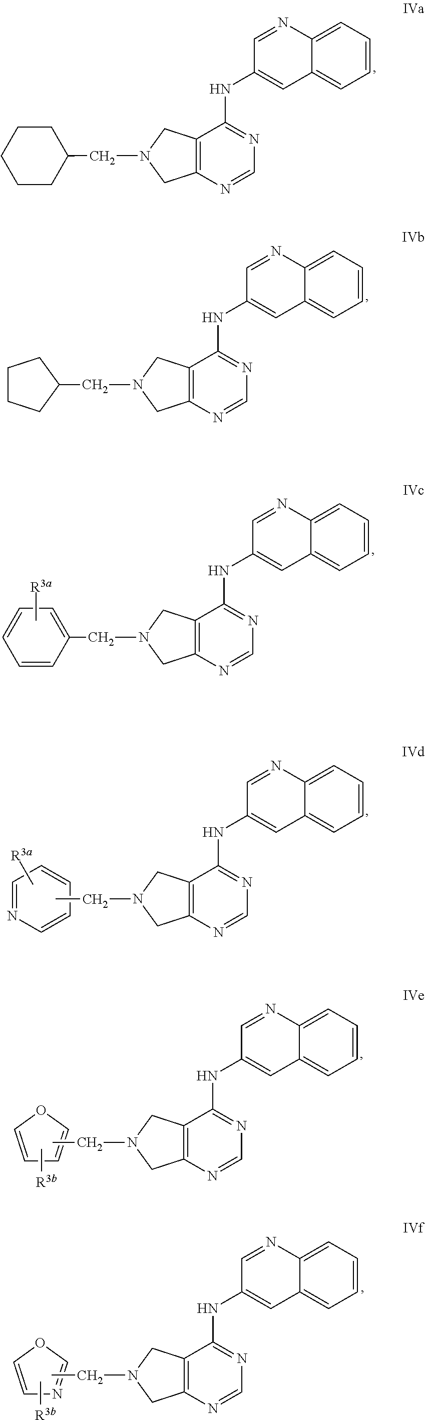

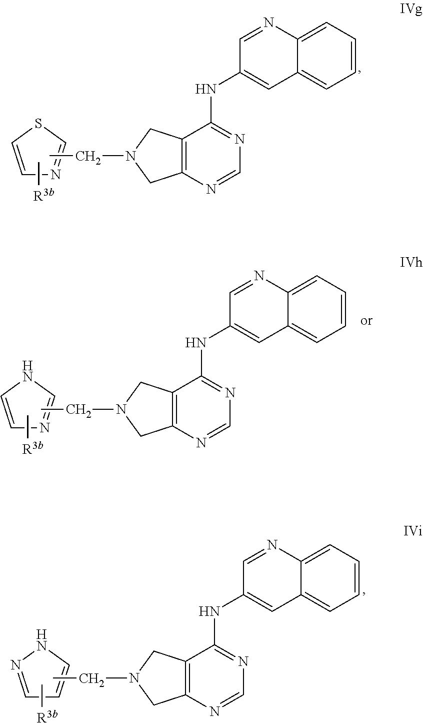

and wherein R^{3a} is H, alkyl, alkoxy, halo, haloalkyl, thioalkyl, or haloalkyloxy; and R^{3b} is H or alkyl.

In one particular embodiment, with respect to compounds of formula I, the compound is according to formula Va, Vb, Vc, Vd, Ve, Vf, Vg, Vh or Vi:

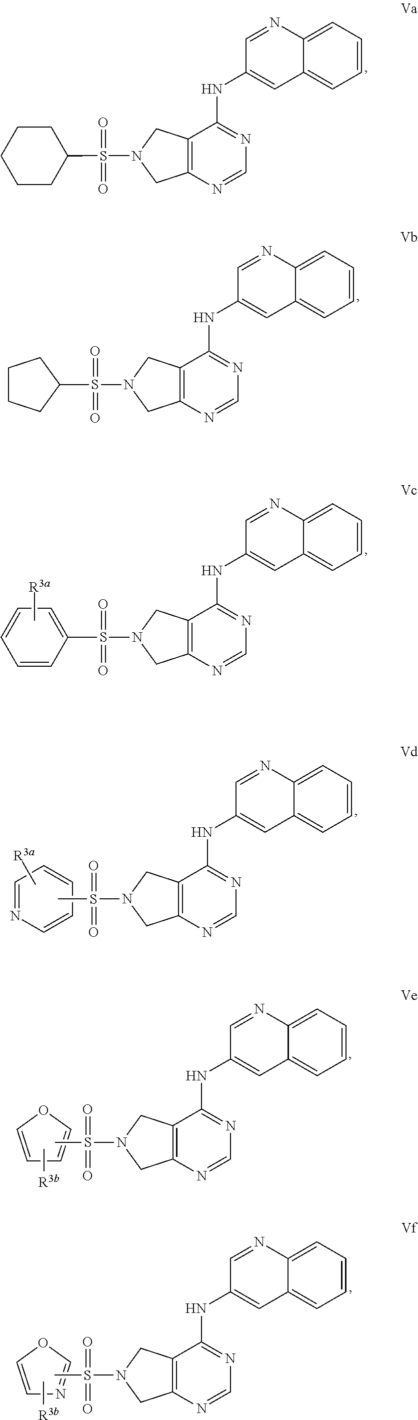

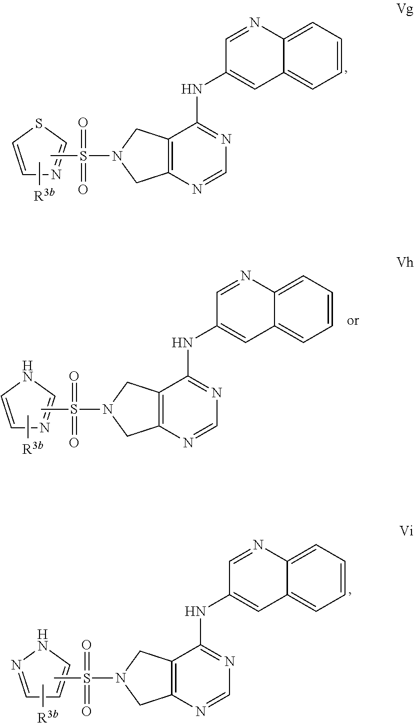

and wherein R^{3a} is H, alkyl, alkoxy, halo, haloalkyl, thioalkyl, or haloalkyloxy; and R^{3b} is H or alkyl.

In one particular embodiment, with respect to compounds of formula IVa-Vi, R^{3a} is H, Me, Et, OMe, CF₃, OCF₃, OCF₂, SMe, Cl, or F. In another embodiment R^{3a} is H.

In one particular embodiment, with respect to compounds of formula IVa-Vi, R^{3b} is H, Me, Et, or i-Pr. In another embodiment R^{3b} is H.

In one particular embodiment, with respect to compounds of formula IVa-Vi, each of R^{3a} and R^{3b} is H,

With regard to compounds of formula I, in certain embodiments, the compound is selected from the compounds listed in Table 1.

With regard to compounds of formula I, in certain embodiments, the compound is selected from:
(6-Benzyl-6,7-dihydro-5H-pyrrolo[3,4-d]pyrimidin-4-yl)-quinolin-3-yl-amine;
(6-Cyclohexylmethyl-6,7-dihydro-5H-pyrrolo[3,4-d]pyrimidin-4-yl)-quinolin-3-yl-amine;
(6-Cyclopentylmethyl-6,7-dihydro-5H-pyrrolo[3,4-d]pyrimidin-4-yl)-quinolin-3-yl-amine;
[6-(2-Ethyl-butyl)-6,7-dihydro-5H-pyrrolo[3,4-d]pyrimidin-4-yl]-quinolin-3-yl-amine;
[6-(2-Methyl-pyridin-3-ylmethyl)-6,7-dihydro-5H-pyrrolo[3,4-d]pyrimidin-4-yl]-quinolin-3-yl-amine;
Quinolin-3-yl-[6-(tetrahydro-thiopyran-4-ylmethyl)-6,7-dihydro-5H-pyrrolo[3,4-d]pyrimidin-4-yl]-amine;
[6-(2-Phenyl-propyl)-6,7-dihydro-5H-pyrrolo[3,4-d]pyrimidin-4-yl]-quinolin-3-yl-amine;
(6-Phenethyl-6,7-dihydro-5H-pyrrolo[3,4-d]pyrimidin-4-yl)-quinolin-3-yl-amine;
[6-(2,4-Dimethyl-thiazol-5-ylmethyl)-6,7-dihydro-5H-pyrrolo[3,4-d]pyrimidin-4-yl]-quinolin-3-yl-amine;
Quinolin-3-yl-[6-(tetrahydro-pyran-4-ylmethyl)-6,7-dihydro-5H-pyrrolo[3,4-d]pyrimidin-4-yl]-amine;
[6-(1-Cyclohexyl-ethyl)-6,7-dihydro-5H-pyrrolo[3,4-d]pyrimidin-4-yl]-quinolin-3-yl-amine;
Quinolin-3-yl-[6-(toluene-2-sulfonyl)-6,7-dihydro-5H-pyrrolo[3,4-d]pyrimidin-4-yl]-amine;
[6-(4-Methyl-thiazol-5-ylmethyl)-6,7-dihydro-5H-pyrrolo[3,4-d]pyrimidin-4-yl]-quinolin-3-yl-amine;
5-[4-(Quinolin-3-ylamino)-5,7-dihydro-pyrrolo[3,4-d]pyrimidine-6-sulfonyl]-furan-2-carboxylic acid methyl ester;
[6-(3-Methyl-pyridin-4-ylmethyl)-6,7-dihydro-5H-pyrrolo[3,4-d]pyrimidin-4-yl]-quinolin-3-yl-amine;
[6-(2-Fluoro-benzyl)-6,7-dihydro-5H-pyrrolo[3,4-d]pyrimidin-4-yl]-quinolin-3-yl-amine;
[6-(2-Methoxy-benzyl)-6,7-dihydro-5H-pyrrolo[3,4-d]pyrimidin-4-yl]-quinolin-3-yl-amine;
[6-(2-Chloro-benzyl)-6,7-dihydro-5H-pyrrolo[3,4-d]pyrimidin-4-yl]-quinolin-3-yl-amine;
[6-(3-Methyl-furan-2-ylmethyl)-6,7-dihydro-5H-pyrrolo[3,4-d]pyrimidin-4-yl]-quinolin-3-yl-amine;
[6-(2,3-Dimethyl-benzyl)-6,7-dihydro-5H-pyrrolo[3,4-d]pyrimidin-4-yl]-quinolin-3-yl-amine;
[6-(2,5-Dimethyl-benzyl)-6,7-dihydro-5H-pyrrolo[3,4-d]pyrimidin-4-yl]-quinolin-3-yl-amine;
[6-(2-Ethyl-benzyl)-6,7-dihydro-5H-pyrrolo[3,4-d]pyrimidin-4-yl]-quinolin-3-yl-amine;
[6-(2-Methyl-benzyl)-6,7-dihydro-5H-pyrrolo[3,4-d]pyrimidin-4-yl]-quinolin-3-yl-amine;
(6-Pyridin-4-ylmethyl-6,7-dihydro-5H-pyrrolo[3,4-d]pyrimidin-4-yl)-quinolin-3-yl-amine;
Quinolin-3-yl-[6-(2-trifluoromethyl-benzyl)-6,7-dihydro-5H-pyrrolo[3,4-d]pyrimidin-4-yl]-amine;
[6-(2-Ethyl-4-methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzyl)-6,7-dihydro-5H-pyrrolo[3,4-d]pyrimidin-4-yl]-amine;
Quinolin-3-yl-[6-(3-trifluoromethyl-benzyl)-6,7-dihydro-5H-pyrrolo[3,4-d]pyrimidin-4-yl]-amine;
[6-(2-Ethoxy-benzyl)-6,7-dihydro-5H-pyrrolo[3,4-d]pyrimidin-4-yl]-quinolin-3-yl-amine;
[6-(2,3-Dihydro-benzofuran-7-ylmethyl)-6,7-dihydro-5H-pyrrolo[3,4-d]pyrimidin-4-yl]-quinolin-3-yl-amine;
[6-(2-Methylsulfanyl-benzyl)-6,7-dihydro-5H-pyrrolo[3,4-d]pyrimidin-4-yl]-quinolin-3-yl-amine;
(6-Benzo[1,3]dioxol-4-ylmethyl-6,7-dihydro-5H-pyrrolo[3,4-d]pyrimidin-4-yl)-quinolin-3-yl-amine;
Quinolin-3-yl-[6-(3-trifluoromethoxy-benzyl)-6,7-dihydro-5H-pyrrolo[3,4-d]pyrimidin-4-yl]-amine;
[6-(2-Difluoromethoxy-benzyl)-6,7-dihydro-5H-pyrrolo[3,4-d]pyrimidin-4-yl]-quinolin-3-yl-amine;
(6-Benzyl-6,7-dihydro-5H-pyrrolo[3,4-d]pyrimidin-4-yl)-quinolin-3-yl-amine;
(6-Cyclohexylmethyl-6,7-dihydro-5H-pyrrolo[3,4-d]pyrimidin-4-yl)-quinolin-3-yl-amine;
(6-Ethyl-6,7-dihydro-5H-pyrrolo[3,4-d]pyrimidin-4-yl)-quinolin-3-yl-amine;
(6-Propyl-6,7-dihydro-5H-pyrrolo[3,4-d]pyrimidin-4-yl)-quinolin-3-yl-amine;
(6-Cyclopropylmethyl-6,7-dihydro-5H-pyrrolo[3,4-d]pyrimidin-4-yl)-quinolin-3-yl-amine;
(6-Butyl-6,7-dihydro-5H-pyrrolo[3,4-d]pyrimidin-4-yl)-quinolin-3-yl-amine;
(6-Isobutyl-6,7-dihydro-5H-pyrrolo[3,4-d]pyrimidin-4-yl)-quinolin-3-yl-amine;
[6-(2-Methyl-butyl)-6,7-dihydro-5H-pyrrolo[3,4-d]pyrimidin-4-yl]-quinolin-3-yl-amine;
[6-(3-Methyl-butyl)-6,7-dihydro-5H-pyrrolo[3,4-d]pyrimidin-4-yl]-quinolin-3-yl-amine;
(6-Pentyl-6,7-dihydro-5H-pyrrolo[3,4-d]pyrimidin-4-yl)-quinolin-3-yl-amine;
[6-(2,2-Dimethyl-propyl)-6,7-dihydro-5H-pyrrolo[3,4-d]pyrimidin-4-yl]-quinolin-3-yl-amine;
(6-Cyclopentylmethyl-6,7-dihydro-5H-pyrrolo[3,4-d]pyrimidin-4-yl)-quinolin-3-yl-amine;
Quinolin-3-yl-[6-(tetrahydro-furan-3-ylmethyl)-6,7-dihydro-5H-pyrrolo[3,4-d]pyrimidin-4-yl]-amine;
[6-(2-Ethyl-butyl)-6,7-dihydro-5H-pyrrolo[3,4-d]pyrimidin-4-yl]-quinolin-3-yl-amine;
[6-(3,3-Dimethyl-butyl)-6,7-dihydro-5H-pyrrolo[3,4-d]pyrimidin-4-yl]-quinolin-3-yl-amine;
Quinolin-3-yl-[6-(tetrahydro-pyran-3-ylmethyl)-6,7-dihydro-5H-pyrrolo[3,4-d]pyrimidin-4-yl]-amine;
[6-(2-Methyl-pyridin-3-ylmethyl)-6,7-dihydro-5H-pyrrolo[3,4-d]pyrimidin-4-yl]-quinolin-3-yl-amine;
Quinolin-3-yl-[6-(tetrahydro-thiopyran-4-ylmethyl)-6,7-dihydro-5H-pyrrolo[3,4-d]pyrimidin-4-yl]-amine;
[6-(2-Phenyl-propyl)-6,7-dihydro-5H-pyrrolo[3,4-d]pyrimidin-4-yl]-quinolin-3-yl-amine;
4-[4-(Quinolin-3-ylamino)-5,7-dihydro-pyrrolo[3,4-d]pyrimidin-6-yl]-butan-1-ol;
(6-Cyclobutylmethyl-6,7-dihydro-5H-pyrrolo[3,4-d]pyrimidin-4-yl)-quinolin-3-yl-amine;
(6-Phenethyl-6,7-dihydro-5H-pyrrolo[3,4-d]pyrimidin-4-yl)-quinolin-3-yl-amine;
(6-Cyclohexyl-6,7-dihydro-5H-pyrrolo[3,4-d]pyrimidin-4-yl)-quinolin-3-yl-amine;
[6-(2-Methyl-2H-pyrazol-3-ylmethyl)-6,7-dihydro-5H-pyrrolo[3,4-d]pyrimidin-4-yl]-quinolin-3-yl-amine;
[6-(2-Methyl-thiazol-4-ylmethyl)-6,7-dihydro-5H-pyrrolo[3,4-d]pyrimidin-4-yl]-quinolin-3-yl-amine;
[6-(1,5-Dimethyl-1H-pyrazol-4-ylmethyl)-6,7-dihydro-5H-pyrrolo[3,4-d]pyrimidin-4-yl]-quinolin-3-yl-amine;
[6-(1-Methyl-piperidin-4-ylmethyl)-6,7-dihydro-5H-pyrrolo[3,4-d]pyrimidin-4-yl]-quinolin-3-yl-amine;
[6-(2,4-Dimethyl-thiazol-5-ylmethyl)-6,7-dihydro-5H-pyrrolo[3,4-d]pyrimidin-4-yl]-quinolin-3-yl-amine;
[6-(3-Methyl-oxetan-3-ylmethyl)-6,7-dihydro-5H-pyrrolo[3,4-d]pyrimidin-4-yl]-quinolin-3-yl-amine;
Quinolin-3-yl-[6-(2,2,2-trifluoro-ethyl)-6,7-dihydro-5H-pyrrolo[3,4-d]pyrimidin-4-yl]-amine;
Quinolin-3-yl-[6-(3,3,3-trifluoro-propyl)-6,7-dihydro-5H-pyrrolo[3,4-d]pyrimidin-4-yl]-amine;
[6-(1,2-Dimethyl-propyl)-6,7-dihydro-5H-pyrrolo[3,4-d]pyrimidin-4-yl]-quinolin-3-yl-amine;
Quinolin-3-yl-[6-(tetrahydro-pyran-4-ylmethyl)-6,7-dihydro-5H-pyrrolo[3,4-d]pyrimidin-4-yl]-amine;
(6-Isopropyl-6,7-dihydro-5H-pyrrolo[3,4-d]pyrimidin-4-yl)-quinolin-3-yl-amine;
(6-sec-Butyl-6,7-dihydro-5H-pyrrolo[3,4-d]pyrimidin-4-yl)-quinolin-3-yl-amine;
[6-(1-Cyclobutyl-ethyl)-6,7-dihydro-5H-pyrrolo[3,4-d]pyrimidin-4-yl]-quinolin-3-yl-amine;
[6-(1-Cyclopentyl-ethyl)-6,7-dihydro-5H-pyrrolo[3,4-d]pyrimidin-4-yl]-quinolin-3-yl-amine;
[6-(1-Cyclohexyl-ethyl)-6,7-dihydro-5H-pyrrolo[3,4-d]pyrimidin-4-yl]-quinolin-3-yl-amine;
Quinolin-3-yl-[6-(1,3,3-trimethyl-butyl)-6,7-dihydro-5H-pyrrolo[3,4-d]pyrimidin-4-yl]-amine;
(6-Cycloheptyl-6,7-dihydro-5H-pyrrolo[3,4-d]pyrimidin-4-yl)-quinolin-3-yl-amine;
(6-Cyclopentyl-6,7-dihydro-5H-pyrrolo[3,4-d]pyrimidin-4-yl)-quinolin-3-yl-amine;
[6-(1-Methyl-butyl)-6,7-dihydro-5H-pyrrolo[3,4-d]pyrimidin-4-yl]-quinolin-3-yl-amine;
(6-Cyclobutyl-6,7-dihydro-5H-pyrrolo[3,4-d]pyrimidin-4-yl)-quinolin-3-yl-amine;
(6-Oxetan-3-yl-6,7-dihydro-5H-pyrrolo[3,4-d]pyrimidin-4-yl)-quinolin-3-yl-amine;
4-(Quinolin-3-ylamino)-5,7-dihydro-pyrrolo[3,4-d]pyrimidine-6-carboxylic acid tert-butyl ester;
4-(Quinolin-3-ylamino)-5,7-dihydro-pyrrolo[3,4-d]pyrimidine-6-carboxylic acid cyclohexylmethyl-amide;
4-(Quinolin-3-ylamino)-5,7-dihydro-pyrrolo[3,4-d]pyrimidine-6-carboxylic acid cyclohexylamide;
4-(Quinolin-3-ylamino)-5,7-dihydro-pyrrolo[3,4-d]pyrimidine-6-carboxylic acid (2,4-dimethyl-thiazol-5-ylmethyl)-amide;
(2,4-Dimethyl-thiazol-5-yl)-[4-(quinolin-3-ylamino)-5,7-dihydro-pyrrolo[3,4-d]pyrimidin-6-yl]-methanone;
2-(2,4-Dimethyl-thiazol-5-yl)-1-[4-(quinolin-3-ylamino)-5,7-dihydro-pyrrolo[3,4-d]pyrimidin-6-yl]-ethanone;
4-(Quinolin-3-ylamino)-5,7-dihydro-pyrrolo[3,4-d]pyrimidine-6-carboxylic acid (2,4-dimethyl-thiazol-5-yl)-amide;
2-Azepan-1-yl-1-[4-(quinolin-3-ylamino)-5,7-dihydro-pyrrolo[3,4-d]pyrimidin-6-yl]-ethanone;
2-Piperidin-1-yl-1-[4-(quinolin-3-ylamino)-5,7-dihydro-pyrrolo[3,4-d]pyrimidin-6-yl]-ethanone;
2-Pyrrolidin-1-yl-1-[4-(quinolin-3-ylamino)-5,7-dihydro-pyrrolo[3,4-d]pyrimidin-6-yl]-ethanone;
[6-(5-Oxazol-5-yl-thiophene-2-sulfonyl)-6,7-dihydro-5H-pyrrolo[3,4-d]pyrimidin-4-yl]-quinolin-3-yl-amine;
[6-(3-Methyl-thiophene-2-sulfonyl)-6,7-dihydro-5H-pyrrolo[3,4-d]pyrimidin-4-yl]-quinolin-3-yl-amine;
(6-Cyclopentanesulfonyl-6,7-dihydro-5H-pyrrolo[3,4-d]pyrimidin-4-yl)-quinolin-3-yl-amine;
[6-(2,4-Difluoro-benzenesulfonyl)-6,7-dihydro-5H-pyrrolo[3,4-d]pyrimidin-4-yl]-quinolin-3-yl-amine;
Quinolin-3-yl-[6-(toluene-2-sulfonyl)-6,7-dihydro-5H-pyrrolo[3,4-d]pyrimidin-4-yl]-amine;
[6-(2-Methyl-2H-pyrazole-3-sulfonyl)-6,7-dihydro-5H-pyrrolo[3,4-d]pyrimidin-4-yl]-quinolin-3-yl-amine;
[6-(2,4-Dimethyl-thiazole-5-sulfonyl)-6,7-dihydro-5H-pyrrolo[3,4-d]pyrimidin-4-yl]-quinolin-3-yl-amine;
2-Methyl-5-[4-(quinolin-3-ylamino)-5,7-dihydro-pyrrolo[3,4-d]pyrimidine-6-sulfonyl]-furan-3-carboxylic acid methyl ester;
(6-Phenylmethanesulfonyl-6,7-dihydro-5H-pyrrolo[3,4-d]pyrimidin-4-yl)-quinolin-3-yl-amine;
(6-Cyclohexanesulfonyl-6,7-dihydro-5H-pyrrolo[3,4-d]pyrimidin-4-yl)-quinolin-3-yl-amine;
(6-Benzenesulfonyl-6,7-dihydro-5H-pyrrolo[3,4-d]pyrimidin-4-yl)-quinolin-3-yl-amine;
[6-(4-Methyl-thiazol-5-ylmethyl)-6,7-dihydro-5H-pyrrolo[3,4-d]pyrimidin-4-yl]-quinolin-3-yl-amine;
[6-(3,5-Dimethyl-isoxazol-4-ylmethyl)-6,7-dihydro-5H-pyrrolo[3,4-d]pyrimidin-4-yl]-quinolin-3-yl-amine;
5-[4-(Quinolin-3-ylamino)-5,7-dihydro-pyrrolo[3,4-d]pyrimidine-6-sulfonyl]-furan-2-carboxylic acid methyl ester;
[6-(2-Methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzenesulfonyl)-6,7-dihydro-5H-pyrrolo[3,4-d]pyrimidin-4-yl]-amine;
[6-(2-Fluoro-benzenesulfonyl)-6,7-dihydro-5H-pyrrolo[3,4-d]pyrimidin-4-yl]-quinolin-3-yl-amine;
[6-(2-Chloro-benzenesulfonyl)-6,7-dihydro-5H-pyrrolo[3,4-d]pyrimidin-4-yl]-quinolin-3-yl-amine;
[6-(2-Methoxy-benzenesulfonyl)-6,7-dihydro-5H-pyrrolo[3,4-d]pyrimidin-4-yl]-quinolin-3-yl-amine;
[6-(2-Methyl-6-trifluoromethyl-pyridin-3-ylmethyl)-6,7-dihydro-5H-pyrrolo[3,4-d]pyrimidin-4-yl]-quinolin-3-yl-amine;
Quinolin-3-yl-(6-thiazol-4-ylmethyl-6,7-dihydro-5H-pyrrolo[3,4-d]pyrimidin-4-yl)-amine;
Quinolin-3-yl-(6-thiazol-2-ylmethyl-6,7-dihydro-5H-pyrrolo[3,4-d]pyrimidin-4-yl)-amine;
[6-(4-Methyl-thiazol-2-ylmethyl)-6,7-dihydro-5H-pyrrolo[3,4-d]pyrimidin-4-yl]-quinolin-3-yl-amine;
[6-(3-Methyl-pyridin-4-ylmethyl)-6,7-dihydro-5H-pyrrolo[3,4-d]pyrimidin-4-yl]-quinolin-3-yl-amine;
[6-(3-Methyl-pyridin-2-ylmethyl)-6,7-dihydro-5H-pyrrolo[3,4-d]pyrimidin-4-yl]-quinolin-3-yl-amine;
[6-(2-Fluoro-benzyl)-6,7-dihydro-5H-pyrrolo[3,4-d]pyrimidin-4-yl]-quinolin-3-yl-amine;
Quinolin-3-yl-(6-thiazol-5-ylmethyl-6,7-dihydro-5H-pyrrolo[3,4-d]pyrimidin-4-yl)-amine;
[6-(2-Phenyl-ethanesulfonyl)-6,7-dihydro-5H-pyrrolo[3,4-d]pyrimidin-4-yl]-quinolin-3-yl-amine;
[6-(2-Methoxy-benzyl)-6,7-dihydro-5H-pyrrolo[3,4-d]pyrimidin-4-yl]-quinolin-3-yl-amine;
[6-(2-Chloro-benzyl)-6,7-dihydro-5H-pyrrolo[3,4-d]pyrimidin-4-yl]-quinolin-3-yl-amine;
Quinolin-3-yl-(6-quinolin-4-ylmethyl-6,7-dihydro-5H-pyrrolo[3,4-d]pyrimidin-4-yl)-amine;
[6-(3-Methyl-furan-2-ylmethyl)-6,7-dihydro-5H-pyrrolo[3,4-d]pyrimidin-4-yl]-quinolin-3-yl-amine;
[6-(2,3-Dimethyl-benzyl)-6,7-dihydro-5H-pyrrolo[3,4-d]pyrimidin-4-yl]-quinolin-3-yl-amine;
[6-(2,6-Dimethyl-benzyl)-6,7-dihydro-5H-pyrrolo[3,4-d]pyrimidin-4-yl]-quinolin-3-yl-amine;
[6-(2,5-Dimethyl-benzyl)-6,7-dihydro-5H-pyrrolo[3,4-d]pyrimidin-4-yl]-quinolin-3-yl-amine;
[6-(2-Ethyl-benzyl)-6,7-dihydro-5H-pyrrolo[3,4-d]pyrimidin-4-yl]-quinolin-3-yl-amine;
[6-(2-Chloro-thiazol-5-ylmethyl)-6,7-dihydro-5H-pyrrolo[3,4-d]pyrimidin-4-yl]-quinolin-3-yl-amine;
[6-(2-Methyl-benzyl)-6,7-dihydro-5H-pyrrolo[3,4-d]pyrimidin-4-yl]-quinolin-3-yl-amine;
[6-(2,4-Dimethyl-benzyl)-6,7-dihydro-5H-pyrrolo[3,4-d]pyrimidin-4-yl]-quinolin-3-yl-amine;
(6-Pyridin-4-ylmethyl-6,7-dihydro-5H-pyrrolo[3,4-d]pyrimidin-4-yl)-quinolin-3-yl-amine;
Quinolin-3-yl-[6-(2-trifluoromethyl-benzyl)-6,7-dihydro-5H-pyrrolo[3,4-d]pyrimidin-4-yl]-amine;
[6-(2-Methyl-furan-3-ylmethyl)-6,7-dihydro-5H-pyrrolo[3,4-d]pyrimidin-4-yl]-quinolin-3-yl-amine;
[6-(2-Ethyl-4-methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzyl)-6,7-dihydro-5H-pyrrolo[3,4-d]pyrimidin-4-yl]-amine;
Quinolin-3-yl-[6-(3-trifluoromethyl-benzyl)-6,7-dihydro-5H-pyrrolo[3,4-d]pyrimidin-4-yl]-amine;
[6-(2-Ethoxy-benzyl)-6,7-dihydro-5H-pyrrolo[3,4-d]pyrimidin-4-yl]-quinolin-3-yl-amine;
[6-(2,3-Dihydro-benzofuran-7-ylmethyl)-6,7-dihydro-5H-pyrrolo[3,4-d]pyrimidin-4-yl]-quinolin-3-yl-amine;
[6-(2-Methylsulfanyl-benzyl)-6,7-dihydro-5H-pyrrolo[3,4-d]pyrimidin-4-yl]-quinolin-3-yl-amine;
(6-Benzo[1,3]dioxol-4-ylmethyl-6,7-dihydro-5H-pyrrolo[3,4-d]pyrimidin-4-yl)-quinolin-3-yl-amine;
Quinolin-3-yl-[6-(3-trifluoromethoxy-benzyl)-6,7-dihydro-5H-pyrrolo[3,4-d]pyrimidin-4-yl]-amine;
[6-(2-Difluoromethoxy-benzyl)-6,7-dihydro-5H-pyrrolo[3,4-d]pyrimidin-4-yl]-quinolin-3-yl-amine;
[6-(3-Methoxy-benzyl)-6,7-dihydro-5H-pyrrolo[3,4-d]pyrimidin-4-yl]-quinolin-3-yl-amine;
[6-(2,3-Difluoro-benzyl)-6,7-dihydro-5H-pyrrolo[3,4-d]pyrimidin-4-yl]-quinolin-3-yl-amine;
[6-(2-Propoxy-benzyl)-6,7-dihydro-5H-pyrrolo[3,4-d]pyrimidin-4-yl]-quinolin-3-yl-amine;
[6-(2-Isopropoxy-benzyl)-6,7-dihydro-5H-pyrrolo[3,4-d]pyrimidin-4-yl]-quinolin-3-yl-amine;
[6-(1H-Indol-7-ylmethyl)-6,7-dihydro-5H-pyrrolo[3,4-d]pyrimidin-4-yl]-quinolin-3-yl-amine;
[6-(3-Methyl-benzyl)-6,7-dihydro-5H-pyrrolo[3,4-d]pyrimidin-4-yl]-quinolin-3-yl-amine; and
[6-(2-Cyclopropyl-benzyl)-6,7-dihydro-5H-pyrrolo[3,4-d]pyrimidin-4-yl]-quinolin-3-yl-amine;

or a pharmaceutically acceptable salt, solvate or prodrug thereof;

and stereoisomers, isotopic variants and tautomers thereof.

With regard to compounds of formula I, in certain embodiments, the compound is selected from:
(6-Benzyl-6,7-dihydro-5H-pyrrolo[3,4-d]pyrimidin-4-yl)-quinolin-3-yl-amine;
(6-Cyclohexylmethyl-6,7-dihydro-5H-pyrrolo[3,4-d]pyrimidin-4-yl)-quinolin-3-yl-amine;
(6-Cyclopentylmethyl-6,7-dihydro-5H-pyrrolo[3,4-d]pyrimidin-4-yl)-quinolin-3-yl-amine;
[6-(2-Ethyl-butyl)-6,7-dihydro-5H-pyrrolo[3,4-d]pyrimidin-4-yl]-quinolin-3-yl-amine;
[6-(2-Methyl-pyridin-3-ylmethyl)-6,7-dihydro-5H-pyrrolo[3,4-d]pyrimidin-4-yl]-quinolin-3-yl-amine;
Quinolin-3-yl-[6-(tetrahydro-thiopyran-4-ylmethyl)-6,7-dihydro-5H-pyrrolo[3,4-d]pyrimidin-4-yl]-amine;
[6-(2-Phenyl-propyl)-6,7-dihydro-5H-pyrrolo[3,4-d]pyrimidin-4-yl]-quinolin-3-yl-amine;
(6-Phenethyl-6,7-dihydro-5H-pyrrolo[3,4-d]pyrimidin-4-yl)-quinolin-3-yl-amine;
[6-(2,4-Dimethyl-thiazol-5-ylmethyl)-6,7-dihydro-5H-pyrrolo[3,4-d]pyrimidin-4-yl]-quinolin-3-yl-amine;
Quinolin-3-yl-[6-(tetrahydro-pyran-4-ylmethyl)-6,7-dihydro-5H-pyrrolo[3,4-d]pyrimidin-4-yl]-amine;
[6-(1-Cyclohexyl-ethyl)-6,7-dihydro-5H-pyrrolo[3,4-d]pyrimidin-4-yl]-quinolin-3-yl-amine;
Quinolin-3-yl-[6-(toluene-2-sulfonyl)-6,7-dihydro-5H-pyrrolo[3,4-d]pyrimidin-4-yl]-amine;
[6-(4-Methyl-thiazol-5-ylmethyl)-6,7-dihydro-5H-pyrrolo[3,4-d]pyrimidin-4-yl]-quinolin-3-yl-amine;
5-[4-(Quinolin-3-ylamino)-5,7-dihydro-pyrrolo[3,4-d]pyrimidine-6-sulfonyl]-furan-2-carboxylic acid methyl ester;
[6-(3-Methyl-pyridin-4-ylmethyl)-6,7-dihydro-5H-pyrrolo[3,4-d]pyrimidin-4-yl]-quinolin-3-yl-amine;
[6-(2-Fluoro-benzyl)-6,7-dihydro-5H-pyrrolo[3,4-d]pyrimidin-4-yl]-quinolin-3-yl-amine;
[6-(2-Methoxy-benzyl)-6,7-dihydro-5H-pyrrolo[3,4-d]pyrimidin-4-yl]-quinolin-3-yl-amine;
[6-(2-Chloro-benzyl)-6,7-dihydro-5H-pyrrolo[3,4-d]pyrimidin-4-yl]-quinolin-3-yl-amine;
[6-(3-Methyl-furan-2-ylmethyl)-6,7-dihydro-5H-pyrrolo[3,4-d]pyrimidin-4-yl]-quinolin-3-yl-amine;
[6-(2,3-Dimethyl-benzyl)-6,7-dihydro-5H-pyrrolo[3,4-d]pyrimidin-4-yl]-quinolin-3-yl-amine;
[6-(2,5-Dimethyl-benzyl)-6,7-dihydro-5H-pyrrolo[3,4-d]pyrimidin-4-yl]-quinolin-3-yl-amine;
[6-(2-Ethyl-benzyl)-6,7-dihydro-5H-pyrrolo[3,4-d]pyrimidin-4-yl]-quinolin-3-yl-amine;
[6-(2-Methyl-benzyl)-6,7-dihydro-5H-pyrrolo[3,4-d]pyrimidin-4-yl]-quinolin-3-yl-amine;
(6-Pyridin-4-ylmethyl-6,7-dihydro-5H-pyrrolo[3,4-d]pyrimidin-4-yl)-quinolin-3-yl-amine;
Quinolin-3-yl-[6-(2-trifluoromethyl-benzyl)-6,7-dihydro-5H-pyrrolo[3,4-d]pyrimidin-4-yl]-amine;
[6-(2-Ethyl-4-methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzyl)-6,7-dihydro-5H-pyrrolo[3,4-d]pyrimidin-4-yl]-amine;
Quinolin-3-yl-[6-(3-trifluoromethyl-benzyl)-6,7-dihydro-5H-pyrrolo[3,4-d]pyrimidin-4-yl]-amine;
[6-(2-Ethoxy-benzyl)-6,7-dihydro-5H-pyrrolo[3,4-d]pyrimidin-4-yl]-quinolin-3-yl-amine;
[6-(2,3-Dihydro-benzofuran-7-ylmethyl)-6,7-dihydro-5H-pyrrolo[3,4-d]pyrimidin-4-yl]-quinolin-3-yl-amine;
[6-(2-Methylsulfanyl-benzyl)-6,7-dihydro-5H-pyrrolo[3,4-d]pyrimidin-4-yl]-quinolin-3-yl-amine;
(6-Benzo[1,3]dioxol-4-ylmethyl-6,7-dihydro-5H-pyrrolo[3,4-d]pyrimidin-4-yl)-quinolin-3-yl-amine;
Quinolin-3-yl-[6-(3-trifluoromethoxy-benzyl)-6,7-dihydro-5H-pyrrolo[3,4-d]pyrimidin-4-yl]-amine;
[6-(2-Difluoromethoxy-benzyl)-6,7-dihydro-5H-pyrrolo[3,4-d]pyrimidin-4-yl]-quinolin-3-yl-amine;
[6-(3-Methoxy-benzyl)-6,7-dihydro-5H-pyrrolo[3,4-d]pyrimidin-4-yl]-quinolin-3-yl-amine;
[6-(2,3-Difluoro-benzyl)-6,7-dihydro-5H-pyrrolo[3,4-d]pyrimidin-4-yl]-quinolin-3-yl-amine;
[6-(1H-Indol-7-ylmethyl)-6,7-dihydro-5H-pyrrolo[3,4-d]pyrimidin-4-yl]-quinolin-3-yl-amine;
[6-(3-Methyl-benzyl)-6,7-dihydro-5H-pyrrolo[3,4-d]pyrimidin-4-yl]-quinolin-3-yl-amine;
[6-(2-Cyclopropyl-benzyl)-6,7-dihydro-5H-pyrrolo[3,4-d]pyrimidin-4-yl]-quinolin-3-yl-amine; and
[6-(3-Methyl-butyl)-6,7-dihydro-5H-pyrrolo[3,4-d]pyrimidin-4-yl]-quinolin-3-yl-amine;

or a pharmaceutically acceptable salt, solvate or prodrug thereof;

and stereoisomers, isotopic variants and tautomers thereof.

Additional embodiments within the scope of the present invention are set forth in non-limiting fashion elsewhere herein and in the examples. It should be understood that these examples are for illustrative purposes only and are not to be construed as limiting this invention in any manner.

In certain aspects, the present invention provides prodrugs and derivatives of the compounds according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholinyl esters and the like.

Certain compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ or C₁-C₆alkyl, C₂-C₈ alkenyl, aryl, substituted aryl, and arylalkyl esters of the compounds of the invention.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound. In certain embodiments, the pharmaceutical composition may comprise a compound of the invention in combination with one or more compounds or compositions of like therapeutic utility and effect.

Generally, the compounds of this invention are administered in a pharmaceutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

The pharmaceutical compositions of this invention can be administered by a variety of routes including oral, rectal, transdermal, subcutaneous, intravenous, intramuscular, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the furansulfonic acid compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's The Science and Practice of Pharmacy, 21^{st} edition, 2005, Publisher: Lippincott Williams & Wilkins, which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1

### Tablets

A compound of the invention may be admixed as a dry powder with a dry gelatin binder in an approximate 1:2 weight ratio. A minor amount of magnesium stearate may be added as a lubricant. The mixture is formed into 240-270 mg tablets (80-90 mg of active compound per tablet) in a tablet press.

### Formulation 2

### Capsules

A compound of the invention may be admixed as a dry powder with a starch diluent in an approximate 1:1 weight ratio. The mixture is filled into 250 mg capsules (125 mg of active compound per capsule).

### Formulation 3

### Liquid

A compound of the invention (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then added to produce a total volume of 5 mL.

### Formulation 4

### Tablets

A compound of the invention may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

### Formulation 5

### Injection

A compound of the invention may be dissolved or suspended in a buffered sterile saline injectable aqueous medium to a concentration of approximately 5 mg/mL.

### Formulation 6

### Topical

Stearyl alcohol (250 g) and a white petrolatum (250 g) may be melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) may be added and the resulting mixture is stirred until it congeals.

### Methods of Treatment

The present compounds are used as therapeutic agents for the treatment of conditions in mammals. Accordingly, the compounds and pharmaceutical compositions of this invention find use as therapeutics for preventing and/or treating neurodegenerative, autoimmune and inflammatory conditions in mammals including humans. Thus, and as stated earlier, the present invention includes within its scope, and extends to, the recited methods of treatment, as well as to the compounds for use in such methods, and for the preparation of medicaments useful for such methods.

In a method of treatment aspect, this invention provides a method of treating a mammal susceptible to or afflicted with a condition associated with arthritis, asthma, dermatitis, myocardial infarction, inflammatory bowel disease and autoimmune disorders, which method comprises administering an effective amount of one or more of the pharmaceutical compositions just described.

In additional method of treatment aspects, this invention provides methods of treating a mammal susceptible to or afflicted with neurodegenerative diseases and disorders such as, for example Parkinson's disease, Alzheimer's disease and multiple sclerosis; sleep disorders, anxiety and depression disorders, weight and eating disorders, addiction, spasticity, and glaucoma; diseases and disorders which are mediated by or result in neuroinflammation such as, for example encephalitis; centrally-mediated neuropsychiatric diseases and disorders such as, for example depression mania, bipolar disease, anxiety, schizophrenia, eating disorders, sleep disorders and cognition disorders; epilepsy and seizure disorders; prostate, bladder and bowel dysfunction such as, for example urinary incontinence, urinary hesitancy, rectal hypersensitivity, fecal incontinence, benign prostatic hypertrophy and inflammatory bowel disease; respiratory and airway disease and disorders such as, for example, allergic rhinitis, asthma and reactive airway disease and chronic obstructive pulmonary disease; diseases and disorders which are mediated by or result in inflammation such as, for example rheumatoid arthritis and osteoarthritis, myocardial infarction, various autoimmune diseases and disorders; itch/pruritus such as, for example psoriasis; obesity; lipid disorders; nausea; emesis; cancer, and bone disorders; and renal disorders method comprises administering an effective condition-treating or condition-preventing amount of one or more of the pharmaceutical compositions just described.

In yet further method of treatment aspects, this invention provides a method of treating a mammal susceptible to or afflicted with a condition that gives rise to pain responses or that relates to imbalances in the maintenance of basal activity of sensory nerves. The present compounds have use as analgesics for the treatment of pain of various geneses or etiology, for example acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Bane syndrome, fibromyalgia, phantom limb pain, post-mastectomy pain, peripheral neuropathy, HIV neuropathy, and chemotherapy-induced and other iatrogenic neuropathies); visceral pain, (such as that associated with gastroesophageal reflex disease, irritable bowel syndrome, inflammatory bowel disease, pancreatitis, and various gynecological and urological disorders), dental pain and headache (such as migraine, cluster headache and tension headache).

As a further aspect of the invention there is provided the present compounds for use as a pharmaceutical especially in the treatment or prevention of the aforementioned conditions and diseases. We also provide the use of the present compounds in the manufacture of a medicament for the treatment or prevention of one of the aforementioned conditions and diseases.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as neurodegenerative and autoimmune conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound of the invention, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of a neurodegenerative, autoimmune or inflammatory condition, the compounds of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other active amines and derivatives.

### General Synthetic Procedures

The compounds of this invention can be prepared from readily available starting materials using the following general methods and procedures. See, e.g., Synthetic Scheme, below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds of this invention, for example, may be prepared by the reaction of a chloro derivative with an appropriately substituted amine and the product isolated and purified by known standard procedures. Such procedures include (but are not limited to) recrystallization, column chromatography or HPLC. The following schemes are presented with details as to the preparation of representative fused heterocyclics that have been listed hereinabove. The compounds of the invention may be prepared from known or commercially available starting materials and reagents by one skilled in the art of organic synthesis.

The compounds of the present invention may be prepared by a variety of processes well known for the preparation of compounds of this type, for example reaction schemes. and general procedures as described below.

The syntheses of representative compounds of this invention are carried out in accordance with the methods set forth above and using the appropriate reagents, starting materials, and purification methods known to those skilled in the art. All starting materials in the following general syntheses may be commercially available or obtained by conventional methods known to those skilled in the art.

In this specification, especially in “Representative Synthetic Methods”, the following abbreviations can be used:

BEP 2-bromo-1-ethylpyridinium tetrafluoroborate

BOP benzotriazol-1-yloxy-tris(dimethylamino)phosphonium hexafluorophosphate

CDI 2-chloro-1,3-dimethylimidazolinium chloride

DCC dicyclohexylcarbodiimide

DCM dichloromethane

DME 1,2-dimethoxyethane, dimethoxyethane

DMF N,N-dimethylformamide

DMSO dimethyl sulfoxide

EDC 1-ethyl-3-(3′-dimethylaminopropyl)carbodiimide hydrogen chloride

EtOAc ethyl acetate

EtOH ethanol

HOBt 1-hydroxybenzotriazole

MeOH methanol

NMP N-methyl-2-pyrrolidone

THF tetrahydrofuran

TFA trifluoroacetic acid

uM μM

uL μL

### Synthesis of Intermediates

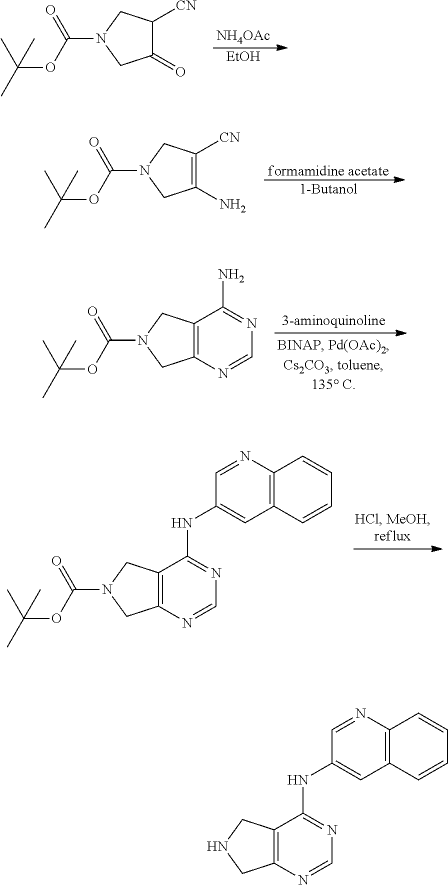

### Tert-butyl 3-amino-4-cyano-2,5-dihydro-1H-pyrrole-1-carboxylate

A mixture of tert-butyl 3-cyano-4-oxopyrrolidine-1-carboxylate (5.15 g, 24.5 mmol) and ammonium formate (2.32 g, 36.7 mmol) in ethanol (70 mL) was heated to reflux and stirred overnight. After allowing to cool to room temperature, the ethanol was removed under vacuum and the residue was partitioned between EtOAc (100 mL) and H₂O (100 mL). The aqueous layer was extracted with EtOAc (2×50 mL) and the combined organics were washed with brine (3×50 mL), dried (MgSO₄), filtered and concentrated under vacuum to leave a crude residue. The residue was purified by column chromatography on silica gel using EtOAc/hexanes (0 to 50% over 30 minutes) as eluent to give the product (3.09 g, 58%) as solid. ¹H NMR (400 MHz; d₆-DMSO) 6.84 (2H, d), 4.03-3.98 (4H, m), 1.40 (9H, s).

### Tert-butyl 4-amino-5H-pyrrolo[3,4-d]pyrimidine-6(7H)-carboxylate

Tert-butyl 3-amino-4-cyano-2,5-dihydro-1H-pyrrole-1-carboxylate (6.00 g, 28.7 mmol) and formamidine acetate (17.9 g, 172 mmol) were heated at reflux in 1-butanol (50 mL) for 3 days (after 3 days the reaction was not complete but was worked-up anyway). The solvent was removed under vacuum and the reaction mixture partitioned between EtOAc (200 mL) and water (200 mL). The aqueous layer was further extracted with EtOAc (3×50 mL) and the combined organics were washed with brine (3×100 mL), dried (MgSO₄), filtered and concentrated under vacuum to leave a crude residue. The residue was purified by column chromatography on silica gel using 0 to 5% MeOH in DCM over 70 minutes to give the desired product (3.08 g, 43%) as a solid. ¹H NMR (400 MHz; d₆-DMSO) 8.28 (s, 1H), 7.00 (s, 2H), 4.37 (s, 2H), 4.35 (s, 2H), 1.45 (d, J=5.2 Hz, 9H). In addition, to the desired product 1 g of unreacted starting material was recovered.

Alternatively, the reaction could be conducted using EtOH as solvent and heating under microwave irradiation (in a sealed vial) at 125° C. for about 2 hr.

### Tert-butyl 4-(quinolin-3-ylamino)-5H-pyrrolo[3,4-d]pyrimidine-6(7H)-carboxylate

In an open sealed tube, a mixture of palladium(II) acetate (71 mg, 0.32 mmol) and racemic-2,2′-bis(diphenylphosphino)-1,1′-binaphthyl (395 mg, 0.64 mmol) in toluene (15 mL) was stirred at room temperature for 10 min whilst being sparged with nitrogen. 3-Bromoquinoline (426 μL, 3.2 mmol) was then added and the mixture stirred at rt for 5 min then tert-butyl 4-amino-5H-pyrrolo[3,4-d]pyrimidine-6(7H)-carboxylate (0.75 g, 3.2 mmol) and Cs₂CO₃ (3.10 g, 9.5 mmol) added. The mixture was then sealed and heated to 135° C. and stirred overnight. After allowing to cool to room temperature (at this point TLC indicated complete reaction), the mixture was poured into EtOAc (100 mL) and H₂O (50 mL). The aqueous and organic layers were partitioned and the aqueous layer was extracted with EtOAc (50 mL). The combined organic extracts were washed with brine (1×30 mL), dried (MgSO₄), filtered and the solvent removed under vacuum to leave a crude residue. The residue was purified by column chromatography on silica gel (residue dry-loaded onto SiO₂) using 20-100% EtOAc/hexanes as eluent to give the product (0.75 g, 62%) as a solid. An analytical sample was triturated with EtOAc and filtered. The filter cake was washed with further EtOAc to give very pure product. ¹H NMR (400 MHz; d₆-DMSO) 9.57 (s, 1H), 9.14-9.13 (m, 1H), 8.86-8.83 (m, 1H), 8.66 (d, J=3.8 Hz, 1H), 7.97-7.92 (m, 2H), 7.66-7.56 (m, 2H), 4.66 (d, J=11.1 Hz, 2H), 4.52 (d, J=10.5 Hz, 2H), 1.51 (d, J=9.1 Hz, 9H).

### (6,7-Dihydro-5H-pyrrolo[3,4-d]pyrimidin-4-yl)-quinolin-3-yl-amine

Concentrated HCl (4.8 mL, 55 mmol) was added to a stirred solution of tert-butyl 4-(quinolin-3-yl-amino)-5H-pyrrolo[3,4-d]pyrimidine-6(7H)-carboxylate (4.0 g, 11.0 mmol) in methanol (100 mL) at room temperature. The mixture was heated to 65° C. and stirred for 2 hours. After cooling the product was filtered off as the dihydrochloride salt. The salt was dissolved in MeOH containing 2N HCl and was absorbed onto an SCX cartridge. The free base was eluted with 2N NH₃ in MeOH, and evaporated to give the desired product (2.2 g, 72%) as a solid. ¹H NMR (400 MHz; d₆-DMSO) 9.57 (1H, s), 9.13 (1H, d), 8.22 (1H, d), 8.64 (1H, s), 9.78-9.72 (2H, m), 7.66-7.56 (2H, m), 7.00 (1H, br. s), 4.36 (2H, s), 4.20 (2H, s).

### Representative Synthetic Methods

### Compound 1

### (6-Benzyl-6,7-dihydro-5H-pyrrolo[3,4-d]pyrimidin-4-yl)-quinolin-3-yl-amine

### Step 1

### 1-Benzyl-4-methoxymethyleneamino-2,5-dihydro-1H-pyrrole-3-carboxylic acid methyl ester (1B) and 6-Benzyl-3,5,6,7-tetrahydro-pyrrolo[3,4-d]pyrimidin-4-one (1C)

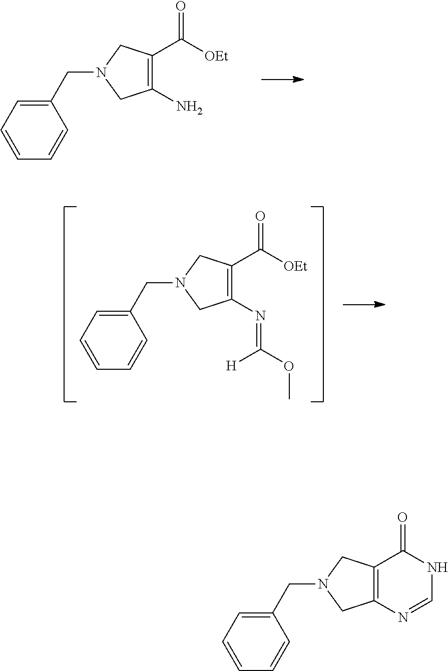

A mixture of the crude 4-amino-1-benzyl-2,5-dihydro-1H-pyrrole-3-carboxylic acid methyl ester (75.6 g, 0.325 mol) was treated with (250 mL, 2.3 mol) and acetic anhydride (250 mL, 2.6 mol), and the mixture heated to reflux for 3 h, then aged at room temperature overnight. The mixture was concentrated (bath temp=50° C.) to an orange oil (crude 99.6 g, 112%), which was carried forward to the next step. ¹H NMR (CDCl₃) showed evidence of the desired imino-ester in ˜20% purity (singlet @ 7.74 ppm).

2.0 M of Ammonia in ethanol (0.800 L, 1.60 mol) was added to the residue (assumed) 1-benzyl-4-methoxymethyleneamino-2,5-dihydro-1H-pyrrole-3-carboxylic acid methyl ester (99.6 g) and the warm mixture was stirred while ammonia gas (44 g, 2.6 mol, 8 equiv) was sparged through the stirred solution for 20 min. Aliquot after 1 h: LCMS showed significant evidence of product by both +Q and −Q ionization clear XIC for m/z=226.2/228.6 (M−H/M+H). Previous stability experiments (2 h; 424-DOM-063): 2D TLC in 10% MeOH/DCM, 2 M NaOH/DCM and 1 M H₃PO₄/DCM showed the pyrimidinone was stable to TLC and partially stable in 2 M NaOH. After 19 h, the mixture was concentrated to ˜150 mL, diluted with 2 M Na₂CO₃ (300 mL) and washed with DCM (2×200 mL). The aqueous layer was buffered to pH 6.5-7 with neat acetic acid (65 mL, 1.1 mol) and extracted with 3/1 CHCl₃/IPA (100 mL). The aqueous layer was saturated with sodium chloride, filtered through celite and extracted with 3/1 CHCl₃/IPA (3×100 mL). The combined organic extracts were dried (Na₂SO₄), filtered and concentrated to a light brown solid (13.2 g). Recrystallization from DCM/hexane afforded fine cream crystals (6.13 g, 8%). The mother liquors were concentrated and the residue was absorbed on silica (15 g). Chromatography on silica (Isco, 120 g cartridge, 0-10% MeOH/DCM) afforded the pyrimidinone (f11-32) as an off-white solid (3.42 g, 5%; net 9.55 g, 13%). ¹H NMR (400 MHz, CDCl₃) δ 13.21 (s, 1H), 8.06 (s, 1H), 7.39-7.25 (m, 5H), 3.98-3.91 (m, 4H), 3.91 (s, 2H); m/z=228.6 (M+H)+.

### Step 2

### 6-Benzyl-4-chloro-6,7-dihydro-5H-pyrrolo[3,4-d]pyrimidine

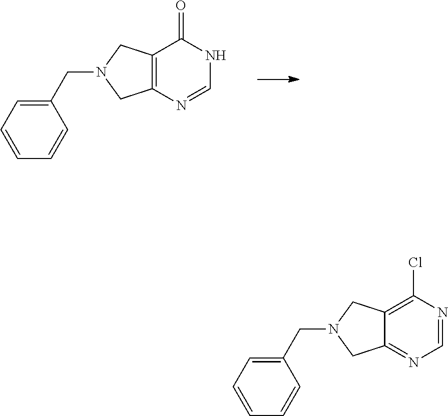

A 50 mL flask was charged with 6-benzyl-3,5,6,7-tetrahydro-pyrrolo[3,4-d]pyrimidin-4-one (135 mg, 0.594 mmol), N,N-dimethylaniline (0.15 mL, 1.2 mmol), N,N-dimethylformamide (2 uL, 0.02 mmol) and acetonitrile (7 mL, 100 mmol), and the mixture heated to reflux to dissolve the pyrimidinone. The mixture was allowed to cool, before neat phosphoryl chloride (0.22 mL, 2.4 mmol) was added dropwise (start 14:00) over 1 min. The light yellow suspension was then re-heated to reflux (14:05), and became clear after 15 min. Aliquot after 30 min: LCMS showed the reaction was complete. The mixture was removed from the heat after 40 min, and concentrated to an orange oil. The residue was taken up in EtOAc (20 mL), and quenched with ice (5 g), then partitioned between EtOAc (30 mL) and sat NaHCO₃ (50 mL). The aqueous layer was extracted with EtOAc (30 mL), and the combined organic layers were dried (Na₂SO₄), filtered and concentrated to a tan oil (256 mg), which was absorbed on silica (1 g). Column chromatography (Isco, 12 g cartridge, 0-3% MeOH/DCM over 40 min) afforded (f9-17) the chloropyrimidine (132 mg, 90%) as a pale yellow oil. ¹H NMR (400 MHz, CDCl₃) [d] 8.82 (s, 1H), 7.42-7.28 (m, 5H), 4.08 (s, 2H), 4.03 (s, 2H), 3.95 (s, 2H); m/z=246.4 (M+H)+.

### Step 3

### (6-Benzyl-6,7-dihydro-5H-pyrrolo[3,4-d]pyrimidin-4-yl)-quinolin-3-yl-amine

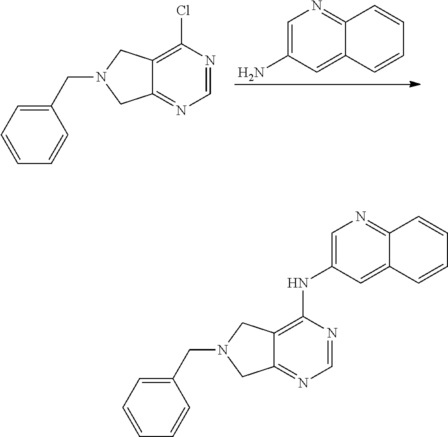

(6-Benzyl-6,7-dihydro-5H-pyrrolo[3,4-d]pyrimidin-4-yl)-quinolin-3-yl-amine (3C). A 250 mL flask was charged with [A] 3-Quinolinamine (3.51 g, 24.3 mmol), [B] 6-Benzyl-4-chloro-6,7-dihydro-5H-pyrrolo[3,4-d]pyrimidine (2.99 g, 12.2 mmol), Palladium Acetate (55 mg, 0.24 mmol), o-(Dicyclohexylphosphino)biphenyl (190 mg, 0.54 mmol) and Sodium tert-butoxide (1.87 g, 19.5 mmol). The flask was purged first with nitrogen, then with argon, whereupon argon-sparged Toluene (50 mL, 500 mmol) was added. The resulting red slurry was heated to 100 C in an oil bath (start 11:15). After 10 min, additional Toluene (40 mL) was added to improve stirrability. Aliquot after 22 h: LCMS showed the reaction was complete. The light orange slurry was diluted with water (100 mL) and EtOAc (100 mL) and extracted with 1 M H₃PO₄ (100 mL, 2×40 mL). The combined aqueous layers (pH=2) were basified to pH 4 with 6 M NaOH (25 mL), and the slurry filtered. The aqueous filtrate was basified to pH 10 with 6 M NaOH, and extracted with CHCl₃ (2×100 mL). The filter cake (primarily phosphate salt of the desired product) was stirred with a mixture of 1 M NaOH (200 mL) and DCM (200 mL), and the aqueous phase extracted with DCM (100 mL). The combined CHCl₃ and DCM extracts were dried (Na₂SO₄), filtered through celite and concentrated to an orange solid (4.98 g), which was absorbed on silica (13 g). Chromatography on silica (Isco, 120 g cartridge, 0-8% MeOH/DCM) afforded the amine (f24-42) as a light yellow granular solid (3.03 g, 70%). ¹H NMR (DMSO-d6) showed the desired product with trace contaminants. ¹H NMR (400 MHz, DMSO-d6) [d] 9.35 (s, 1H), 9.06 (d, J=2.5 Hz, 1H), 8.81 (d, J=2.5 Hz, 1H), 8.61 (s, 1H), 7.94 (d with fine str., J=8.3 Hz, 1H), 7.91 (dd, J=1.4, 8.0 Hz, 1H), 7.62 (ddd, J=1.4, 6.9, 8.3 Hz, 1H), 7.56 (ddd, J=1.3, 6.9, 8.0 Hz, 1H), 7.44 (d with fine str., J=8.4 Hz, 2H), 7.39 (app t, J=7.5 Hz, 2H), 7.30 (tt, J=1.4, 7.1 Hz, 1H), 3.95 (s, 2H), 3.94 (s, 2H), 3.91 (s, 2H); m/z=354.2 (M+H)+.

### Compound 2

### (6-Cyclohexylmethyl-6,7-dihydro-5H-pyrrolo[3,4-d]pyrimidin-4-yl)-quinolin-3-yl-amine

### Step 1

### (6,7-Dihydro-5H-pyrrolo[3,4-d]pyrimidin-4-yl)-quinolin-3-yl-amine

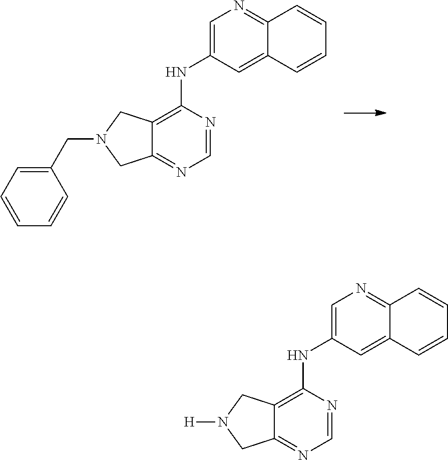

A 50 mL flask was charged with (6-benzyl-6,7-dihydro-5H-pyrrolo[3,4-d]pyrimidin-4-yl)-quinolin-3-yl-amine (60 mg, 0.17 mmol), ethanol (3 mL, 50 mmol) and acetic acid (0.05 mL, 0.9 mmol). Tetrahydrofuran (2 mL, 20 mmol) was added to the yellow suspension until the mixture clarified, followed by 20 wt % Palladium hydroxide on carbon (1:4, Palladium hydroxide:carbon black, 12 mg, 0.017 mmol). The reaction flask was evacuated and flushed with hydrogen 3 times and the mixture hydrogenated at room temperature overnight. Aliquot after 21 h: LCMS showed <20% conversion, with the same apparent mixture of 2 reduction products seen in 424-126. The mixture was heated in an oil bath at 60° C. Aliquot after 2.5 h; diluted with 5% MeCN in 1% aqueous HCO₂H: LCMS showed separation of the undesired 5,6,7,8-tetrahydroquinoline [THQ] (13%; tR=0.50 min) from desired amine (28%; tR=0.45 min) with 65% SM/1,2,3,4-THQ remaining. The mixture was filtered through celite, concentrated to a yellow oil (crude 82 mg), and the residue purified by reverse-phase HPLC (10-75% ACN in 10 mM Et₂NH/H₂O) to afford (f12) the secondary amine (8.5 mg, 19%) as a cream solid.

### Step 2

### (6-Cyclohexylmethyl-6,7-dihydro-5H-pyrrolo[3,4-d]pyrimidin-4-yl)-quinolin-3-yl-amine

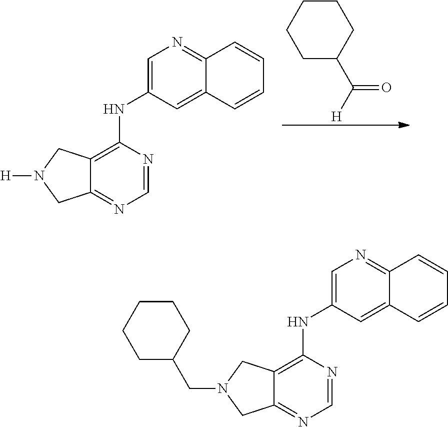

A 1.5 mL vial was charged with (6,7-dihydro-5H-pyrrolo[3,4-d]pyrimidin-4-yl)-quinolin-3-yl-amine (8.0 mg, 0.030 mmol), cyclohexanecarboxaldehyde (5.5 uL, 0.046 mmol), acetic acid (3.4 uL, 0.061 mmol) and dimethyl sulfoxide (300 uL, 4 mmol). Sodium triacetoxyborohydride (19 mg, 0.091 mmol) was added and the mixture was stirred at room temperature overnight (start 17:40). Aliquot after 30 min: LCMS showed the reaction was complete and very clean. Aliquot after 15 h: LCMS showed no formation of the bis-alkylated product, and the reaction is still quite clean. The mixture was purified by reverse-phase HPLC (25-80% ACN in 10 mM Et2NH/H₂O) to afford the amine as a cream solid (4.4 mg, 40%).

¹H NMR (400 MHz, DMSO-d6) [d] 9.36 (br s, 1H), 9.10 (d, J=2.5 Hz, 1H), 8.83 (d, J=2.5 Hz, 1H), 8.60 (s, 1H), 7.96 (d with fine str., J=8.3 Hz, 1H), 7.92 (d with fdine str., J=7.8 Hz, 1H), 7.61 (app t with fine str., J=7.9 Hz, 1H), 7.57 (app t with fine str., J=7.5 Hz, 1H), 3.95 (s, 2H), 3.83 (s, 2H), 2.56 (d, J=7.1 Hz, 2H), 1.83 (br d, J=13.6 Hz, 2H), 1.77-1.62 (m, 3H), 1.61-1.47 (m, 1H), 1.33-1.12 (m, 3H), 1.00-0.86 (m, 2H); m/z=360.4 (M+H)+.

### Compounds 3-19

### General Synthetic Method

### Library Synthesis

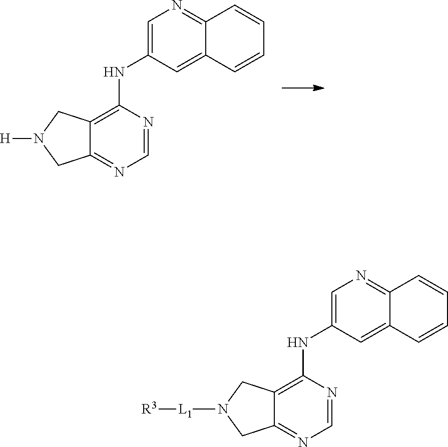

A 1.5 mL vial was charged with a solution of (6,7-dihydro-5H-pyrrolo[3,4-d]pyrimidin-4-yl)-quinolin-3-yl-amine (6.0 mg, 0.023 mmol) and the corresponding carboxaldehyde (0.046 mmol) in dimethyl sulfoxide (150 uL, 2.1 mmol). A solution of Sodium triacetoxyborohydride (14 mg, 0.068 mmol) and acetic acid (2.6 uL, 0.046 mmol) in dimethyl sulfoxide (150 uL, 2.1 mmol) was added and the mixture stirred at room temperature (start 14:00). Aliquot (3 uL) after 2 h: LCMS showed complete conversion. The mixtures were purified by reverse-phase HPLC (25-80% ACN in 10 mM Et₂NH/H₂O) to afford the desired pyrrolopyrimidine derivative.

### Compound 20

### 4-[4-(Quinolin-3-ylamino)-5,7-dihydro-pyrrolo[3,4-d]pyrimidin-6-yl]-butan-1-ol

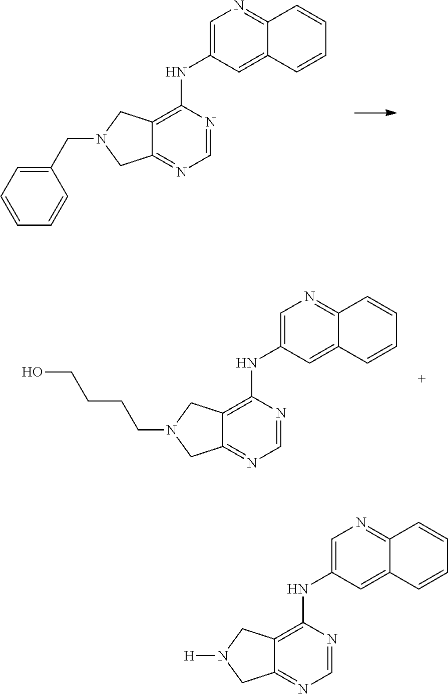

A 50 mL flask was charged with (6-benzyl-6,7-dihydro-5H-pyrrolo[3,4-d]pyrimidin-4-yl)-quinolin-3-yl-amine (60 mg, 0.17 mmol), ethanol (3 mL, 50 mmol), tetrahydrofuran (1.5 mL, 18 mmol) and 10% Palladium on Carbon (10:90, Palladium:carbon black, 9.0 mg, 0.0085 mmol), and the mixture was evacuated and flushed with hydrogen 3 times, then placed in an oil bath at 60° C. (start 19:40). Aliquot after 13.5 h: LCMS showed 30% SM consumption, 15% of the desired product, along with 16% of quinoline reduction, ie. a lower conversion efficiency than 20% Pd(OH)₂/C [424-127]. The crude material was filtered through celite, combined with material from 424-126, and purified by reverse-phase HPLC (10-75% ACN in 10 mM Et₂NH/H₂O) to afford partially purified samples (f16, 19, 22 & 25) of [C] (24 mg), incorrectly thought to be the tetrahydro-quinoline (m/z=358 for M+H is M+Na for the isolated alcohol). Further purification (10-25% ACN in 0.1% HCO₂H/H₂O; neutralization with MP-carbonate resin) afforded (f18 & 20) the purified tertiary amino-alcohol [C] (12.3 mg, 22%) as a white solid.

¹H NMR (400 MHz, DMSO-d6) δ 9.38 (br s, 1H), 9.10 (d, J=2.5 Hz, 1H), 8.82 (d, J=2.4 Hz, 1H), 8.60 (s, 1H), 7.96 (d with fine str., J=8.2 Hz, 1H), 7.92 (d with fine str., J=8.0 Hz, 1H), 7.63 (app dt, J=1.2, 7.0 Hz, 1H), 7.57 (app dt, J=1.3, 8.0 Hz, 1H), 3.97 (s, 2H), 3.84 (s, 2H), 3.45 (t, J=6.2 Hz, 2H), 2.73 (t, J=6.9 Hz, 2H2-1.47 (m, 4H); m/z=336.4 (M+H)+.

### Compound 24

### [6-(2-Methyl-2H-pyrazol-3-ylmethyl)-6,7-dihydro-5H-pyrrolo[3,4-d]pyrimidin-4-yl]-quinolin-3-yl-amine

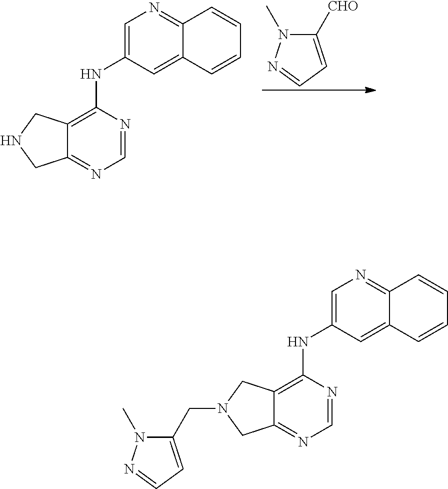

A 1.5 mL vial was charged with a solution of (6,7-dihydro-5H-pyrrolo[3,4-d]pyrimidin-4-yl)-quinolin-3-yl-amine (6.0 mg, 0.023 mmol) and 2-methyl-2H-pyrazole-3-carbaldehyde (0.046 mmol) in dimethyl sulfoxide (150 uL, 2.1 mmol). A solution of sodium triacetoxyborohydride (14 mg, 0.068 mmol) and acetic acid (2.6 uL, 0.046 mmol) in dimethyl sulfoxide (150 uL, 2.1 mmol) was added and the mixture stirred at room temperature. Aliquot (3 uL) after 2 h showed complete conversion. The mixtures were purified by reverse-phase HPLC (25-80% ACN in 10 mM Et₂NH/H₂O) to afford the title compound.

### Compound 28

### [6-(2,4-Dimethyl-thiazol-5-ylmethyl)-6,7-dihydro-5H-pyrrolo[3,4-d]pyrimidin-4-yl]-quinolin-3-yl-amine

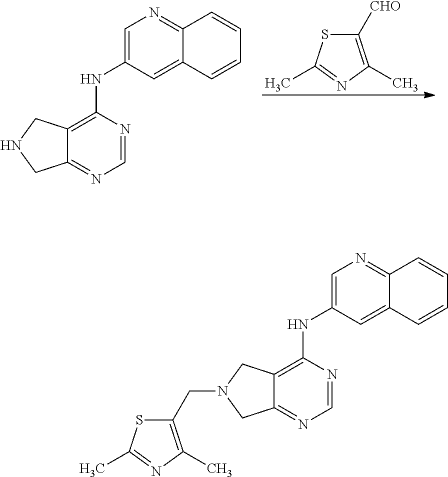

A vial was charged with (6,7-dihydro-5H-pyrrolo[3,4-d]pyrimidin-4-yl)-quinolin-3-yl-amine (100 mg, 0.4 mmol), 2,4-dimethylthiazole-5-carboxaldehyde (59 mg, 0.42 mmol), 1,2-dichloroethane (2 mL, 30 mmol) and methanol (0.4 mL, 10 mmol). Sodium triacetoxyborohydride (0.24 g, 1.1 mmol) was added, and the reaction stirred at room temperature for 2 hours. Saturated NaHCO3 solution was added, and the product extracted into DCM (3×3 mL). The combined organics were washed with brine (5 mL), dried (MgSO₄), filtered and concentrated. Purification by prep. HPLC, followed by flash chromatography (0 to 8% MeOH in DCM) gave [6-(2,4-dimethyl-thiazol-5-ylmethyl)-6,7-dihydro-5H-pyrrolo[3,4-d]pyrimidin-4-yl]-quinolin-3-yl-amine (29.2 mg) as a pale yellow solid.

### Compound 31

### Quinolin-3-yl-[6-(3,3,3-trifluoro-propyl)-6,7-dihydro-5H-pyrrolo[3,4-d]pyrimidin-4-yl]-amine

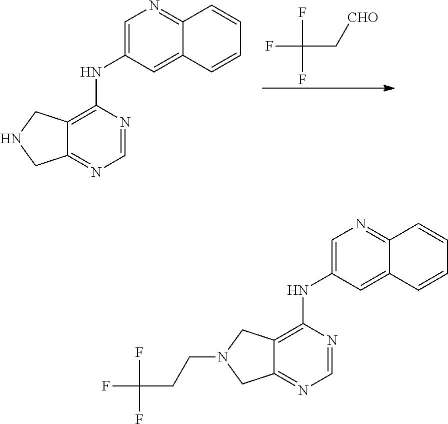

A 1.5 mL vial was charged with (6,7-dihydro-5H-pyrrolo[3,4-d]pyrimidin-4-yl)-quinolin-3-yl-amine (15.0 mg, 0.0570 mmol), 3,3,3-trifluoropropionaldehyde (9.6 mg, 0.085 mmol), acetic acid (6.5 uL, 0.11 mmol) and dimethyl sulfoxide (400 uL, 6 mmol). Sodium triacetoxyborohydride (36 mg, 0.17 mmol) was added and the mixture was stirred at room temperature overnight. Aliquot taken after 30 min showed ˜15% conversion by LCMS. Additional 3,3,3-trifluoropropionaldehyde (9.6 mg) was added after 40 min. Aliquot taken after 1 h showed ˜25% conversion. Additional 3,3,3-Trifluoropropionaldehyde (20 mg), sodium triacetoxyborohydride (36 mg) and acetic acid (6.5 uL) were added after 1.3 h. Aliquot taken after 3 h showed ˜60% conversion. The mixture was purified by reverse-phase HPLC (10-75% ACN in 0.1% HCO₂H/H₂O) to afford impure amine as a yellow solid (8.1 mg). Repurification by reverse-phase HPLC (25-80% ACN in 10 mM Et₂NH/H₂O) afforded quinolin-3-yl-[6-(3,3,3-trifluoro-propyl)-6,7-dihydro-5H-pyrrolo[3,4-d]pyrimidin-4-yl]-amine (3.8 mg, 18%) as a cream solid.

### Compound 33

### Quinolin-3-yl-[6-(tetrahydro-pyran-4-ylmethyl)-6,7-dihydro-5H-pyrrolo[3,4-d]pyrimidin-4-yl]-amine

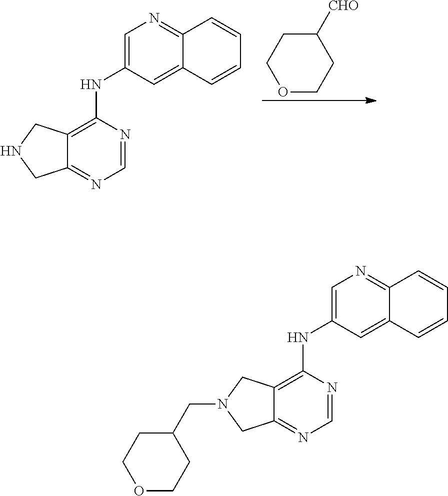

A 1.5 mL vial was charged with (6,7-dihydro-5H-pyrrolo[3,4-d]pyrimidin-4-yl)-quinolin-3-yl-amine (10 mg, 0.038 mmol), tetrahydro-2H-pyran-4-carbaldehyde (6.5 mg, 0.057 mmol), acetic acid (2.6 uL, 0.046 mmol) and dimethyl sulfoxide (300 uL, 4 mmol). Sodium triacetoxyborohydride (24 mg, 0.11 mmol) was added and the mixture was stirred at room temperature. Aliquot taken after 10 min showed the reaction was complete. After 2.5 h the mixture was purified by reverse-phase HPLC (25-80% ACN in 10 mM Et₂NH/H₂O) to afford quinolin-3-yl-[6-(tetrahydro-pyran-4-ylmethyl)-6,7-dihydro-5H-pyrrolo[3,4-d]pyrimidin-4-yl]-amine as a tan solid (9.4 mg, 67%).

### Compound 36

### [6-(1-Cyclobutyl-ethyl)-6,7-dihydro-5H-pyrrolo[3,4-d]pyrimidin-4-yl]-quinolin-3-yl-amine

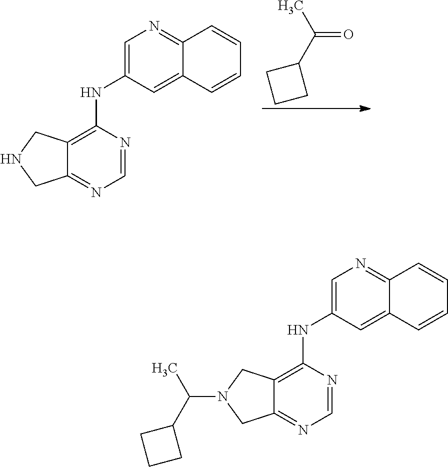

A 1.5 mL vial was charged with (6,7-dihydro-5H-pyrrolo[3,4-d]pyrimidin-4-yl)-quinolin-3-yl-amine (10 mg, 0.038 mmol) and 1-cyclobutyl-ethanone (0.049 mmol), whereupon a solution of sodium triacetoxyborohydride (24 mg, 0.11 mmol) and acetic acid (2.6 uL, 0.046 mmol) in dimethyl sulfoxide (300 uL, 4 mmol) was added and the mixture was stirred at room temperature. Aliquot taken after 2 h showed reaction was complete. After 3 h, the mixtures were filtered and purified by reverse-phase HPLC (10-45% ACN in 0.1% HCO₂H/H₂O; neutralized with MP-carbonate resin) to afford [6-(1-cyclobutyl-ethyl)-6,7-dihydro-5H-pyrrolo[3,4-d]pyrimidin-4-yl]-quinolin-3-yl-amine as a solid.

### Compound 37

### [6-(1-Cyclopentyl-ethyl)-6,7-dihydro-5H-pyrrolo[3,4-d]pyrimidin-4-yl]-quinolin-3-yl-amine

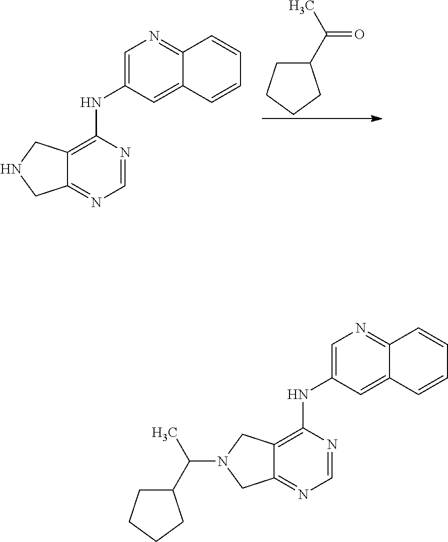

A 1.5 mL vial was charged with (6,7-dihydro-5H-pyrrolo[3,4-d]pyrimidin-4-yl)-quinolin-3-yl-amine (10 mg, 0.038 mmol) and 1-cyclopentyl-ethanone (0.049 mmol), whereupon a solution of sodium triacetoxyborohydride (24 mg, 0.11 mmol) and acetic acid (2.6 uL, 0.046 mmol) in dimethyl sulfoxide (300 uL, 4 mmol) was added, and the mixture was stirred at room temperature. Aliquot taken after 2 h showed reaction was complete. After 3 h, the mixtures were filtered and purified by reverse-phase HPLC (10-45% ACN in 0.1% HCO2H/H2O; neutralized with MP-carbonate resin) to afford [6-(1-cyclopentyl-ethyl)-6,7-dihydro-5H-pyrrolo[3,4-d]pyrimidin-4-yl]-quinolin-3-yl-amine as a solid.

### Compound 38

### [6-(1-Cyclohexyl-ethyl)-6,7-dihydro-5H-pyrrolo[3,4-d]pyrimidin-4-yl]-quinolin-3-yl-amine

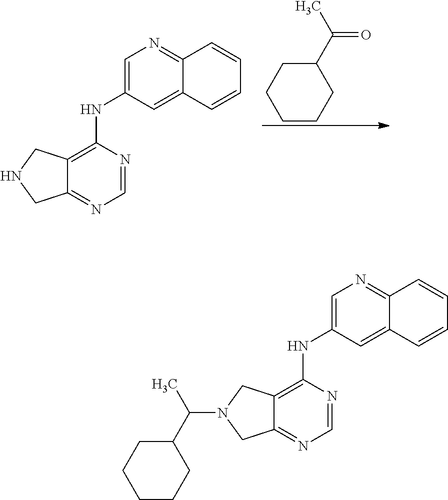

A 1.5 mL vial was charged with (6,7-dihydro-5H-pyrrolo[3,4-d]pyrimidin-4-yl)-quinolin-3-yl-amine (10 mg, 0.038 mmol) and 1-cyclohexyl-ethanone (0.049 mmol), whereupon a solution of sodium triacetoxyborohydride (24 mg, 0.11 mmol) and acetic acid (2.6 uL, 0.046 mmol) in dimethyl sulfoxide (300 uL, 4 mmol) were added and the mixture was stirred at room temperature. Aliquot taken after 2 h showed reaction was complete. After 3 h, the mixtures were filtered and purified by reverse-phase HPLC (10-45% ACN in 0.1% HCO₂H/H₂O; neutralized with MP-carbonate resin) to afford the tertiary amine as a solid.

### Compound 40

### (6-Cycloheptyl-6,7-dihydro-5H-pyrrolo[3,4-d]pyrimidin-4-yl)-quinolin-3-yl-amine

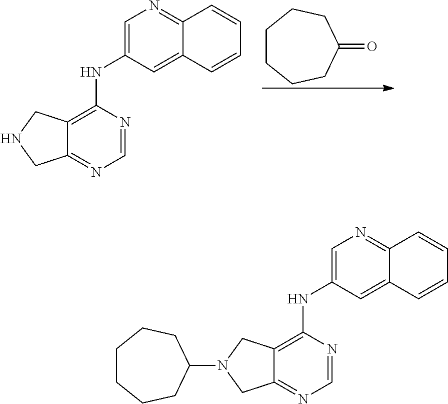

A 1.5 mL vial was charged with (6,7-dihydro-5H-pyrrolo[3,4-d]pyrimidin-4-yl)-quinolin-3-yl-amine (10 mg, 0.038 mmol) and cycloheptanone (0.049 mmol), whereupon a solution of sodium triacetoxyborohydride (24 mg, 0.11 mmol) and acetic acid (2.6 uL, 0.046 mmol) in dimethyl sulfoxide (300 uL, 4 mmol) was added, and the mixture was stirred at room temperature. Aliquot taken after 2 h showed reactions complete. After 3 h, the mixtures were filtered and purified by reverse-phase HPLC (10-45% ACN in 0.1% HCO₂H/H₂O; neutralized with MP-carbonate resin) to afford (6-cycloheptyl-6,7-dihydro-5H-pyrrolo[3,4-d]pyrimidin-4-yl)-quinolin-3-yl-amine as a solid.

### Compound 41

### (6-Cyclopentyl-6,7-dihydro-5H-pyrrolo[3,4-d]pyrimidin-4-yl)-quinolin-3-yl-amine

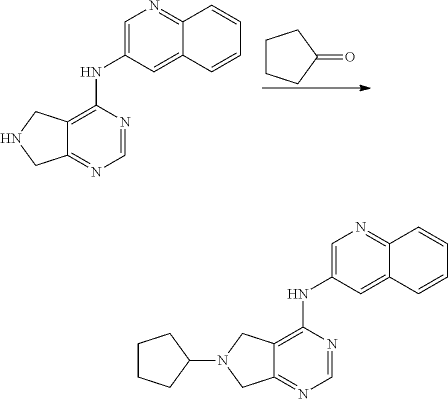

A 1.5 mL vial was charged with (6,7-dihydro-5H-pyrrolo[3,4-d]pyrimidin-4-yl)-quinolin-3-yl-amine (10 mg, 0.038 mmol) and cyclopentanone (0.049 mmol), whereupon a solution of sodium triacetoxyborohydride (24 mg, 0.11 mmol) and acetic acid (2.6 uL, 0.046 mmol) in dimethyl sulfoxide (300 uL, 4 mmol) was added (start 11:30), and the mixture was stirred at room temperature. Aliquot taken after 2 h showed reaction was complete. After 3 h, the mixtures were filtered and purified by reverse-phase HPLC (10-45% ACN in 0.1% HCO₂H/H₂O; neutralized with MP-carbonate resin) to afford (6-Cyclopentyl-6,7-dihydro-5H-pyrrolo[3,4-d]pyrimidin-4-yl)-quinolin-3-yl-amine as a solid.

### Compound 45

### 4-(Quinolin-3-ylamino)-5,7-dihydro-pyrrolo[3,4-d]pyrimidine-6-carboxylic acid tert-butyl ester

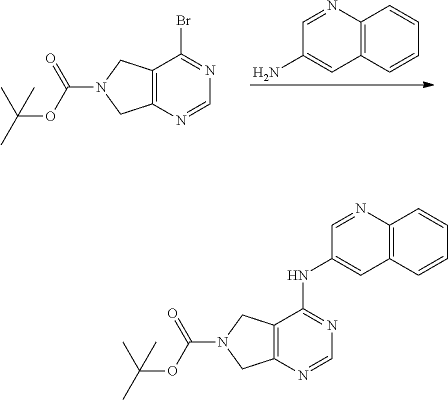

In an open sealed tube, a mixture of palladium acetate (71.3 mg, 0.317 mmol) (R)-(+)-2,2′-bis(diphenylphosphino)-1,1′-binaphthyl (395 mg, 0.635 mmol) in toluene (15 mL, 140 mmol) was stirred at room temperature for 10 min whilst being splurged with nitrogen. Quinoline, 3-bromo- (426 uL, 3.17 mmol) was then added and the mixture stirred at rt for 5 min then tert-butyl 4-amino-5H-pyrrolo[3,4-d]pyrimidine-6(7H)-carboxylate (0.750 g, 3.17 mmol) and cesium carbonate (3.10 g, 9.52 mmol) added. The mixture was then sealed and heated to 135° C. and stirred overnight. After allowing to cool to room temperature (at this point TLC indicated complete reaction), the mixture was poured into EtOAc (100 mL) and H₂O (50 mL). The aqueous and organic layers were partitioned and the aqueous layer was extracted with EtOAc (50 mL). The combined organic extracts were washed with brine (1×30 mL), dried (MgSO₄), filtered and the solvent removed to leave a crude residue. The residue was purified by column chromatography on silica gel (residue dry-loaded onto SiO₂) using 20-100% EtOAc/hexanes as eluent to give 4-(quinolin-3-ylamino)-5,7-dihydro-pyrrolo[3,4-d]pyrimidine-6-carboxylic acid tert-butyl (0.75 g) as a solid.

### Compound 46

### 4-(Quinolin-3-ylamino)-5,7-dihydro-pyrrolo[3,4-d]pyrimidine-6-carboxylic acid cyclohexylmethyl-amide

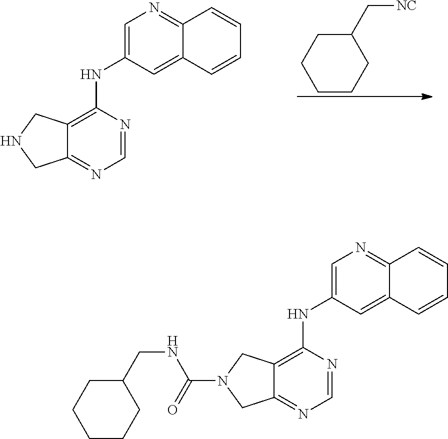

A solution of isocyanatomethyl-cyclohexane (7.66 mg, 0.0550 mmol) in toluene (130 uL, 1.2 mmol) was added to a solution of (6,7-dihydro-5H-pyrrolo[3,4-d]pyrimidin-4-yl)-quinolin-3-yl-amine (13 mg, 0.050 mmol) in N-methylpyrrolidinone (300 uL, 3 mmol), and the mixture stirred at room temperature. Aliquot taken after 45 min showed the reaction was complete. Purification by reverse-phase HPLC (25-60% ACN in 10 mM Et₂NH/H₂O) afforded 4-(quinolin-3-ylamino)-5,7-dihydro-pyrrolo[3,4-d]pyrimidine-6-carboxylic acid cyclohexylmethyl-amide as a cream solid (9.6 mg, 48%).

### Compound 47

### 4-(Quinolin-3-ylamino)-5,7-dihydro-pyrrolo[3,4-d]pyrimidine-6-carboxylic acid cyclohexylamide

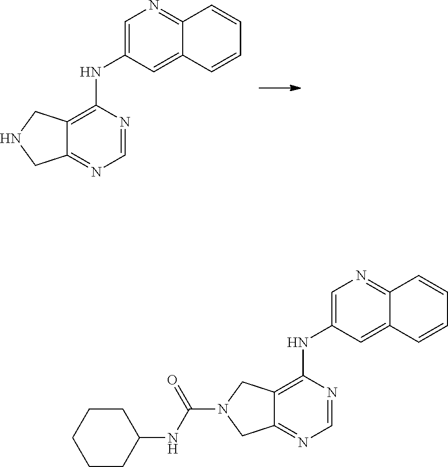

Cyclohexylisocyanate (7.0 uL, 0.055 mmol) was added to a solution of (6,7-dihydro-5H-pyrrolo[3,4-d]pyrimidin-4-yl)-quinolin-3-yl-amine (13 mg, 0.050 mmol) in N-methylpyrrolidinone (400 uL, 4 mmol), and the mixture stirred at room temperature. Aliquot taken after 45 min showed the reaction was complete. Purification by reverse-phase HPLC (25-60% ACN in 10 mM Et₂NH/H₂O) afforded 4-(quinolin-3-ylamino)-5,7-dihydro-pyrrolo[3,4-d]pyrimidine-6-carboxylic acid cyclohexylamide as a cream solid (10.1 mg, 52%).

### Compound 49

### (2,4-Dimethyl-thiazol-5-yl)-[4-(quinolin-3-ylamino)-5,7-dihydro-pyrrolo[3,4-d]pyrimidin-6-yl]-methanone

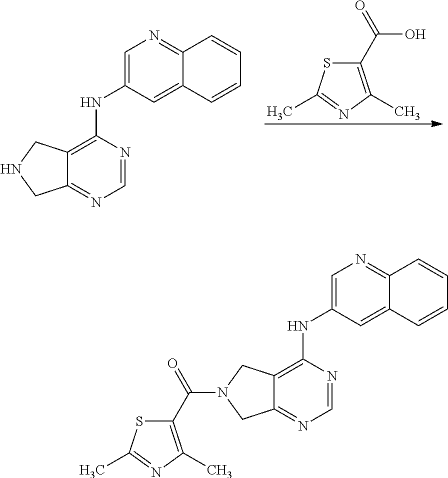

N,N-Diisopropylethylamine (35 uL, 0.20 mmol) was added to a suspension of (6,7-dihydro-5H-pyrrolo[3,4-d]pyrimidin-4-yl)-quinolin-3-yl-amine dihydrochloride (17 mg, 0.050 mmol), 2,4-dimethyl-thiazole-5-carboxylic acid (9.4 mg, 0.060 mmol), and N,N,N′,N′-tetramethyl-O-(7-azabenzotriazol-1-yl)uronium hexafluorophosphate (28 mg, 0.075 mmol) in N-methylpyrrolidinone (400 uL, 4 mmol) in a 1.5 mL vial, and the mixture was stirred at room temperature. Aliquot taken after 5 min showed the reaction was complete. Purification by reverse-phase HPLC (25-60% ACN in 10 mM Et₂NH/H₂O) afforded (2,4-dimethyl-thiazol-5-yl)-[4-(quinolin-3-ylamino)-5,7-dihydro-pyrrolo[3,4-d]pyrimidin-6-yl]-methanone as a cream solid (12.8 mg, 64%).

### Compound 53

### 2-Piperidin-1-yl-1-[4-(quinolin-3-ylamino)-5,7-dihydro-pyrrolo[3,4-d]pyrimidin-6-yl]-ethanone

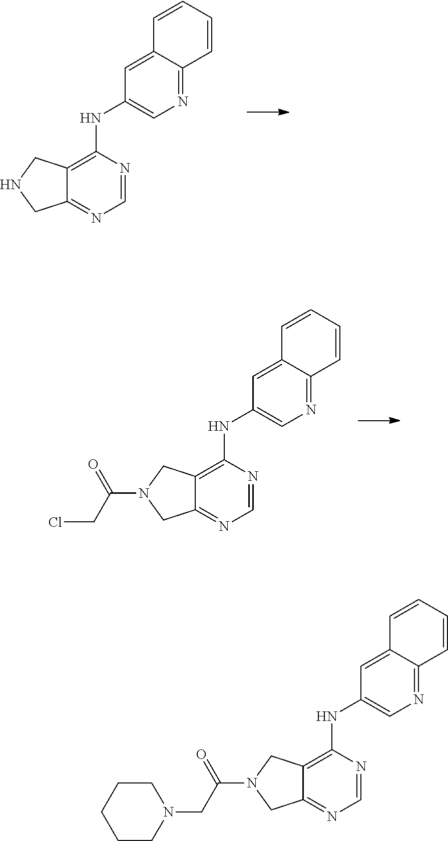

A solution of (6,7-dihydro-5H-pyrrolo[3,4-d]pyrimidin-4-yl)-quinolin-3-yl-amine (50 mg, 0.19 mmol) and pyridine (30 uL, 0.38 mmol) in chloroform (5 mL, 60 mmol) and methanol (0.1 mL, 2 mmol) was treated with a solution of chloroacetyl chloride (15 uL, 0.19 mmol) in chloroform (0.5 mL), and the mixture stirred at room temperature (start 14:25). Aliquot taken after 1 min showed only 25% conversion. Additional methanol (0.2 mL), pyridine (75 uL, 5 equiv) and chloroacetyl chloride (75 uL, 5 equiv) in chloroform (1 mL) were added sequentially to the cloudy suspension after 15 min. Aliquot taken after an additional 2 min showed minimal extra conversion. The mixture was concentrated to a brown oil. Attempted dissolution in THF (5 mL) was unsuccessful. The mixture was dissolved in 3/1 CHCl₃/IPA (10 mL) and pyridine (150 uL, 10 equiv), whereupon a solution of chloroacetyl chloride (15 uL, 1.0 equiv) in chloroform (0.6 mL) was added dropwise over 30 s. Aliquot taken after 2 min showed ˜40% conversion. Additional chloroacetyl chloride (60 uL, 4 equiv) in CHCl₃ (0.5 mL) was added after 10 min. Aliquot taken after 10 min later showed ˜75% conversion. After 30 min, the mixture was poured into sat NaHCO₃ (20 mL) and CHCl₃ (20 mL). The aqueous layer was extracted with CHCl₃ (20 mL), and the combined organic layers were dried (Na₂SO₄), filtered and concentrated to a brown semi-solid (crude 75 mg), which was absorbed on silica (0.3 g). Chromatography on silica gel (Isco, 4 g cartridge, 0-8% MeOH/DCM) afforded the intermediate chloromethyl amide (f24-30) as a cream solid (19 mg, 29%).

¹H NMR (400 MHz, DMSO-d6) δ rotamers (9.68 (s, 1H, major), 9.63 (s, 1H, minor), 9.14 (d, J=2.5 Hz, 1H, major), 9.12 (d, J=2.5 Hz, 1H, minor), 8.88 (d, J=2.4 Hz, 1H, major), 8.84 (d, J=2.4 Hz, 1H, minor), 8.70 (s, 1H), 7.96 (app t, J=9.0 Hz, 2H), 7.68-7.62 (m, 1H), 7.61-7.55 (m, 1H), 4.96 (s, 2H, minor), 4.84 (s, 2H, major), 4.75 (s, 2H, major), 4.61 (s, 2H, minor), 4.53 (s, 2H, major), 4.49 (s, 2H, minor); m/z=340.2 (M+H)+.

### Step 2

A 1.5 mL vial was charged with 2-chloro-1-[4-(quinolin-3-ylamino)-5,7-dihydro-pyrrolo[3,4-d]pyrimidin-6-yl]-ethanone (6.0 mg, 0.018 mmol), piperidine (0.18 mmol) and dimethyl sulfoxide (0.4 mL, 6 mmol), and the mixture was heated in an oil bath at 80° C. overnight. Aliquot taken after 17.3 h showed the reactions was complete. Purification by reverse-phase HPLC (20-65% ACN in 10 mM Et₂NH/H₂O) afforded 2-piperidin-1-yl-1-[4-(quinolin-3-ylamino)-5,7-dihydro-pyrrolo[3,4-d]pyrimidin-6-yl]-ethanone as a solid.

### Compound 55

### [6-(5-Oxazol-5-yl-thiophene-2-sulfonyl)-6,7-dihydro-5H-pyrrolo[3,4-d]pyrimidin-4-yl]-quinolin-3-yl-amine

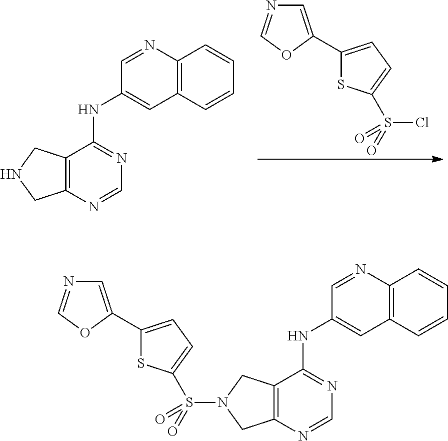

A 2 mL vial was charged with (6,7-dihydro-5H-pyrrolo[3,4-d]pyrimidin-4-yl)-quinolin-3-yl-amine dihydrochloride (12.000 mg, 0.035691 mmol), 5-oxazol-5-yl-thiophene-2-sulfonyl chloride (13.37 mg, 0.05354 mmol), N,N-diisopropylethylamine (31.08 uL, 0.1784 mmol) and N,N-dimethylformamide (800 uL, 10 mmol). The reaction was stirred overnight.

The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving [6-(5-oxazol-5-yl-thiophene-2-sulfonyl)-6,7-dihydro-5H-pyrrolo[3,4-d]pyrimidin-4-yl]-quinolin-3-yl-amine (3 mg) as a white solid. Purity was found to be 99.5% by analytical HPLC.

### Compound 56

### [6-(3-Methyl-thiophene-2-sulfonyl)-6,7-dihydro-5H-pyrrolo[3,4-d]pyrimidin-4-yl]-quinolin-3-yl-amine

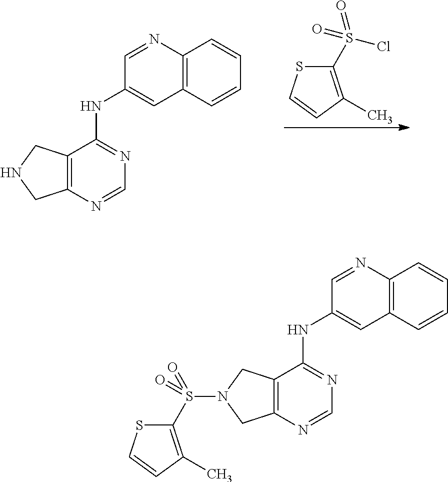

A 2 mL vial was charged with (6,7-dihydro-5H-pyrrolo[3,4-d]pyrimidin-4-yl)-quinolin-3-yl-amine dihydrochloride (12.00 mg, 0.035691 mmol), 3-methyl-thiophene-2-sulfonyl chloride (10.53 mg, 0.05354 mmol), N,N-diisopropylethylamine (31.08 uL, 0.1784 mmol) and N,N-dimethylformamide (800 uL, 10 mmol). The reaction was stirred overnight. The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving [6-(3-methyl-thiophene-2-sulfonyl)-6,7-dihydro-5H-pyrrolo[3,4-d]pyrimidin-4-yl]-quinolin-3-yl-amine (3 mg) as a white solid. Purity was found to be 98.7% by analytical HPLC

### Compound 58

### [6-(2,4-Difluoro-benzenesulfonyl)-6,7-dihydro-5H-pyrrolo[3,4-d]pyrimidin-4-yl]-quinolin-3-yl-amine

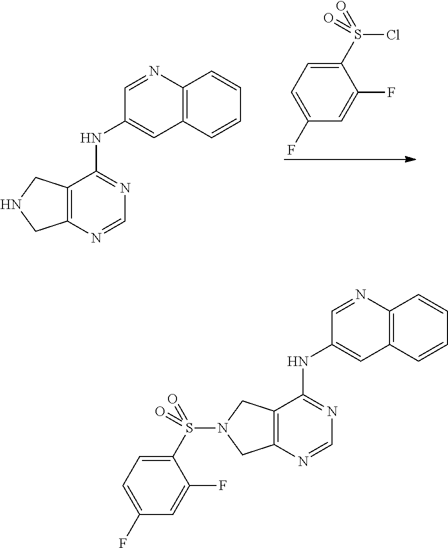

A 2 mL vial was charged with (6,7-dihydro-5H-pyrrolo[3,4-d]pyrimidin-4-yl)-quinolin-3-yl-amine dihydrochloride (12.000 mg, 0.035691 mmol), 2,4-difluoro-benzenesulfonyl chloride (11.38 mg, 0.05354 mmol), N,N-diisopropylethylamine (31.08 uL, 0.1784 mmol) and N,N-dimethylformamide (800 uL, 10 mmol). The reaction was stirred overnight. The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving [6-(2,4-difluoro-benzenesulfonyl)-6,7-dihydro-5H-pyrrolo[3,4-d]pyrimidin-4-yl]-quinolin-3-yl-amine (5 mg) as a white solid. Purity was 98.7% by analytical HPLC.

### Compound 59

### Quinolin-3-yl-[6-(toluene-2-sulfonyl)-6,7-dihydro-5H-pyrrolo[3,4-d]pyrimidin-4-yl]-amine

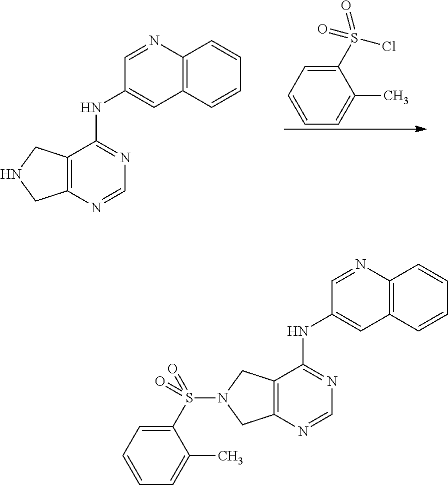

A 2 mL vial was charged with (6,7-dihydro-5H-pyrrolo[3,4-d]pyrimidin-4-yl)-quinolin-3-yl-amine dihydrochloride (12.000 mg, 0.035691 mmol), 2-methyl-benzenesulfonyl chloride (10.21 mg, 0.05354 mmol), N,N-diisopropylethylamine (31.08 uL, 0.1784 mmol) and N,N-dimethylformamide (800 uL, 10 mmol). The reaction was stirred overnight. The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving quinolin-3-yl-[6-(toluene-2-sulfonyl)-6,7-dihydro-5H-pyrrolo[3,4-d]pyrimidin-4-yl]-amine (3 mg) as a white solid. Purity was found to be 97.9% by analytical HPLC.

### Compound 60

### [6-(2-Methyl-2H-pyrazole-3-sulfonyl)-6,7-dihydro-5H-pyrrolo[3,4-d]pyrimidin-4-yl]-quinolin-3-yl-amine

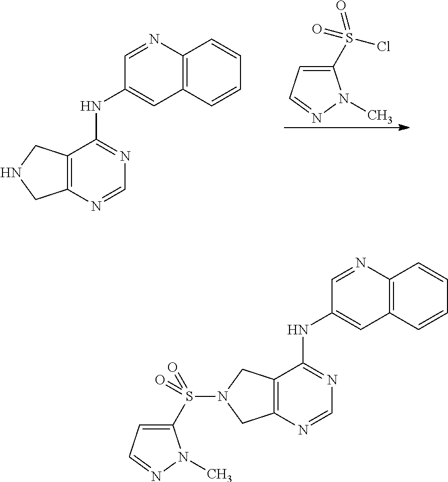

A 2 mL vial was charged with (6,7-dihydro-5H-pyrrolo[3,4-d]pyrimidin-4-yl)-quinolin-3-yl-amine dihydrochloride (12.000 mg, 0.035691 mmol), 1-methyl-1H-pyrazole-5-sulfonyl chloride (9.669 mg, 0.05354 mmol), N,N-diisopropylethylamine (31.08 uL, 0.1784 mmol) and N,N-dimethylformamide (800 uL, 10 mmol). The reaction was stirred overnight.

The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving 476-22-1 (4 mg) as a white solid. Purity was 97.0% by analytical HPLC.

### Compound 61

### [6-(2,4-Dimethyl-thiazole-5-sulfonyl)-6,7-dihydro-5H-pyrrolo[3,4-d]pyrimidin-4-yl]-quinolin-3-yl-amine

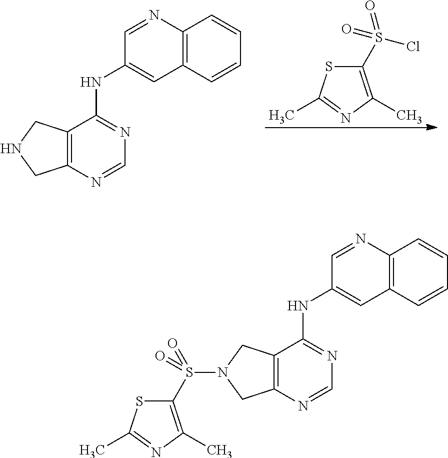

A 2 mL vial was charged with (6,7-dihydro-5H-pyrrolo[3,4-d]pyrimidin-4-yl)-quinolin-3-yl-amine dihydrochloride (12.00 mg, 0.035691 mmol), 2,4-dimethyl-thiazole-5-sulfonyl chloride (11.33 mg, 0.05354 mmol), N,N-diisopropylethylamine (31.08 uL, 0.1784 mmol) and N,N-dimethylformamide (800 uL, 10 mmol). The reaction was stirred overnight. The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving [6-(2,4-dimethyl-thiazole-5-sulfonyl)-6,7-dihydro-5H-pyrrolo[3,4-d]pyrimidin-4-yl]-quinolin-3-yl-amine (7 mg) as a white solid. Purity was 97.6% by analytical HPLC.

### Compound 64

### (6-Cyclohexanesulfonyl-6,7-dihydro-5H-pyrrolo[3,4-d]pyrimidin-4-yl)-quinolin-3-yl-amine

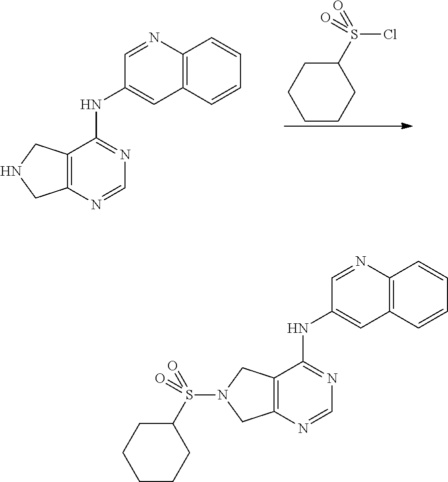

A 2 mL vial was charged with (6,7-dihydro-5H-pyrrolo[3,4-d]pyrimidin-4-yl)-quinolin-3-yl-amine dihydrochloride (12.000 mg, 0.035691 mmol), cyclohexanesulfonyl chloride (9.779 mg, 0.05354 mmol), N,N-diisopropylethylamine (31.08 uL, 0.1784 mmol) and N,N-dimethylformamide (800 uL, 10 mmol). The reaction was stirred overnight. The reaction mixture was quenched with N-ethylethanamine (18.46 uL, 0.1784 mmol). After filtering, the reaction mixture was injected directly onto a reversed-phase HPLC (acetonitrile-water at pH10) and the purified product was isolated, giving 476-17-1 (2.5 mg) as a white solid. Purity was 98.2% by analytical HPLC

### Compound 65

### (6-Benzenesulfonyl-6,7-dihydro-5H-pyrrolo[3,4-d]pyrimidin-4-yl)-quinolin-3-yl-amine

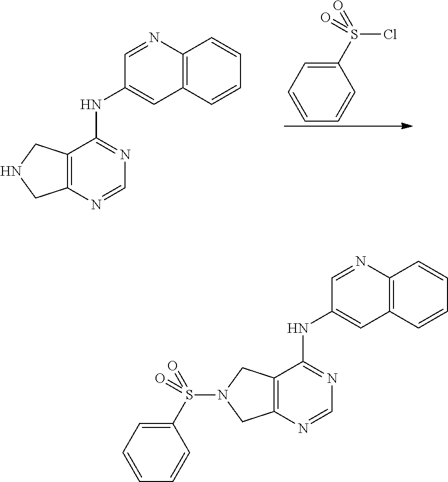

A 2 mL vial was charged with (6,7-dihydro-5H-pyrrolo[3,4-d]pyrimidin-4-yl)-quinolin-3-yl-amine dihydrochloride (12.000 mg, 0.035691 mmol), benzenesulfonyl chloride (9.456 mg, 0.05354 mmol), N,N-diisopropylethylamine (31.08 uL, 0.1784 mmol) and Acetonitrile (500 uL, 10 mmol). The reaction was stirred overnight. The reaction mixture was quenched with saturated NaHCO₃ (5 mL) and extracted into EtOAc (5 mL). The organic layer was evaporated to dryness. The crude material was purified by reversed-phase HPLC (acetonitrile-water at pH10) and the purified product was isolated, giving (6-benzenesulfonyl-6,7-dihydro-5H-pyrrolo[3,4-d]pyrimidin-4-yl)-quinolin-3-yl-amine (3 mg) as a white solid. Purity was 100% by analytical HPLC.

### Compound 66

### [6-(4-Methyl-thiazol-5-ylmethyl)-6,7-dihydro-5H-pyrrolo[3,4-d]pyrimidin-4-yl]-quinolin-3-yl-amine

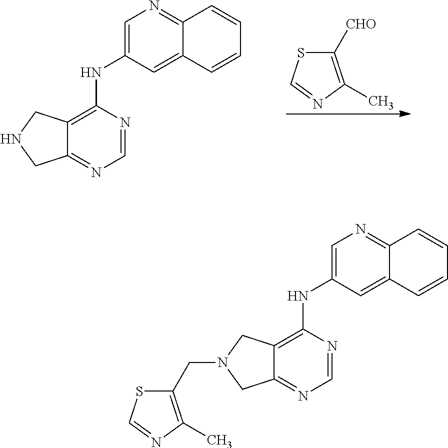

A vial was charged with (6,7-dihydro-5H-pyrrolo[3,4-d]pyrimidin-4-yl)-quinolin-3-yl-amine (10.0 mg, 0.0380 mmol), 4-methyl-thiazole-5-carbaldehyde (5.31 mg, 0.0418 mmol), 1,2-dichloroethane (0.5 mL, 6 mmol) and methanol (0.1 mL, 2 mmol). Sodium triacetoxyborohydride (24.1 mg, 0.114 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(4-methyl-thiazol-5-ylmethyl)-6,7-dihydro-5H-pyrrolo[3,4-d]pyrimidin-4-yl]-quinolin-3-yl-amine (2.4 mg) as an off-white solid.

### Compound 68

### 5-[4-(Quinolin-3-ylamino)-5,7-dihydro-pyrrolo[3,4-d]pyrimidine-6-sulfonyl]-furan-2-carboxylic acid methyl ester

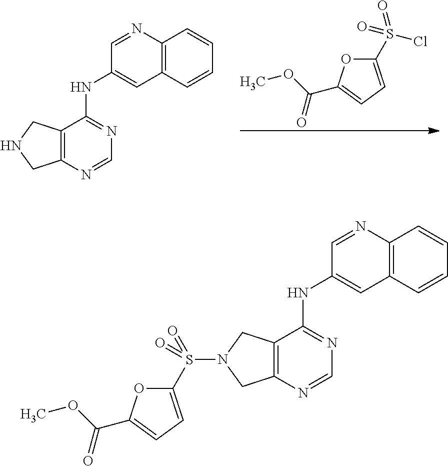

A vial was charged with (6,7-dihydro-5H-pyrrolo[3,4-d]pyrimidin-4-yl)-quinolin-3-yl-amine dihydrochloride (12.000 mg, 0.035691 mmol), 5-chlorosulfonyl-furan-2-carboxylic acid methyl ester (12.02 mg, 0.05354 mmol), N,N-diisopropylethylamine (31.08 uL, 0.1784 mmol) and acetonitrile (500 uL, 10 mmol). The reaction was stirred for 1 hour at room temperature. Saturated NaHCO₃ solution (5 mL) was added, and the mixture extracted with EtOAc (3×5 mL). The combined organics were washed with brine (3×5 mL), dried (MgSO₄), filtered and concentrated. Flash chromatography (0 to 5% MeOH in DCM over 45 minutes) gave 5-[4-(quinolin-3-ylamino)-5,7-dihydro-pyrrolo[3,4-d]pyrimidine-6-sulfonyl]-furan-2-carboxylic acid methyl ester (4 mg) as a pale yellow solid.

### Compound 69

### [6-(2-Methyl-thiazol-5-ylmethyl)-6,7-dihydro-5H-pyrrolo[3,4-d]pyrimidin-4-yl]-quinolin-3-yl-amine

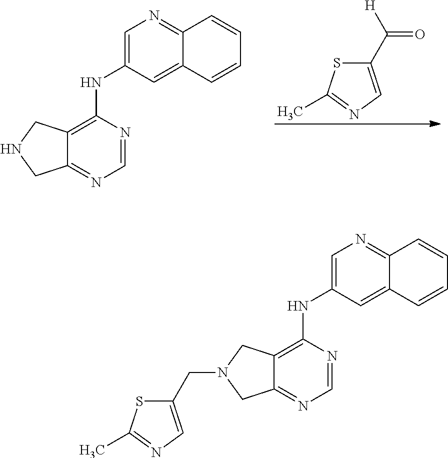

A vial was charged with (6,7-dihydro-5H-pyrrolo[3,4-d]pyrimidin-4-yl)-quinolin-3-yl-amine (10.0 mg, 0.0380 mmol), 2-methyl-thiazole-5-carbaldehyde (5.31 mg, 0.0418 mmol), 1,2-dichloroethane (0.5 mL, 6 mmol) and methanol (0.1 mL, 2 mmol). Sodium triacetoxyborohydride (24.1 mg, 0.114 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-methyl-thiazol-5-ylmethyl)-6,7-dihydro-5H-pyrrolo[3,4-d]pyrimidin-4-yl]-quinolin-3-yl-amine (5.6 mg) as an pale yellow solid.

### Compound 72

### [6-(2-Chloro-benzenesulfonyl)-6,7-dihydro-5H-pyrrolo[3,4-d]pyrimidin-4-yl]-quinolin-3-yl-amine

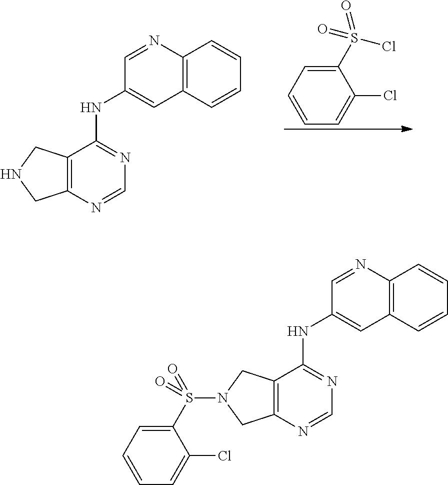

A vial was charged with (6,7-dihydro-5H-pyrrolo[3,4-d]pyrimidin-4-yl)-quinolin-3-yl-amine dihydrochloride (12.000 mg, 0.035691 mmol), 2-chlorobenzenesulfonyl chloride (11.30 mg, 0.05354 mmol), N,N-diisopropylethylamine (31.08 uL, 0.1784 mmol) and acetonitrile (500 uL, 10 mmol). The reaction was stirred for 1 hour at room temperature. The reaction mixture was quenched with saturated NaHCO₃ solution (5 mL) and extracted with EtOAc (5 mL). The organic layer was evaporated to dryness and redissolved in DMSO (1 mL). Reversed-phase HPLC (acetonitrile-water at pH10) gave [6-(2-chloro-benzenesulfonyl)-6,7-dihydro-5H-pyrrolo[3,4-d]pyrimidin-4-yl]-quinolin-3-yl-amine (3.0 mg) as a white solid.

### Compound 77

### [6-(4-Methyl-thiazol-2-ylmethyl)-6,7-dihydro-5H-pyrrolo[3,4-d]pyrimidin-4-yl]-quinolin-3-yl-amine

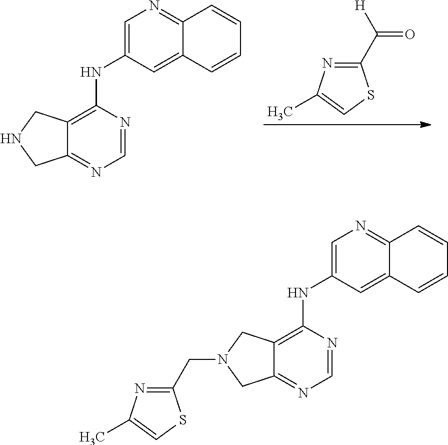

A vial was charged with (6,7-dihydro-5H-pyrrolo[3,4-d]pyrimidin-4-yl)-quinolin-3-yl-amine (10.0 mg, 0.0380 mmol), 4-methyl-thiazole-2-carbaldehyde (5.31 mg, 0.0418 mmol), 1,2-dichloroethane (0.5 mL, 6 mmol) and methanol (0.1 mL, 2 mmol). Sodium triacetoxyborohydride (24.1 mg, 0.114 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(4-methyl-thiazol-2-ylmethyl)-6,7-dihydro-5H-pyrrolo[3,4-d]pyrimidin-4-yl]-quinolin-3-yl-amine (7.2 mg) as a white solid.

### Compound 78

### [6-(3-Methyl-pyridin-4-ylmethyl)-6,7-dihydro-5H-pyrrolo[3,4-d]pyrimidin-4-yl]-quinolin-3-yl-amine

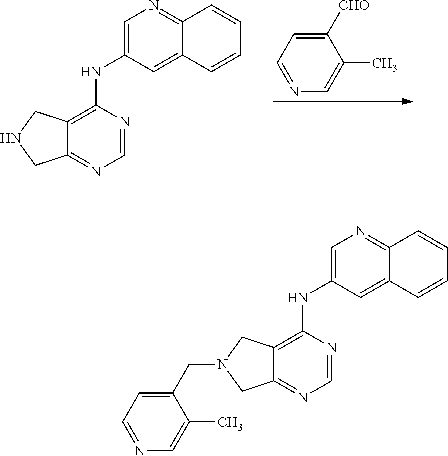

A vial was charged with (6,7-dihydro-5H-pyrrolo[3,4-d]pyrimidin-4-yl)-quinolin-3-yl-amine (10.0 mg, 0.0380 mmol), 3-methyl-pyridine-4-carbaldehyde (5.06 mg, 0.0418 mmol), 1,2-dichloroethane (0.5 mL, 6 mmol) and methanol (0.1 mL, 2 mmol). Sodium triacetoxyborohydride (24.1 mg, 0.114 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave 6-(3-methyl-pyridin-4-ylmethyl)-6,7-dihydro-5H-pyrrolo[3,4-d]pyrimidin-4-yl]-quinolin-3-yl-amine (2.6 mg) as a white solid.

### Compound 80

### [6-(2-Fluoro-benzyl)-6,7-dihydro-5H-pyrrolo[3,4-d]pyrimidin-4-yl]-quinolin-3-yl-amine

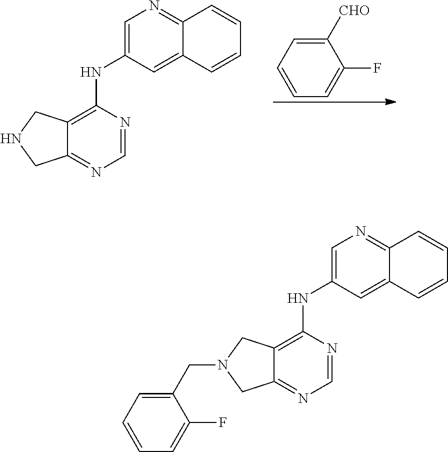

A vial was charged with (6,7-dihydro-5H-pyrrolo[3,4-d]pyrimidin-4-yl)-quinolin-3-yl-amine (15.0 mg, 0.0570 mmol), 2-fluoro-benzaldehyde, (7.78 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-fluoro-benzyl)-6,7-dihydro-5H-pyrrolo[3,4-d]pyrimidin-4-yl]-quinolin-3-yl-amine (1.0 mg) as a white solid.

### Compound 81

### Quinolin-3-yl-(6-thiazol-5-ylmethyl-6,7-dihydro-5H-pyrrolo[3,4-d]pyrimidin-4-yl)-amine

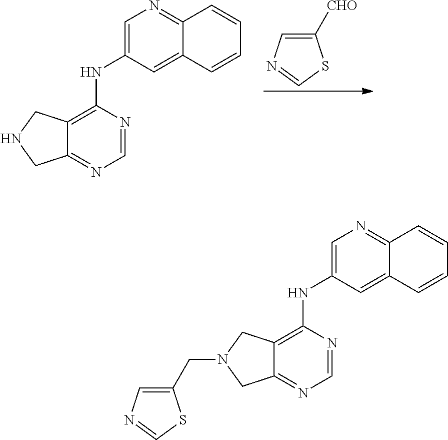

A vial was charged with (6,7-dihydro-5H-pyrrolo[3,4-d]pyrimidin-4-yl)-quinolin-3-yl-amine (15.0 mg, 0.0570 mmol), thiazole-5-carbaldehyde (7.09 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave quinolin-3-yl-(6-thiazol-5-ylmethyl-6,7-dihydro-5H-pyrrolo[3,4-d]pyrimidin-4-yl)-amine (3.0 mg) as a yellow solid.

### Compound 83

### [6-(2-Methoxy-benzyl)-6,7-dihydro-5H-pyrrolo[3,4-d]pyrimidin-4-yl]-quinolin-3-yl-amine

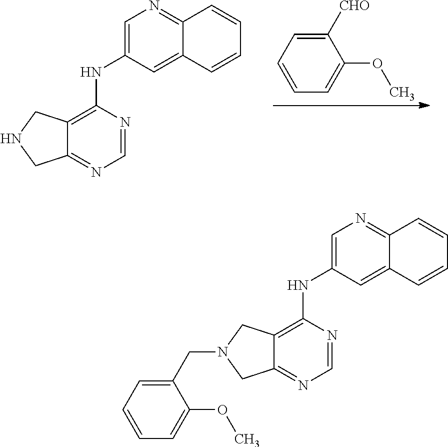

A vial was charged with (6,7-dihydro-5H-pyrrolo[3,4-d]pyrimidin-4-yl)-quinolin-3-yl-amine (15.0 mg, 0.0570 mmol), 2-methoxybenzaldehyde (8.53 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-methoxy-benzyl)-6,7-dihydro-5H-pyrrolo[3,4-d]pyrimidin-4-yl]-quinolin-3-yl-amine (1.1 mg) as a white solid.

### Compound 84

### [6-(2-Chloro-benzyl)-6,7-dihydro-5H-pyrrolo[3,4-d]pyrimidin-4-yl]-quinolin-3-yl-amine

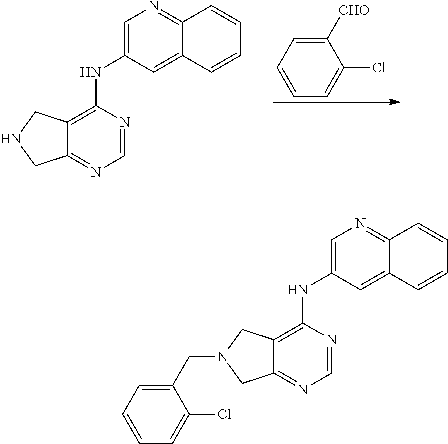

A vial was charged with (6,7-dihydro-5H-pyrrolo[3,4-d]pyrimidin-4-yl)-quinolin-3-yl-amine (15.0 mg, 0.0570 mmol), 2-chlorobenzaldehyde (8.81 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-chloro-benzyl)-6,7-dihydro-5H-pyrrolo[3,4-d]pyrimidin-4-yl]-quinolin-3-yl-amine (1.7 mg) as a white solid.

### Compound 86

### [6-(3-Methyl-furan-2-ylmethyl)-6,7-dihydro-5H-pyrrolo[3,4-d]pyrimidin-4-yl]-quinolin-3-yl-amine

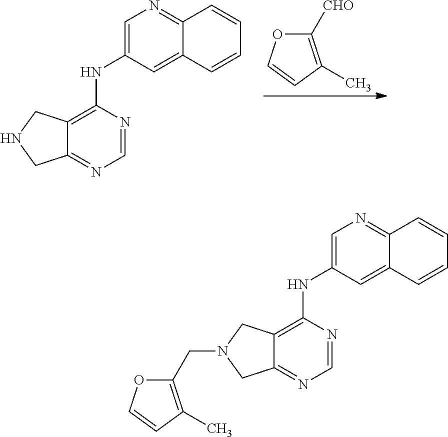

A vial was charged with (6,7-dihydro-5H-pyrrolo[3,4-d]pyrimidin-4-yl)-quinolin-3-yl-amine (15.0 mg, 0.0570 mmol), 3-methyl-furan-2-carbaldehyde (6.90 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(3-methyl-furan-2-ylmethyl)-6,7-dihydro-5H-pyrrolo[3,4-d]pyrimidin-4-yl]-quinolin-3-yl-amine (3.9 mg) as a white solid.

### Compound 87

### [6-(2,3-Dimethyl-benzyl)-6,7-dihydro-5H-pyrrolo[3,4-d]pyrimidin-4-yl]-quinolin-3-yl-amine

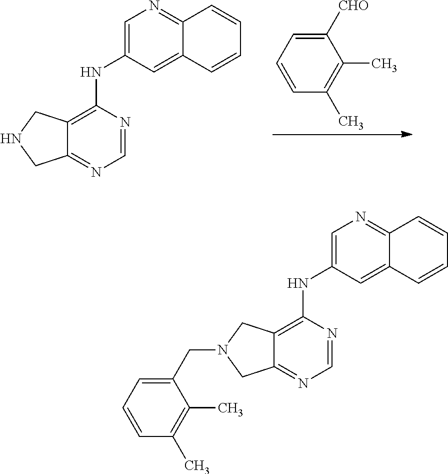

A vial was charged with (6,7-dihydro-5H-pyrrolo[3,4-d]pyrimidin-4-yl)-quinolin-3-yl-amine (15.0 mg, 0.0570 mmol), 2,3-dimethyl-benzaldehyde (8.41 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2,3-dimethyl-benzyl)-6,7-dihydro-5H-pyrrolo[3,4-d]pyrimidin-4-yl]-quinolin-3-yl-amine (4.6 mg) as a white solid.

### Compound 88

### [6-(2,6-Dimethyl-benzyl)-6,7-dihydro-5H-pyrrolo[3,4-d]pyrimidin-4-yl]-quinolin-3-yl-amine

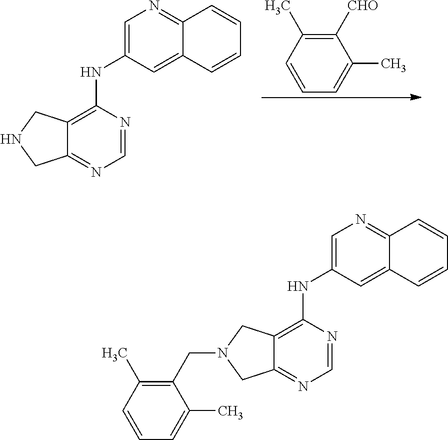

A vial was charged with (6,7-dihydro-5H-pyrrolo[3,4-d]pyrimidin-4-yl)-quinolin-3-yl-amine (15.0 mg, 0.0570 mmol), 2,6-dimethyl-benzaldehyde (8.41 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2,6-dimethyl-benzyl)-6,7-dihydro-5H-pyrrolo[3,4-d]pyrimidin-4-yl]-quinolin-3-yl-amine (2.8 mg) as a white solid.

### Compound 89

### [6-(2,5-Dimethyl-benzyl)-6,7-dihydro-5H-pyrrolo[3,4-d]pyrimidin-4-yl]-quinolin-3-yl-amine

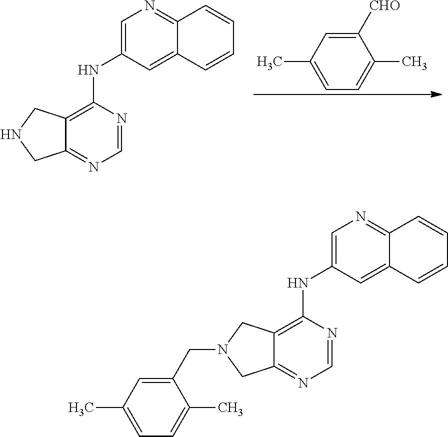

A vial was charged with (6,7-dihydro-5H-pyrrolo[3,4-d]pyrimidin-4-yl)-quinolin-3-yl-amine (15.0 mg, 0.0570 mmol), 2,5-dimethyl-benzaldehyde, (8.41 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2,5-dimethyl-benzyl)-6,7-dihydro-5H-pyrrolo[3,4-d]pyrimidin-4-yl]-quinolin-3-yl-amine (4.7 mg) as a white solid.

### Compound 90

### [6-(2-Ethyl-benzyl)-6,7-dihydro-5H-pyrrolo[3,4-d]pyrimidin-4-yl]-quinolin-3-yl-amine

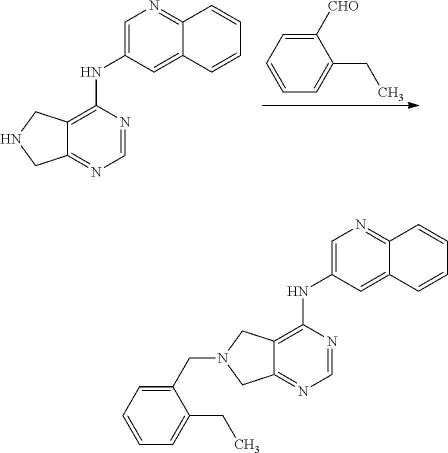

A vial was charged with (6,7-dihydro-5H-pyrrolo[3,4-d]pyrimidin-4-yl)-quinolin-3-yl-amine (15.0 mg, 0.0570 mmol), 2-ethyl-benzaldehyde (8.41 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-ethyl-benzyl)-6,7-dihydro-5H-pyrrolo[3,4-d]pyrimidin-4-yl]-quinolin-3-yl-amine (4.9 mg) as a white solid.

### Compound 91

### [6-(2-Chloro-thiazol-5-ylmethyl)-6,7-dihydro-5H-pyrrolo[3,4-d]pyrimidin-4-yl]-quinolin-3-yl-amine

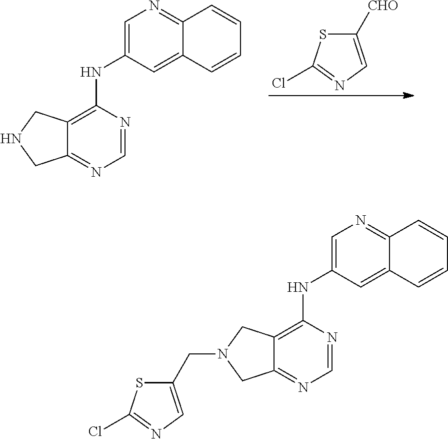

A vial was charged with (6,7-dihydro-5H-pyrrolo[3,4-d]pyrimidin-4-yl)-quinolin-3-yl-amine (100 mg, 0.4 mmol), 2-chloro-thiazole-5-carbaldehyde (61.6 mg, 0.418 mmol), 1,2-dichloroethane (5 mL, 60 mmol) and methanol (1 mL, 20 mmol). Sodium triacetoxyborohydride (241 mg, 1.14 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-chloro-thiazol-5-ylmethyl)-6,7-dihydro-5H-pyrrolo[3,4-d]pyrimidin-4-yl]-quinolin-3-yl-amine (20 mg) as a yellow solid.

### Compound 92

### [6-(2-Methyl-benzyl)-6,7-dihydro-5H-pyrrolo[3,4-d]pyrimidin-4-yl]-quinolin-3-yl-amine

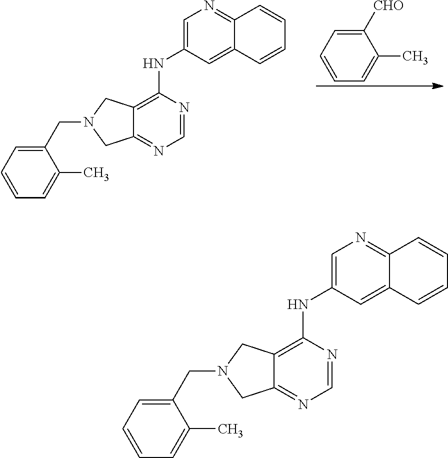

A vial was charged with (6,7-dihydro-5H-pyrrolo[3,4-d]pyrimidin-4-yl)-quinolin-3-yl-amine (15.0 mg, 0.0570 mmol), 2-methylbenzaldehyde (7.53 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-methyl-benzyl)-6,7-dihydro-5H-pyrrolo[3,4-d]pyrimidin-4-yl]-quinolin-3-yl-amine (5.2 mg) as a white solid.

### Compound 94

### (6-Pyridin-4-ylmethyl-6,7-dihydro-5H-pyrrolo[3,4-d]pyrimidin-4-yl)-quinolin-3-yl-amine

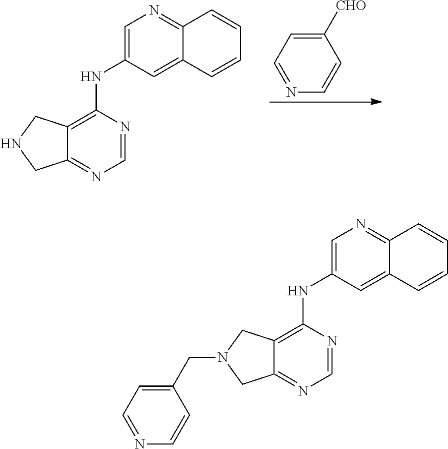

A vial was charged with (6,7-dihydro-5H-pyrrolo[3,4-d]pyrimidin-4-yl)-quinolin-3-yl-amine (15.0 mg, 0.0570 mmol), 4-pyridinecarboxaldehyde (6.71 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave (6-pyridin-4-ylmethyl-6,7-dihydro-5H-pyrrolo[3,4-d]pyrimidin-4-yl)-quinolin-3-yl-amine (2.4 mg) as a white solid.

### Compound 95

### Quinolin-3-yl-[6-(2-trifluoromethyl-benzyl)-6,7-dihydro-5H-pyrrolo[3,4-d]pyrimidin-4-yl]-amine

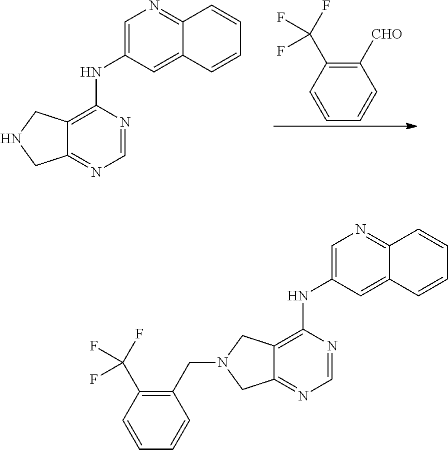

A vial was charged with (6,7-dihydro-5H-pyrrolo[3,4-d]pyrimidin-4-yl)-quinolin-3-yl-amine (15.0 mg, 0.0570 mmol), 2-(trifluoromethyl)benzaldehyde (10.9 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours.

The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave quinolin-3-yl-[6-(2-trifluoromethyl-benzyl)-6,7-dihydro-5H-pyrrolo[3,4-d]pyrimidin-4-yl]-amine (1.5 mg) as a white solid.

### Compound 97

### [6-(2-Ethyl-4-methyl-thiazol-5-ylmethyl)-6,7-dihydro-5H-pyrrolo[3,4-d]pyrimidin-4-yl]-quinolin-3-yl-amine

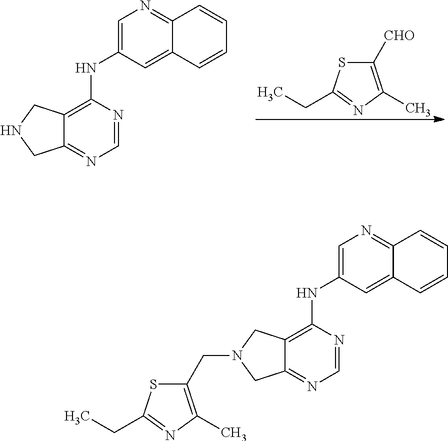

A vial was charged with (6,7-dihydro-5H-pyrrolo[3,4-d]pyrimidin-4-yl)-quinolin-3-yl-amine (10.0 mg, 0.0380 mmol), 2-Ethyl-4-methyl-thiazole-5-carbaldehyde (6.48 mg, 0.0418 mmol), 1,2-dichloroethane (0.5 mL, 6 mmol) and methanol (0.1 mL, 2 mmol). Sodium triacetoxyborohydride (24.1 mg, 0.114 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-ethyl-4-methyl-thiazol-5-ylmethyl)-6,7-dihydro-5H-pyrrolo[3,4-d]pyrimidin-4-yl]-quinolin-3-yl-amine (1.5 mg) as a white solid.

### Compound 98

### Quinolin-3-yl-[6-(2-trifluoromethoxy-benzyl)-6,7-dihydro-5H-pyrrolo[3,4-d]pyrimidin-4-yl]-amine

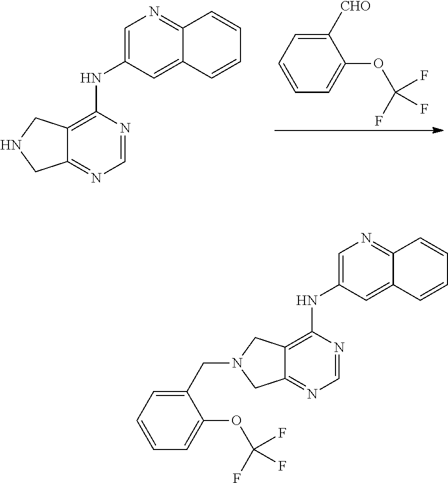

A vial was charged with (6,7-dihydro-5H-pyrrolo[3,4-d]pyrimidin-4-yl)-quinolin-3-yl-amine (15.0 mg, 0.0570 mmol), 2-Trifluoromethoxy-benzaldehyde (11.9 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave quinolin-3-yl-[6-(2-trifluoromethoxy-benzyl)-6,7-dihydro-5H-pyrrolo[3,4-d]pyrimidin-4-yl]-amine (0.7 mg) as a white solid.

### Compound 99

### Quinolin-3-yl-[6-(3-trifluoromethyl-benzyl)-6,7-dihydro-5H-pyrrolo[3,4-d]pyrimidin-4-yl]-amine

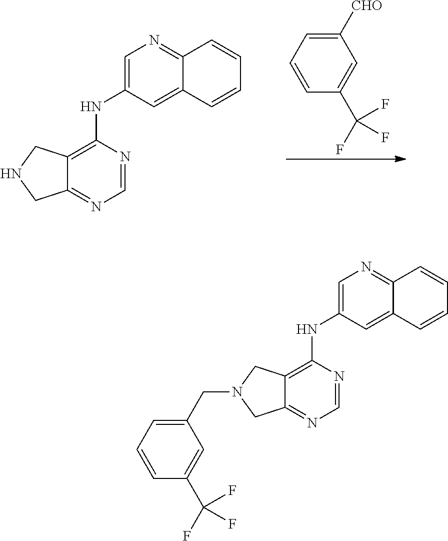

A vial was charged with (6,7-dihydro-5H-pyrrolo[3,4-d]pyrimidin-4-yl)-quinolin-3-yl-amine (15.0 mg, 0.0570 mmol), 3-trifluoromethyl-benzaldehyde (10.9 mg, 0.0627 mmol), 1,2-dichloroethane (0.8 mL, 10 mmol) and methanol (0.2 mL, 4 mmol). Sodium triacetoxyborohydride (36.2 mg, 0.171 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave quinolin-3-yl-[6-(3-trifluoromethyl-benzyl)-6,7-dihydro-5H-pyrrolo[3,4-d]pyrimidin-4-yl]-amine (1.2 mg) as a white solid.

### Compound 100

### [6-(2-Ethoxy-benzyl)-6,7-dihydro-5H-pyrrolo[3,4-d]pyrimidin-4-yl]-quinolin-3-yl-amine

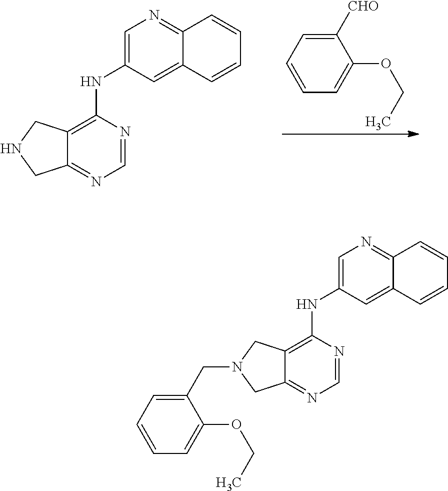

A vial was charged with (6,7-dihydro-5H-pyrrolo[3,4-d]pyrimidin-4-yl)-quinolin-3-yl-amine (12.0 mg, 0.0456 mmol), 2-ethoxy-benzaldehyde, (7.53 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-ethoxy-benzyl)-6,7-dihydro-5H-pyrrolo[3,4-d]pyrimidin-4-yl]-quinolin-3-yl-amine (8.0 mg) as a pale yellow solid.

### Compound 101

### [6-(2,3-Dihydro-benzofuran-7-ylmethyl)-6,7-dihydro-5H-pyrrolo[3,4-d]pyrimidin-4-yl]-quinolin-3-yl-amine

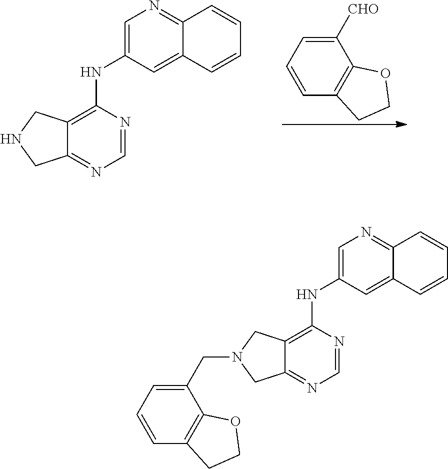

A vial was charged with (6,7-dihydro-5H-pyrrolo[3,4-d]pyrimidin-4-yl)-quinolin-3-yl-amine (12.0 mg, 0.0456 mmol), 2,3-dihydro-benzofuran-7-carbaldehyde (7.43 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2,3-dihydro-benzofuran-7-ylmethyl)-6,7-dihydro-5H-pyrrolo[3,4-d]pyrimidin-4-yl]-quinolin-3-yl-amine (7.0 mg) as a white solid.

### Compound 102

### [6-(2-Methylsulfanyl-benzyl)-6,7-dihydro-5H-pyrrolo[3,4-d]pyrimidin-4-yl]-quinolin-3-yl-amine

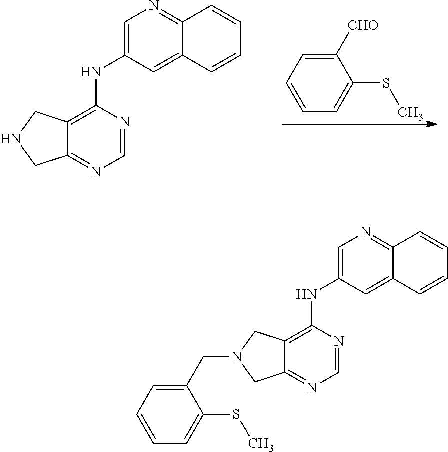

A vial was charged with (6,7-dihydro-5H-pyrrolo[3,4-d]pyrimidin-4-yl)-quinolin-3-yl-amine (12.0 mg, 0.0456 mmol), 2-methylsulfanyl-benzaldehyde (7.63 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-methylsulfanyl-benzyl)-6,7-dihydro-5H-pyrrolo[3,4-d]pyrimidin-4-yl]-quinolin-3-yl-amine (5.6 mg) as a white solid.

### Compound 103

### (6-Benzo[1,3]dioxol-4-ylmethyl-6,7-dihydro-5H-pyrrolo[3,4-d]pyrimidin-4-yl)-quinolin-3-yl-amine

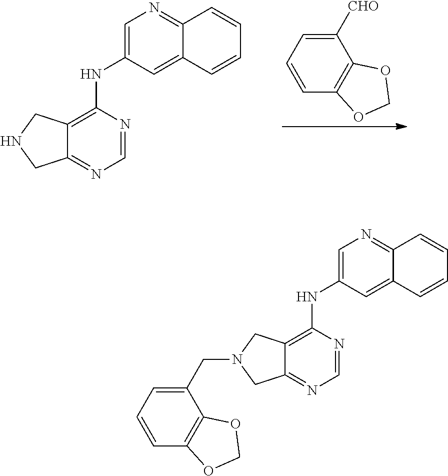

A vial was charged with (6,7-dihydro-5H-pyrrolo[3,4-d]pyrimidin-4-yl)-quinolin-3-yl-amine (12.0 mg, 0.0456 mmol), Benzo[1,3]dioxole-4-carbaldehyde (7.53 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave (6-benzo[1,3]dioxol-4-ylmethyl-6,7-dihydro-5H-pyrrolo[3,4-d]pyrimidin-4-yl)-quinolin-3-yl-amine (8.7 mg) as a white solid.

### Compound 104

### Quinolin-3-yl-[6-(3-trifluoromethoxy-benzyl)-6,7-dihydro-5H-pyrrolo[3,4-d]pyrimidin-4-yl]-amine

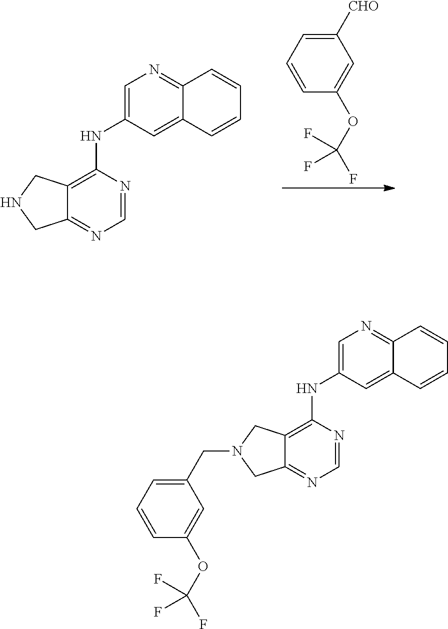

A vial was charged with (6,7-dihydro-5H-pyrrolo[3,4-d]pyrimidin-4-yl)-quinolin-3-yl-amine (12.0 mg, 0.0456 mmol), 3-trifluoromethoxy-benzaldehyde (9.53 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave quinolin-3-yl-[6-(3-trifluoromethoxy-benzyl)-6,7-dihydro-5H-pyrrolo[3,4-d]pyrimidin-4-yl]-amine (7.4 mg) as a white solid.

### Compound 105

### [6-(2-Difluoromethoxy-benzyl)-6,7-dihydro-5H-pyrrolo[3,4-d]pyrimidin-4-yl]-quinolin-3-yl-amine

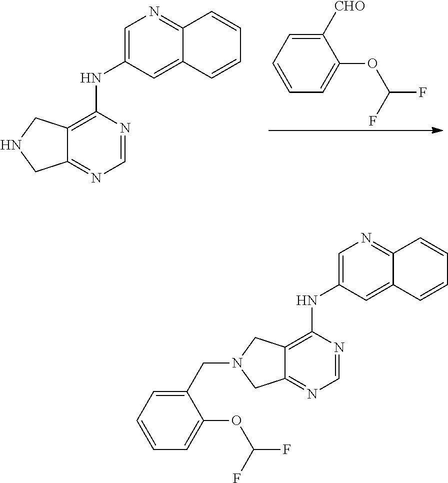

A vial was charged with (6,7-dihydro-5H-pyrrolo[3,4-d]pyrimidin-4-yl)-quinolin-3-yl-amine (12.0 mg, 0.0456 mmol), 2-difluoromethoxy-benzaldehyde (8.63 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-difluoromethoxy-benzyl)-6,7-dihydro-5H-pyrrolo[3,4-d]pyrimidin-4-yl]-quinolin-3-yl-amine (8.2 mg) as a white solid.

### Compound 106

### [6-(3-Methoxy-benzyl)-6,7-dihydro-5H-pyrrolo[3,4-d]pyrimidin-4-yl]-quinolin-3-yl-amine

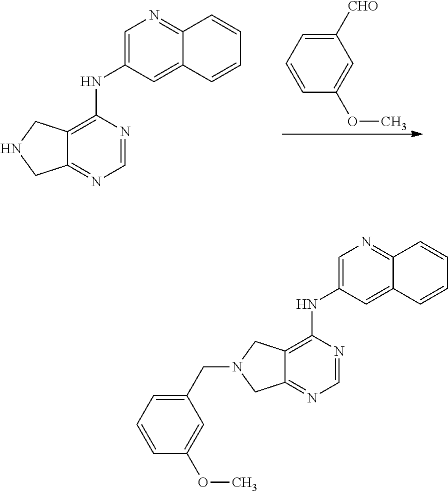

A vial was charged with (6,7-dihydro-5H-pyrrolo[3,4-d]pyrimidin-4-yl)-quinolin-3-yl-amine (12.0 mg, 0.0456 mmol), benzaldehyde, 3-methoxy- (6.82 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(3-methoxy-benzyl)-6,7-dihydro-5H-pyrrolo[3,4-d]pyrimidin-4-yl]-quinolin-3-yl-amine (2.8 mg) as a white solid.

### Compound 107

### [6-(2,3-Difluoro-benzyl)-6,7-dihydro-5H-pyrrolo[3,4-d]pyrimidin-4-yl]-quinolin-3-yl-amine

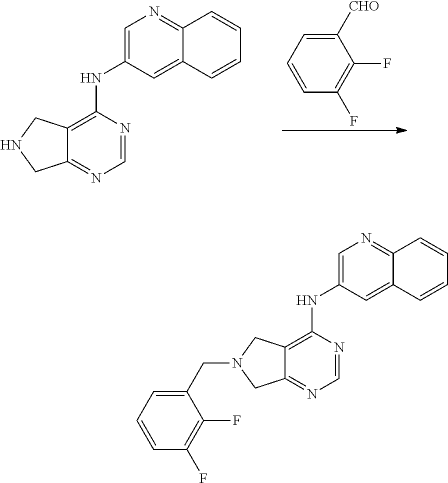

A vial was charged with (6,7-dihydro-5H-pyrrolo[3,4-d]pyrimidin-4-yl)-quinolin-3-yl-amine (12.0 mg, 0.0456 mmol), 2,3-difluoro-benzaldehyde (7.12 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2,3-difluoro-benzyl)-6,7-dihydro-5H-pyrrolo[3,4-d]pyrimidin-4-yl]-quinolin-3-yl-amine (7.0 mg) as a white solid.

### Compound 109

### [6-(2-Isopropoxy-benzyl)-6,7-dihydro-5H-pyrrolo[3,4-d]pyrimidin-4-yl]-quinolin-3-yl-amine

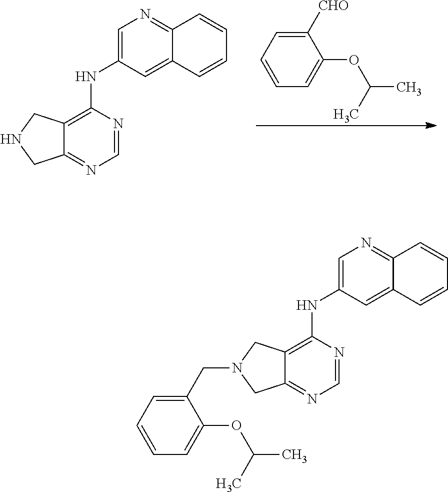

A vial was charged with (6,7-dihydro-5H-pyrrolo[3,4-d]pyrimidin-4-yl)-quinolin-3-yl-amine (12.0 mg, 0.0456 mmol), 2-isopropoxybenzaldehyde (8.23 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-isopropoxy-benzyl)-6,7-dihydro-5H-pyrrolo[3,4-d]pyrimidin-4-yl]-quinolin-3-yl-amine (8.3 mg) as a white solid.

### Compound 110

### [6-(1H-Indol-7-ylmethyl)-6,7-dihydro-5H-pyrrolo[3,4-d]pyrimidin-4-yl]-quinolin-3-yl-amine

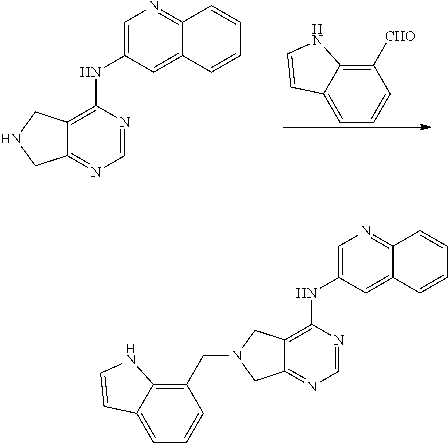

A vial was charged with (6,7-dihydro-5H-pyrrolo[3,4-d]pyrimidin-4-yl)-quinolin-3-yl-amine (12.0 mg, 0.0456 mmol), 1H-indole-7-carbaldehyde (7.28 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(1H-indol-7-ylmethyl)-6,7-dihydro-5H-pyrrolo[3,4-d]pyrimidin-4-yl]-quinolin-3-yl-amine (4.2 mg) as a white solid.

### Compound 111

### [6-(3-Methyl-benzyl)-6,7-dihydro-5H-pyrrolo[3,4-d]pyrimidin-4-yl]-quinolin-3-yl-amine

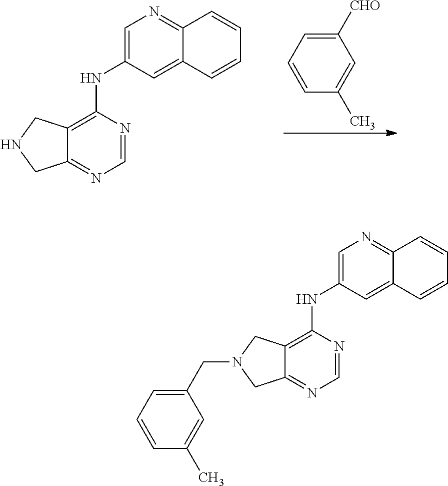

A vial was charged with (6,7-dihydro-5H-pyrrolo[3,4-d]pyrimidin-4-yl)-quinolin-3-yl-amine (12.0 mg, 0.0456 mmol), 3-methylbenzaldehyde (6.02 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(3-methyl-benzyl)-6,7-dihydro-5H-pyrrolo[3,4-d]pyrimidin-4-yl]-quinolin-3-yl-amine (5.2 mg) as an off-white solid.

### Compound 112

### [6-(2-Cyclopropyl-benzyl)-6,7-dihydro-5H-pyrrolo[3,4-d]pyrimidin-4-yl]-quinolin-3-yl-amine

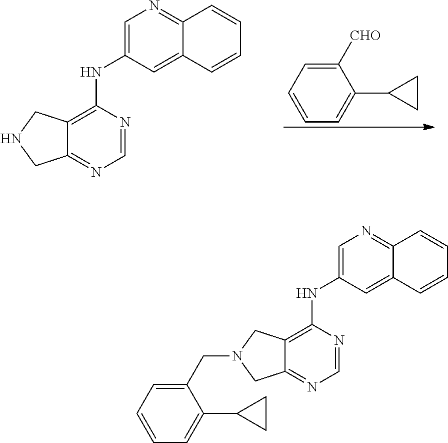

A vial was charged with (6,7-dihydro-5H-pyrrolo[3,4-d]pyrimidin-4-yl)-quinolin-3-yl-amine (12.0 mg, 0.0456 mmol), 2-cyclopropyl-benzaldehyde (7.33 mg, 0.0501 mmol), 1,2-dichloroethane (0.6 mL, 8 mmol) and methanol (0.1 mL, 3 mmol). Sodium triacetoxyborohydride (29.0 mg, 0.137 mmol) was added, and the reaction stirred at room temperature for 2 hours. The reaction mixture was quenched with saturated NaHCO₃ solution (5 mL) and extracted with DCM (5 mL). The organic layer was evaporated to dryness and redissolved in DMSO (1 mL). Purification using reversed-phase HPLC (acetonitrile-water at pH10) gave [6-(2-cyclopropyl-benzyl)-6,7-dihydro-5H-pyrrolo[3,4-d]pyrimidin-4-yl]-quinolin-3-yl-amine (4.1 mg) as a white solid.

### Assays

### hFAAH Microsome Collection Procedure

Human cell line T84 (human colon epithelial cells), which expresses endogenous hFAAH, or HEK293-TRex cells (Invitrogen) stably transfected with hFAAH in the pcDNA5-Tet-off vector are cultured in medium containing DMEM, 10% FBS penicillin/streptomycin, glutamax, 200 μg/ml hygromycin and 0.5 μg/ml blasticidin. Cell collection is done 24 h after induction with doxycycline by first washing the cells with cold PBS and then incubating them with Versene before centrifugation. Cell pellets are then stored at −80° C. until needed. For homogenization, the cell pellets are thawed on ice at room temperature and resuspended in homogenization buffer (50 mM HEPES (pH 7.4), 1 mM EDTA, 1 μM Pepstatin A, 100 μM Leupeptin, 0.1 mg/mL aprotinin). Cell suspensions are then homogenized on ice using the Polytron 1200 C at setting 6 for three 30-second intervals with 30-second rests. The suspension is centrifuged at 1000 g for 10 minutes at 4° C. and the supernatant is collected and further centrifuged at 24000 rpm for 30 minutes at 4° C. using an ultracentrifuge. Pellets are resuspended by adding in cold microsomal buffer (50 mM HEPES (pH 7.4) and 1 mM EDTA) and sheared through a 23-gauge needle five times, keeping the suspension on ice. Protein concentrations are determined using the BCA assay and aliquoted preparations are stored at −80° C. until needed.

### Compound IC₅₀ Determination

Fluorimetric Assay with hFAAH Microsomes

Compound potency against hFAAH is determined using an enzymatic assay with a fluorescence readout. Briefly, experiments were carried out in a 96-well plate format (Corning Costar, # 3370) with a total well volume of 160 μL with components added in the following order: assay buffer (50 mM HEPES (pH 7.4), 1 mM EDTA, 1.4 mg/mL BSA), compound solutions (7 different concentrations per compound in duplicate), microsomal enzyme preparation (10 μg per well) and substrate [AA-AMC (arachadonyl 7-amino 4-methyl coumarin amide), 2 μM]. After a brief shaking, a kinetic read of the plate is obtained using a Tecan Safire II in kinetic mode for 275 cycles with excitation and emission wavelengths of 355 and 460 nm, respectively. Raw data is then processed and analyzed using Assay Explorer and GraphPad Prism.

Radioactive Assay with hFAAH Microsomes

Compound potency against hFAAH is determined using an enzymatic assay with a radiometric readout. Briefly, experiments are carried out in 1.5 mL vials with a total well volume of 200 μL with components added in the following order: assay buffer (50 mM HEPES (pH 7.4), 1 mM EDTA, 1 mg/mL BSA), compound solutions (6 different concentrations per compound in triplicate), microsomal enzyme preparation (10 μg per well) and substrate (AEA with ³H-AEA tracer, 1 μM). After a 30 minute incubation, 400 μL of CH₃OH/CHCL₃ 1/1 (v/v) solution is added to each tube, reactions are vortexed and centrifuged before extracting 300 μL of the aqueous layer, mixing it with 5 mL of scintillation fluid before counting the amount of radioactivity present in a liquid scintillation counter. Raw data is then processed and analyzed using GraphPad Prism.

In alternative FAAH assays, ¹⁴C-anandamide may be utilized or anandamide [ethanolamine-1-³H] and incubated with microsomes or cell membranes from liver cells or cell lines. The reaction can be monitored by differential absorption of the substrate and its products to charcoal (L. Boldrup, et al., (2004) J. Biochem. Biophys. Methods 60, 171-177; S. Wilson, et al., (2003) Anal. Biochem. 318, 270-275). A fluorescent assay utilizing substrate decanoyl 7-amino-4-methyl coumarin (D-AMC), that is cleaved to the fluorescent molecule 7-amino-4-methyl coumarin (AMC) has also been described (K. L. Kage, et al., (2006) J. Neurosci. Methods (2006) November 1 [E pub ahead of print: doi:10.1016/j.jneumeth.2006.10.006]).

### Neuropathic Pain Models

### Chronic Constriction Injury Model (CCI or Bennett Model):

The CCI model is performed according to the method described by Bennett and Xie, Pain, 33:87-107, 1988. Briefly, under isoflurane anesthesia, the right sciatic nerve is exposed at mid-thigh level via blunt dissection through the biceps femoris. Proximal to the bifurcation of the sciatic nerve, about 7 mm of nerve is freed of adhering tissue and 4 loose ligatures of 4.0 chromic gut are tied around the nerve. Spacing between ligatures is approximately 1 mm. The wound is closed in layers, and the skin closed with staples or non-silk sutures. Sham operated animals are treated identically with the exception that the sciatic nerve will not be ligated. Mechanical allodynia, cold allodynia, or thermal hyperalgesia testing occur 7-21 days post surgery.

Spinal Nerve Transection (SNT or Chung model):

The SNT model will be performed according to the method described by Kim and Chung, Pain 50:355-363, 1992. Under isoflurane anesthesia, a longitudinal incision is made at the lower lumbar and sacral levels, exposing paraspinal muscles on the left side. The location of the incision is determined by the position of the L5 spinous process. The paraspinal muscles are isolated and removed from the level of the L4 spinous process to the sacrum. This opens up the space ventrolateral to the articular processes, dorsal to the L6 transverse process, and medial to the ileum. Remaining connective tissues and muscles are removed. Under a dissecting microscope, the L6 transverse process, which covers the L5 spinal nerve, is removed. Due to their close proximity, the L4 and L5 spinal nerves may need to be separated to fully expose the L5 spinal nerve for ligation using extra caution not to damage the L4 nerve during this process. Animals that exhibit L4 nerve damage as evidenced by paw drop post-anesthesia are not included in studies. Once the L5 spinal nerve is exposed, the nerve is ligated with 6-0 silk. Alternatively, the spinal nerve is cut distal to the ligation site. If a more complete neuropathy is required, then the L6 spinal nerve may also be ligated using the procedure described above. Sham operated animals are treated identically with the exception that the nerves will not be ligated/transected. Following spinal nerve ligation, hemostasis is confirmed, the muscles are sutured in layers, and the skin is closed with staples or non-silk sutures. Mechanical allodynia, cold allodynia, or thermal hyperalgesia testing occur 7-21 days post surgery.

### Chemotherapy-Induced Painful Neuropathy:

Chemotherapy neuropathy is induced by i.p. administration of 1 mg/kg Taxol administered once/day on 4 alternating days (total dose=4 mg/kg) (Polomano et al., Pain, 94:293-304, 2001). Mechanical allodynia, cold allodynia, or thermal hyperalgesia testing occur 9-30 days post day 1 of Taxol administration.

### Inflammatory Pain Models

### Formalin Model:

Test compounds are administered at various times prior to intraplantar administration of formalin. A dilute solution of formalin (50 μL of 2.5% formaldehyde/saline) is administered s.c. into the plantar surface of the left hind paw under light restraint. Immediately following injection, animals are placed on a mesh stand inside a clear observation chamber large enough to allow for free movement of the animals during the study. Behaviors are scored using manual scoring or automated scoring.

Manual scoring: Using a three channel timer, the observer records the time (t in seconds) of decreased weight-bearing (t₁), paw lifting (t₂), and licking/biting/shaking (t₃). Results are weighted according to the method of Dubuisson and Dennis, Pain, 4:161-174, 1977, using the formula t₁+2t₂+3t₃/180 where 180 s is the evaluation time for each increment. Behaviors are acquired in alternating 3 min increments starting at time=0 min (i.e. 0-3 min, 6-9 min etc.) and ending at 60 min.

Automated scoring: A small metal band weighing 0.5 g is placed on the left paw. Formalin is administered and the animal placed unrestrained inside an observation chamber over an electromagnetic detector system (Automated Nociception Analyzer, University of California, San Diego). The number of paw flinches are electronically recorded.

### Complete Freund's Adjuvant Model (CFA):

Animals receive an s.c. injection of 100 μL complete Freund's adjuvant containing 100 μg Mycobacterium tuberculosis strain H37Ra into the plantar surface of the right hind paw under isoflurane anesthesia. Swelling and inflammation are visible within 1 h after administration. Mechanical allodynia or thermal hyperalgesia testing start 24 h post CFA administration.

### Carageenan:

Animals receive a subcutaneous injection of 100 μL of either 2% carrageenan or saline (controls) into the plantar surface of the right hind paw under isoflurane anesthesia. Swelling and inflammation are visible within 1 h after administration. Mechanical allodynia or thermal hyperalgesia testing start 3-24 h post carageenan administration (Hargreaves et al., Pain, 32:77-88, 1988).

### Visceral Pain Models

### Colo-Rectal Distension (CRD):

Prior to induction of the model, animals are deprived of food but allowed access to water ad libitum for 16 h prior to the induction of the model. A 5 cm latex balloon is attached to a barostat system composed of a flow meter and pressure control program by a length of tubing. Under isoflurane anesthesia, the balloon is inserted into the distal colon via the anus at a distance of 5 cm from the anus and taped to the base of the tail. Post-anesthesia, the animal is placed unrestrained into a clean polypropylene cage and allowed to acclimate for 30 mins. The balloon is progressively inflated from 0-75 mmHg in 5 mm increments every 30 s. The colonic reaction threshold is defined as the pressure inducing the first abdominal contraction. Abdominal contraction indicative of visceral pain correlates with hunching, hump-backed position, licking of the lower abdomen, repeated waves of contraction of the ipsilateral oblique musculature with inward turning of the ipsilateral hindlimb, stretching, squashing of the lower abdomen against the floor (Wesselman, Neurosci. Lett., 246:73-76, 1998).

### Acetic Acid Writhing Test:

A 0.6% solution of acetic acid (10 ml/kg) is administered i.p. to rats and the number of abdominal constrictions over 30 min are counted.

### Behavioral Testing

### Mechanical Testing:

Mechanical allodynia testing is performed using the up-down method of Dixon, Ann. Rev. Pharmacol. Toxicol. 20:441-462, 1980, modified for mechanical thresholds by Chaplan et al., J. Neurosci. Methods 53:55-63, 1994. Testing is performed during the day portion of the circadian cycle (7:00-19:00). Animals are placed in separate plastic enclosures with a mesh bottom which allowed for full access to the paws. For all tests, animals are acclimated to the apparatus for at least 15 min prior to testing or until cage exploration and major grooming activities have ceased. The area tested will be the mid-plantar hind paw. The paw is touched with 1 of a series of 8 von Frey hairs (Stoelting, Wood Dale, Ill.) with logarithmically incremental stiffness (0.4, 0.6, 1.4, 2, 4, 6, 8, and 15 g). Each von Frey hair is presented perpendicularly to the plantar surface with sufficient force to cause slight buckling against the paw and held for approximately 6-8 s. Stimulation is presented at intervals of several seconds, allowing for apparent resolution of any behavioral responses to previous stimuli. A positive response will be noted if the paw is sharply withdrawn. Flinching immediately upon removal of the hair will also be considered a positive response. Ambulation will be considered an ambiguous response and in such cases, the stimulus will be repeated.

To determine the 50% withdrawal threshold, testing will be initiated with the 2 g fiber (the middle fiber in the series). Fibers will be presented in a consecutive fashion whether ascending or descending. In the absence of a paw withdrawal response to the initially selected fiber, the next highest fiber is presented. In the event of a paw withdrawal, the next weaker fiber is presented. The optimal threshold calculation by this method requires 6 responses in the immediate vicinity of the 50% withdrawal threshold. Counting of the critical 6 data points will not begin until the response threshold is first crossed at which time the 2 responses straddling the threshold will be designated as the first 2 responses of the series of 6. Four additional responses to the continued presentation of the fibers constituted the remaining 4 responses.

In cases where continuous positive or negative responses are observed to the exhaustion of the fiber set, values of 15 g and 0.25 g are assigned, respectively.

The range of fibers tested in this paradigm have not been shown to cause tissue damage although prolonged stimulation over short time intervals may result in sensitization and/or habituation, scenarios which would lead to decreased or increased thresholds, respectively. Therefore, there is a minimum 1 h interval between testing sessions with no more than 4 testing sessions per day. For testing intervals, animals are returned to their cages following all testing sessions. Testing sessions will last no longer than 1 h. No two testing sessions will occur on consecutive hours. To minimize distress, mechanical allodynia testing is conducted no more than 4 times per day.

### Thermal Testing:

To measure heat thermal hyperalgesia, an Ugo Basile radiant heat source (I.R. intensity of 40) will be provided by a light bulb focused onto the plantar surface of the paw (Hargreaves et al., Pain 32:77-88, 1988). Paw withdrawal latencies are defined as the time it takes for the animal to remove its paw from the heat source. To ensure that no tissue damage occurs, all tests will have a 20 sec cutoff even when the animal does not withdraw its paw away from the heat stimulation. The test consists of 3 measurements of the same paw, with a minimum 5 minute intervals between each determination. To minimize distress, thermal testing is conducted no more than 3 times per day.

### Cold Testing:

To measure cold allodynia, a drop of acetone is applied to the plantar surface of the paw through the underside of the grating on which the animals are standing using a 50 μL Hamilton syringe. The process is performed 5 times with a 3 min interval between each time. Vigorous shaking will be recorded as a positive response. The acetone drop test is conducted no more than 5 times over the course of a study (including the pre-surgery baseline test) and no more than once per day (Kotinen et al., Pain 80:341-346, 1999).

### Neuropathic Pain Measurements Using Chung Model

Under pentobarbital anesthesia (60 mg/kg, i.p.), rats are placed in a prone position on a flat, sterile surface. A midline incision from L4-S2 is made and the left paraspinal muscles are separated from the spinous processes. The L5 and L6 spinal nerves are tightly ligated with a 4-0 silicon-treated silk suture, according to the method described by Kim and Chung, Pain, 50:355-363, 1992. The L4 spinal nerve is carefully preserved from being surgically injured. The skin is closed with wound clips and animals are returned to their home cages. Rats exhibiting prolonged postoperative neurological deficits or poor grooming are excluded from the experiments. The animals are assessed for response to noxious mechanical stimuli by determining paw withdrawal threshold (PWT), as described below, prior to surgery (baseline), then immediately prior to and at various time points after being administered with a compound of this invention (30 mg/kg) in the left rear paw of the animal. Additionally, other animals may also be assessed for thermal or mechanical hyperalgesia, as described below.

Assessment of Tactile Allodynia: To assess tactile allodynia, rats are placed in clear, Plexiglas compartments with a wire mesh floor and allowed to habituate for a period of at least 15 minutes. After habituation, a series of von Frey monofilaments are presented to the plantar surface of the left (operated) foot of each rat. The series of von Frey monofilaments consists of six monofilaments of increasing diameter, with the smallest diameter fiber presented first. Five trials are conducted with each filament with each trial separated by approximately 2 minutes. Each presentation lasts for a period of 4-8 seconds or until a nociceptive withdrawal behavior is observed. Flinching, paw withdrawal or licking of the paw are considered nociceptive behavioral responses.

Response to Thermal Stimuli as an Assessment of Thermal Hyperalgesia: The plantar test can be used to assess thermal hyperalgesia. For this test, hind paw withdrawal latencies to a noxious thermal stimulus are determined using a plantar test apparatus (commercially available from Ugo Basile of Italy) following the technique described by Hargreaves et al., Pain 32: 77-88, 1988. The maximum exposure time is set at 32 seconds to avoid tissue damage and any directed paw withdrawal from the heat source is taken as the end point. Three latencies are determined at each time point and averaged. Only the affected (ipsilateral) paw is tested. An increase latency of paw withdrawal demonstrates reversal of hyperalgesia.

Response to Mechanical Stimuli as an Assessment of Mechanical Hyperalgesia: The paw pressure assay can be used to assess mechanical hyperalgesia. For this assay, hind paw withdrawal thresholds (PWT) to a noxious mechanical stimulus are determined using an analgesymeter (Model 7200, commercially available from Ugo Basile of Italy) as described in Stein et al., Pharmacol. Biochem. Behav. 31:451-455, 1988. The maximum weight that can be applied to the hind paw is set at 250 g and the end point is taken as complete withdrawal of the paw. PWT is determined once for each rat at each time point and only the affected (ipsilateral) paw is tested.

### Parkinson's Disease:

Experiments are conducted as described in McCall et al; J. Pharmacol. Exp. Ther. 2005 314(3):1248-1256.

### Locomotor Activity Measurements in Reserpinized Rats

Rats pretreated with reserpine and the dopamine synthesis inhibitor α-methyl-para-tyrosine (AMPT) are akinetic and cataleptic. These effects can be reversed by L-dopa. Reserpine/AMPT-treated rats have been used as a model of dopamine depletion to mimic parkinsonian conditions. Rats weighing 200 to 250 g are used in this assay. Animals are pretreated with reserpine (5 mg/kg s.c., 18 h prior) and AMPT (100 mg/kg s.c., 1 h prior) before the experiment. The animals are injected with the test compound or saline, and locomotor activity is measured using animal activity monitors. Data can be presented as horizontal counts expressed as percentage of saline-treated controls and comparisons can be done at discrete 10-min intervals, comparing vehicle to individual doses of test compound.

### Turning in 6-Hydroxydopamine (6-OHDA)-Lesioned Rats

Unilateral 6-OHDA injections into the substantia nigra cause selective destruction of dopamine neurons, leading to supersensitivity of the dopamine receptors in the caudate putamen on the injected side. In these animals, dopamine receptor agonists cause contralateral turning. Rats (225-280 g) are pretreated with desmethylimipramine 25 mg/kg i.p. 1 h before surgery. They are anesthetized with Chloropent given at 3 ml/kg i.p. and placed in a stereotaxic apparatus with the incisor bar raised to 4 mm (smaller rats) or 5 mm (larger rats). A small hole is drilled through the skull and a 30-gauge stainless tubing is lowered to the right substantia nigra using the following coordinates: for smaller rats, AP, −1.5 mm; L, +1.8 mm; and V, −8.0 mm; for larger rats, AP, −2.8 mm; L, +2.0 mm; and V, −8.0 mm. 6-OHDA, hydrogen bromide solution is injected into the substantia nigra at 12 μg/2 μl free base) in 0.9% saline/0.1% ascorbic acid at 1 μl/min, using a syringe pump. Two weeks after surgery, the effects of the lesions are tested by monitoring the turning rate of rats given 0.5 mg/kg s.c. apomorphine HCl in 0.9% saline/0.1% ascorbic acid. Total turns are recorded at 10-min intervals in automated monitors. Each rat is connected by a lightweight harness and tether to a rotometer at the top of a clear plastic cylindrical cage. Rats can be used for test compound experiments if they have at least 30 turns/10 min (range 30-160 turns/10 min) in this screen.

### MPTP-Lesioned Monkeys

Cynomolgus monkeys (Macaca fascicularis), weighing approximately 3 kg each, are treated with MPTP i.v. at different dosages until variable, but stable, parkinsonian features occur. During the period of peak test compound effect, behavioral responses are scored every 30 min using an MPTP monkey disability scale and locomotor activity is recorded every 15 min by photocells mounted in the cages. Results obtained with L-dopa/benserazide 50:12.5 mg/kg and 0.1 mg/kg apomorphine can be used as reference standards.

### Anxiety:

### Elevated Plus Maze

The elevated plus maze test is used to assess the anxiolytic properties of test compounds. This test can be conducted as described in Walf, A. A. and Frye, C. A. Nature Protocols 2: 322-328 (2007). Briefly, rats are injected with vehicle or test compounds at least 15 minutes before being placed on the center platform of the maze. The maze consists of 4 arms, 2 open and 2 closed, arranged in a cross pattern with a central platform. The number of entries into an open arm along with the time spent in the open arms are recorded with a video-tracking device.

### Light-Dark Exploration

The light dark exploration test is used to evaluate anxiety in mice. The light dark paradigm in mice is based on a conflict between the innate aversion to brightly illuminated areas and the spontaneous exploratory activity. This assay can be conducted as described in Griebel, G. et al J. Pharmacol. Exp. Ther. 2002 301:333-345. Briefly, the test apparatus consists of two polyvinylchloride boxes covered with Plexiglas. One of these boxes is darkened. A desk lamp placed 20 cm above the lit box and a neon tube fixed on the ceiling provide the room illumination so that the light intensity in the center of the illuminated box is 1000 lux. An opaque plastic tunnel separates the dark box from the illuminated one. At the beginning of the experiment, the test mouse is placed in the illuminated box, facing the tunnel. Recording starts when the animal enters the tunnel for the first time. The following parameters can be monitored during a 4-min period: 1) time spent by mice in the lit box; 2) attempt at entry into the lit box followed by avoidance responses (this includes stretched attend posture; the mouse stretches forward and retracts to original position); 3) total number of tunnel crossings; and 4) activity in the lit box. Experiments are performed at least 15 minutes after administration of the test compounds.

### Marble Burying Test

The marble burying test is an anxiety assay conducted in mice. It can be run as described in Chaki, S. et al J. Pharmacol. Exp. Ther. 2003 304:818-826. Briefly, mice are treated with vehicle or test compounds at least 15 minutes prior to the experiment. Mice are then individually placed in transparent, polycarbonate cages containing a 5-cm layer of sawdust and 24 glass marbles (1.5 cm in diameter) evenly spaced against the wall of the cage. Thirty minutes later the animals are removed from the cages and the number of marbles at least two-thirds buried in the sawdust are recorded.

### Depression:

### Forced Swimming Test

The forced swimming test is a model of behavioral despair and it is used to assess the anti-depressant like properties of test compounds. This assay can be run as described in Chaki, S. et al J. Pharmacol. Exp. Ther. 2003 304:818-826 and Detke, M. J. et al Psychopharmacology 1995 121:66-72. A time-sampling technique is used to score several types of behavior (immobility, swimming, climbing). This method has previously been shown to be reliable and valid for detecting effects of different antidepressant drugs (Detke, M. J. et al Psychopharmacology 1995 121:66-72). Swimming sessions are conducted by placing rats in cylinders containing 25° C. water, 30 cm deep, so that rats cannot support themselves by touching the bottom with their feet. Two swimming sessions are conducted: an initial 15-min pretest followed 24 h later by a 5-min test. Test compounds or vehicle are administered during the period between these two sessions. Test sessions are videotaped from the front of the cylinders for later scoring. At the end of each 5-s period during the test session, the scorer rates the rat's behavior as one of the following three behaviors: 1) immobility, floating in the water without struggling, and making only movements necessary to keep its head above water; 2) swimming, making active swimming motions between quadrants of the cylinder, more than necessary to merely keep the head above water, moving around in the cylinder; and 3) climbing movements with forepaws in and out of the water, usually directed against the walls.

### Tail Suspension Test

The tail suspension test is a model of behavioral despair and it is used to assess the anti-depressant like properties of test compounds. This assay can be run as described in Huges, Z. A. et al Neuropharmacology 2008 54:1136-1142. Following treatment with test compounds or vehicle at least 15 minutes before the test, mice are suspended upside down by the tail using adhesive laboratory tape, to a flat metal bar connected to a strain gauge within a tail suspension chamber. The time spent immobile during a 6-min test session is automatically recorded.

### Emesis:

### Conditioned Gaping

Conditioned gaping is a model conducted in rats for the anticipatory nausea occurring in chemotherapy-treated patients. This test can be conducted as described in Rock, E. M. et al; Psychopharmacology 2008 196:389-395. Briefly, rats are conditioned using four conditioning trials at 72 h intervals by injecting them with LiCl (127 mg/kg i.p.) immediately before placing them in a distinctive context (i.e. a chamber laced with vanilla smell). For testing compounds, pre-conditioned rats are separated in different groups which are injected with vehicle or different doses of the compound tested at least 15 minutes prior to testing. Testing is conducted by placing the animals in the distinctive context and monitoring their orofacial responses for gaping reactions for up to 15 minutes. The orofacial responses of the animals are video-recorded for scoring.

### Cisplatin-Mediated Emesis

Cisplatin is known to induce nausea in cancer patients. Cisplatin can be injected in ferrets to induce vomiting to produce a model in which compounds can be tested to ascertain whether they can diminish nausea and vomiting. This test can be conducted as described in Van Sickle M. D. et al Am. J. Physiol. Gastrointest. Liver Physiol. 285: G566-G576, 2003. Briefly, ferrets are anesthetized with halothane and maintained at 1.5-2.0%. Vehicle or test compounds are administered at least 15 min before the emetic agent cisplatin. A small incision is made to expose the left jugular vein for the administration of cisplatin (10 mg/kg iv). The incision is closed and then the unanaesthetized ferret is observed for 3 h or for 1 h after the last emetic episode to count the number of episodes of retching (defined by rhythmic abdominal contractions with an open mouth) and vomiting (defined by retching with the expulsion of saliva and gastric juices).

### Multiple Sclerosis

### Experimental Autoimmune Encephalitis Model (EAE)

The EAE model is a model of multiple sclerosis. This assay can be conducted as described in Mead, R. J. et al J. Immunology 2002 168:458-465. Briefly, rats are immunized in each hind footpad with 50 μl of a 1:1 emulsion of 1 mg/ml guinea pig myelin basic protein (gpMBP) in PBS and CFA containing 4 mg/ml Mycobacterium tuberculosis H37 Ra. Animals are weighed daily and monitored for clinical signs of disease, scored as follows: 0, no clinical signs; 0.5, tail weakness; 1, tail atony; 1.5, tail atony and abnormal gait; 2, hind limb weakness; 2.5, complete paralysis of one hind limb; 3, complete paralysis of both hind limbs; 4, moribund. Test compounds or vehicle are administered as appropriate during the experiment.

### Chronic Relapsing Experimental Allergic Encephalomyelitis (CREAE)

The CREAE model is a model of multiple sclerosis where the mice also develop limb spasticity and tremor. This model can be conducted as described in Ligresti, A. et al Br. J. Pharmacol. 2006 147:83-91 or in Baker, D. et al Nature 2000 404:84-87. Briefly, limb spasticity in CREAE mice is assessed as follows. Spasticity is induced in ABH mice following the induction of experimental allergic encephalomyelitis (EAE) using syngenic spinal cord homogenate in Freund's adjuvant on day 0 and 7. Mice exhibit relapsing-remitting episodes of paralysis and spasticity developed typically after 2-3 relapses, at about 80-100 days postinduction. Spasticity is assessed by measuring the force required for hindlimb flexion against a strain gauge prior to and following the administration of vehicle or test compound. Spasticity resulting from accumulating neurological deficit is associated with limb stiffness and is measured during remission from active paralytic attacks, where limbs lack functional movement and exhibit weak resistance to flexion. There is significant variation between the degree of spasticity between individual limbs and animals, which makes direct comparison between different groups difficult. Therefore, the forces from individual limbs are assessed, pairwise, using analysis of variance tests. Test compounds or vehicle are administered as appropriate during the experiment.

### Inflammation

### Experimental Colitis

Experimental colitis tests are models for inflammatory bowel diseases. The dextran sulfate sodium (DSS) experimental colitis model can be conducted as described in Kimball, E. S. et al Am. J. Physiol. Gastrointest. Liver Physiol. 291: G364-G371, 2006. Mice are provided with a solution of tap water containing 5% DSS (45 kDa) ad libitum over a 7-day period. The DSS solution is replenished daily, and the amount consumed is measured. At the end of this 7-day period, animals are euthanized, and their colons are examined for signs of inflammation and diarrhea. The colon length is measured from the oral end of the cecum to the anus. These measurements and observations are assigned a score as previously reported (Kimball, E. S. et al Am. J. Physiol. Gastrointest. Liver Physiol. 288:G1266-G1273, 2005). The sum of these three individual macroscopic indices are combined into a macroscopic score for each colon, where 0=normal and 11=maximally affected. Test compounds or vehicle are administered as appropriate during the experiment.

### Carrageenan Paw Inflammation

The carrageenan-induced inflammation of the paw can be used to test the anti-inflammatory properties of compounds. This model can be conducted as described in Holt, S. et al British Journal of Pharmacology 2005 146:467-476. Briefly, the animals are weighed and thereafter anaesthetised by intraperitoneal (i.p.) injection of pentobarbital (60 mg kg⁻¹). Acute inflammation is induced by i.pl. injection of 20 μl of carrageenan (2% w v⁻¹ in saline) into the right hind paw. Control animals receive a corresponding i.pl. injection of vehicle. The paw volume of the injected paw as well as the contralateral paw is then measured by a plethysmometer, before and 2 and 4 h after the carrageenan injection. At the 2 h time point, most of the animals have recovered from the anaesthesia, and at the 4 h time point all animals have recovered. The volume of the contralateral paw is subtracted from the volume of the injected paw, to obtain the oedema volume. Test compounds or vehicle are administered at least 15 minutes prior to carrageenan treatment.

### Glaucoma

### High Intraocular Pressure Ischemia

The high intraocular pressure (IOP) ischemia model is a model of acute glaucoma. It can be conducted as described in Nucci, C. et al Investigative Ophthalmology and Visual Science. 2007 48:2997-3004. Before induction of ischemia, animals are anesthetized with chloral hydrate (400 mg/kg i.p.). Corneal analgesia is achieved by using topical drops of 0.4% oxybuprocaine. Pupillary dilation is maintained using 0.5% tropicamide. The anterior chamber of the right eye is cannulated with a 27-gauge infusion needle connected to a 500-mL plastic container of sterile saline, then IOP is raised to 120 mm Hg for 45 minutes by elevating the saline reservoir. Retinal ischemia is confirmed by observing a whitening of the iris and loss of the red reflex of the retina. A sham procedure is performed without the elevation of the bottle in the contralateral eye. Rats sustaining an ischemic insult in the right eye and a sham procedure in the contralateral eye are divided into groups and treated with either vehicle or test compounds. Alternatively, rats are treated with vehicle or test compounds before the ischemic injury. After reperfusion, the animals are anesthetized (chloral hydrate, 400 mg/kg i.p.) and perfused through the left ventricle of the heart with 50 mL of heparinized PBS (pH 7.4), followed by 50 mL of 4% paraformaldehyde in PBS. Two hours after the perfusion-fixation procedure has been completed, the eyes are enucleated and postfixed in 4% paraformaldehyde for 72 hours. Serial coronal sections, cut along the vertical meridian of the eye passing through the optic nerve head, are stained with hematoxylin and eosin. The number of cells in the RGC layer is counted in six areas (25 μm² each) of each section (n=5 per eye), at a distance of 300 μm from the optic nerve head on the superior and inferior hemisphere, under a light microscope (40× magnification).

### Intraocular Pressure Test

The effect of test compounds on intraocular pressure (IOP) can be measured as described in Laine, K. et al Investigative Ophthalmology and Visual Science. 2002 43:3216-3222. For each experiment, rabbits are placed in plastic restraining boxes located in a quiet room. A drop of test solution containing either vehicle or test compound is instilled unilaterally into the left eye on the upper corneoscleral limbus. IOP is measured by using a pneumatonometer for pigmented rabbits or a handheld tonometer for albino rabbits. Before each measurement, 1 or 2 drops of topical anesthetic (0.04% oxybuprocaine) is applied to reduce discomfort. For every determination, at least two readings are taken from each treated (ipsilateral) and untreated (contralateral) eye, and the mean of these readings are used. IOP is measured at 1 hour before administration, then at 0, 0.5, 1, 2, 3, 4, and 5 hours after application of the eye drops. IOP at the time of administration of the eye drops (0 hour) is used as a baseline value.

### Appetite Enhancement

### Feeding Test

Test compounds can be assayed for appetite stimulation in a food consumption test as described for mice in Wiley, J. L. et al Br. J. Pharmacol. 2005 145: 293-300. Briefly, all compounds are tested in adult male ICR mice (25-32 g). For the feeding experiments, each ICR mouse is tested with each dose of a single test compound, presented in randomized order. The weight of food pellets is measured at 0.01 mg accuracy. At 24 h before the start of a feeding trial, all food is removed from the home cages of mice to be tested. The next day mice are transported to the laboratory at least 1 h before the beginning of the feeding trial. They are injected with the test compound or vehicle at the specified pre-session injection interval. Subsequently, they are placed in a clear plastic cage with thick brown paper lining the bottom and allowed access to a pre-measured amount of their regular lab chow. At the end of 1 h, mice are removed from the test cage and placed back into their home cage. The amount of food left in the test cage, including crumbs, is measured, and the amount consumed is calculated.

### Atherosclerosis

E3L transgenic mice express a mutated form of human ApoE3 which make them a suitable model for the testing of compounds with potential anti-atherosclerotic properties. The experiments can be conducted as described in de Haan, W. et al Atherosclerosis 2008 197:57-63. Briefly, male E3L mice are fed a semi-synthetic diet containing 15% (w/w) fat, supplemented with 0.25% (w/w) cholesterol in the presence or absence of test compounds for 8 weeks. Experiments are performed after 4 h of fasting. Plasma is obtained via tail vein bleeding and assayed for total cholesterol (TC) and for the distribution of lipids over plasma lipoproteins. Livers are isolated from control-treated and compound-treated mice after cervical dislocation and lipids extracted from them. The levels of total cholesterol, free cholesterol, cholesteryl esters and phospholipids are then determined.

### Activity of Exemplary Compounds of the Invention

The following compounds have been or can be prepared according to the methods of the invention. An enzymatic (fluorescence based) assay is performed as described above. The % Inhibition data for some of the representative compounds are given in Table 1 below. In Table 1, activity of each compound is expressed as follows:
++++ % Inhibition >75%
+++ % Inhibition >50%
++ % Inhibition >25%
+ % Inhibition <25%

TABLE 1
% Inhibition Data for Exemplary Compounds of the Invention

| COMPOUND | | MW | MW | % Inhibition |
|---|---|---|---|---|
| NO. | STRUCTURE | (Calcd) | (Obs) | @ 1 uM |
| 1 | 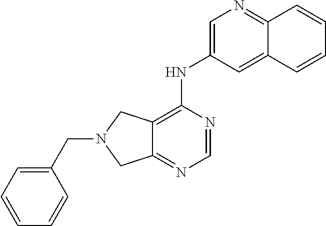 | 353.43 | 354.2 | ++++ |
| 2 | 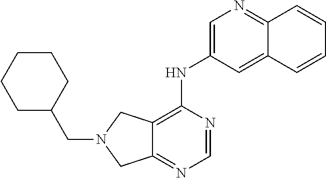 | 359.47 | 360.4 | ++++ |
| 3 | 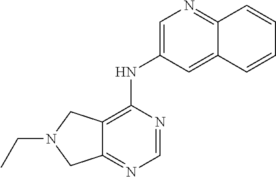 | 291.36 | | + |
| 4 | 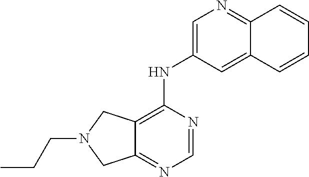 | 305.39 | | + |
| 5 | 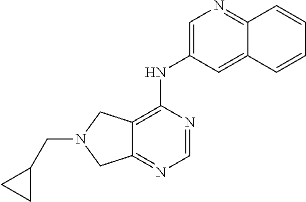 | 317.40 | | + |
| 6 | 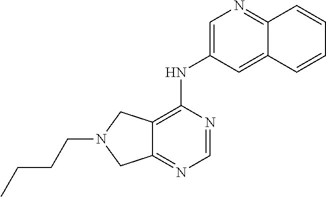 | 319.41 | | ++ |
| 7 | 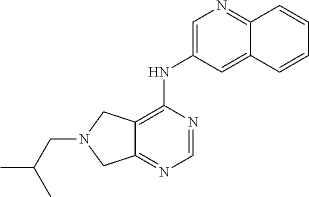 | 319.41 | | ++ |
| 8 | 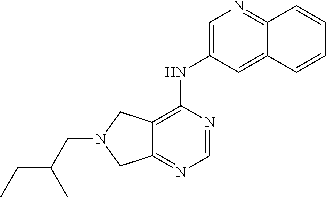 | 333.44 | | ++++ |
| 9 | 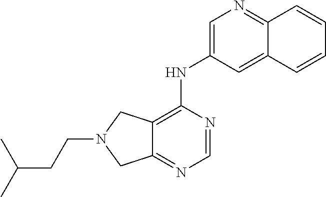 | 333.44 | 334.5 | +++ |
| 10 | 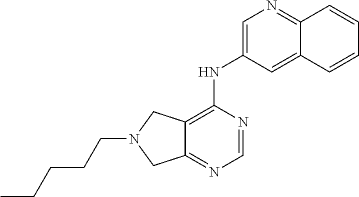 | 333.44 | 334.5 | +++ |
| 11 | 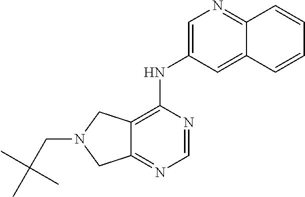 | 333.44 | 334.5 | ++ |
| 12 | 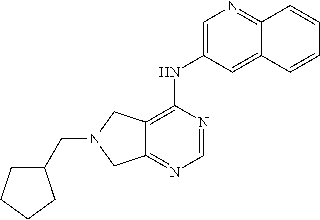 | 345.45 | 346.3 | ++++ |
| 13 | 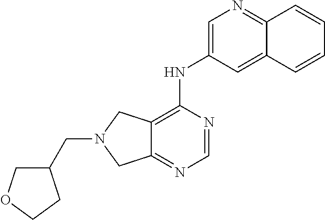 | 347.42 | 348.2 | ++ |
| 14 | 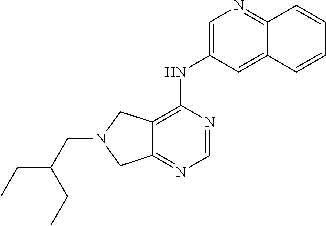 | 347.46 | 348.2 | ++++ |
| 15 | 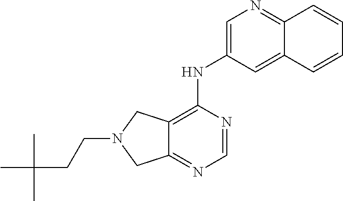 | 347.46 | 348.3 | ++ |
| 16 | 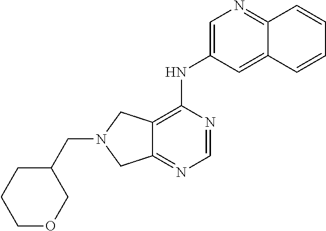 | 361.45 | 362.4 | +++ |
| 17 | 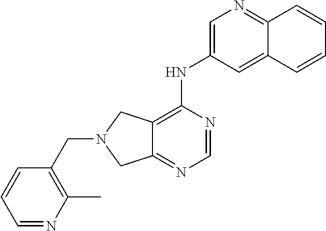 | 368.44 | 369.3 | ++++ |
| 18 | 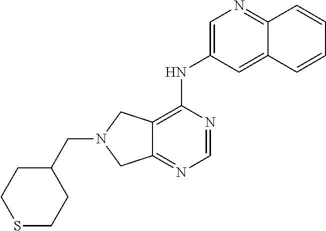 | 377.51 | 378.2 | ++++ |
| 19 | 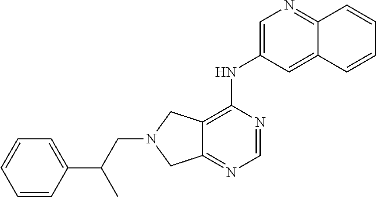 | 381.48 | 382.2 | ++++ |
| 20 | 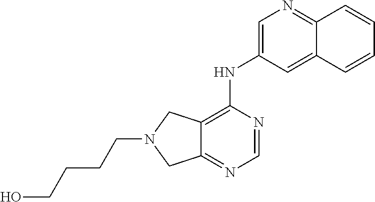 | 335.41 | 336.4 | + |
| 21 | 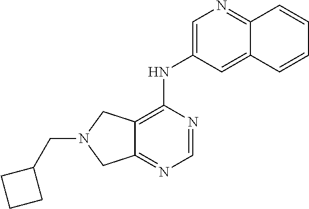 | 331.42 | 332.4 | +++ |
| 22 | 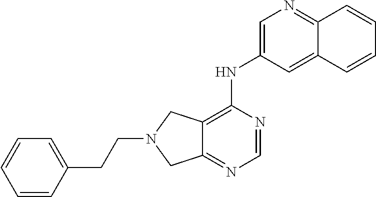 | 367.45 | 368.3 | ++++ |
| 23 | 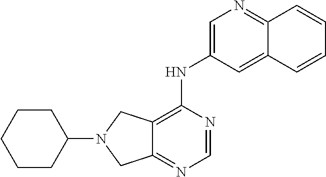 | 345.45 | 346.3 | ++ |
| 24 | 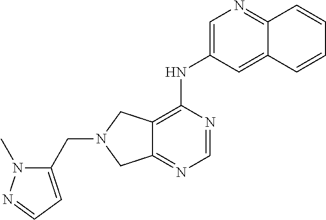 | 357.42 | 358.3 | ++ |
| 25 | 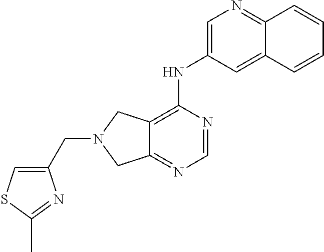 | 374.47 | 375.2 | + |
| 26 | 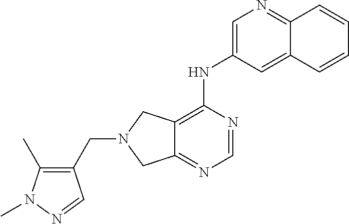 | 371.45 | 372.3 | ++ |
| 27 | 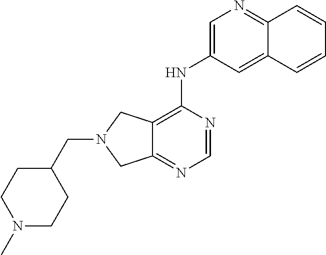 | 374.49 | 375.5 | + |
| 28 | 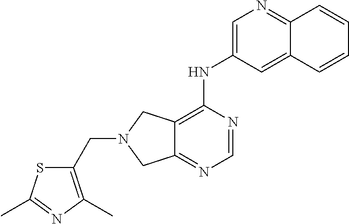 | 388.50 | 389.4 | ++++ |
| 29 | 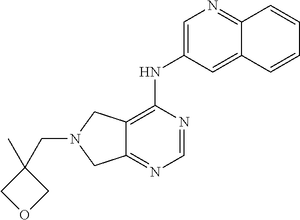 | 347.42 | 348.2 | ++ |
| 30 | 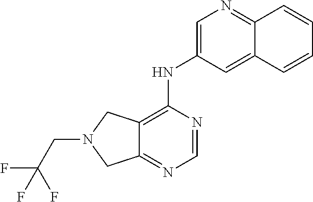 | 345.33 | 346.2 | + |
| 31 | 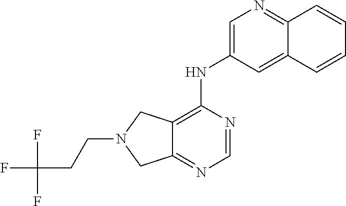 | 359.35 | 360.4 | +++ |
| 32 | 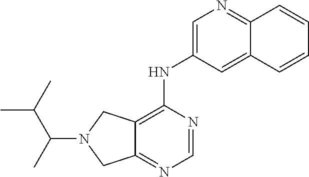 | 333.44 | 334.5 | ++ |
| 33 | 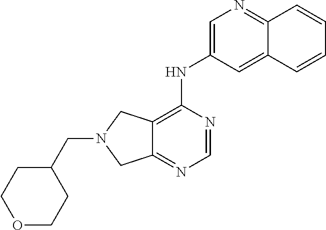 | 361.45 | 362.4 | ++++ |
| 34 | 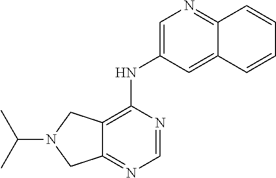 | 305.38 | 306.2 | + |
| 35 | 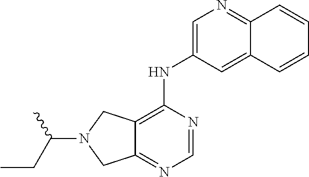 | 319.41 | 320.4 | + |
| 36 | 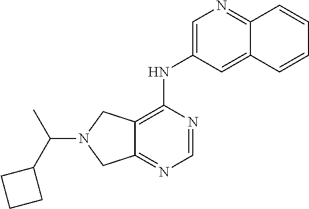 | 345.45 | 346.3 | ++ |
| 37 | 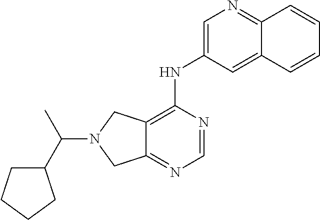 | 359.47 | 360.4 | +++ |
| 38 | 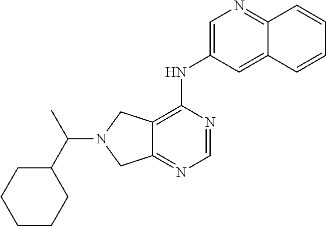 | 373.50 | 374.4 | ++++ |
| 39 | 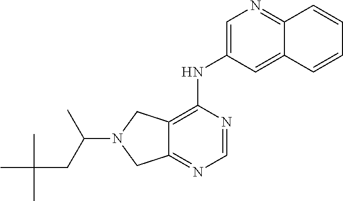 | 361.49 | 362.4 | + |
| 40 | 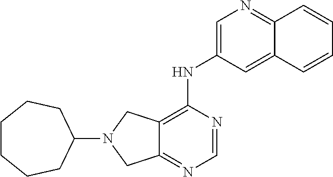 | 359.47 | 360.4 | ++ |
| 41 | 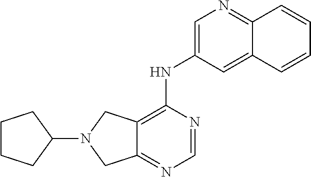 | 331.42 | 332.4 | +++ |
| 42 | 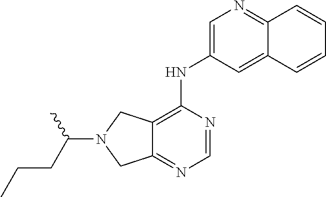 | 333.44 | 334 | ++ |
| 43 | 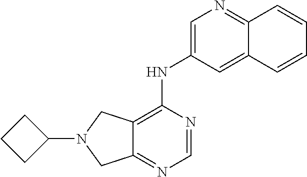 | 317.39 | 318.3 | + |
| 44 | 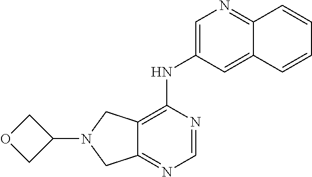 | 319.37 | 320.3 | + |
| 45 | 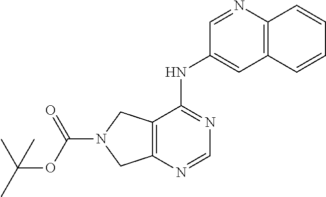 | 363.42 | 364.4 | +++ |
| 46 | 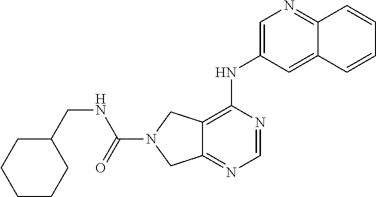 | 402.50 | 403.2 | + |
| 47 | 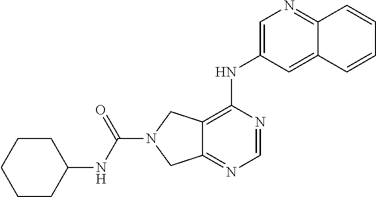 | 388.47 | 389.4 | + |
| 48 | 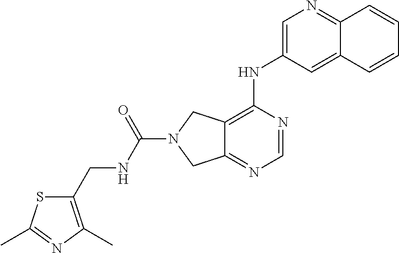 | 431.52 | 432.3 | + |
| 49 | 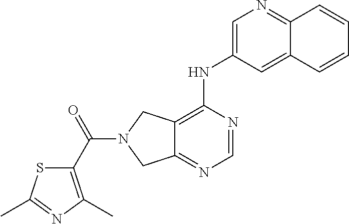 | 402.48 | 403.2 | +++ |
| 50 | 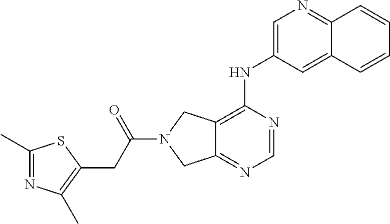 | 416.51 | 417.4 | + |
| 51 | 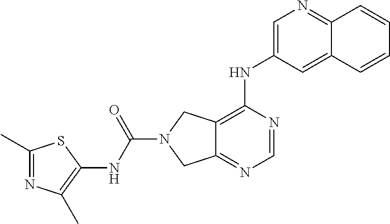 | 417.50 | 418.5 | + |
| 52 | 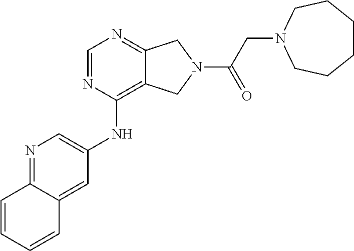 | 402.50 | 403.2 | + |
| 53 | 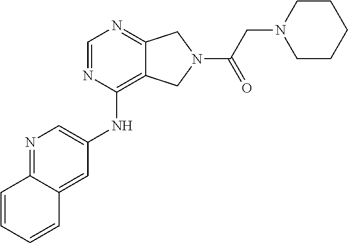 | 388.47 | 389.4 | + |
| 54 | 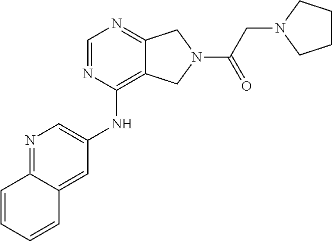 | 374.45 | 375.2 | + |
| 55 | 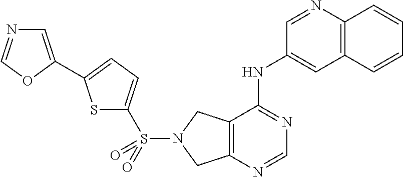 | 476.54 | 477.2 | ++ |
| 56 | 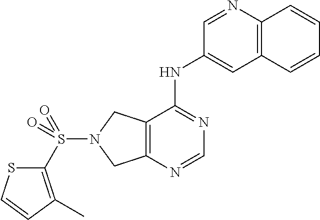 | 423.52 | 424.4 | ++ |
| 57 | 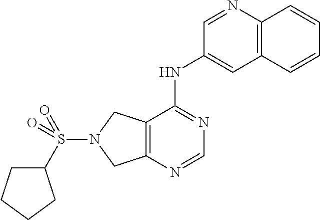 | 395.48 | 396.4 | ++ |
| 58 | 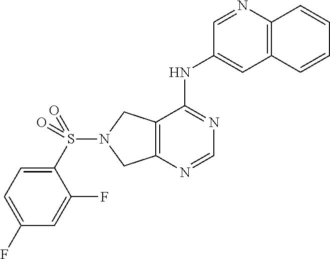 | 439.44 | 440.6 | ++ |
| 59 | 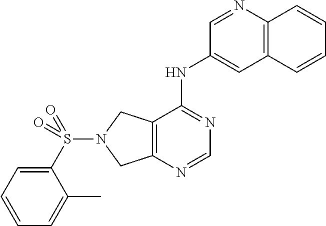 | 417.49 | 418.5 | ++++ |
| 60 | 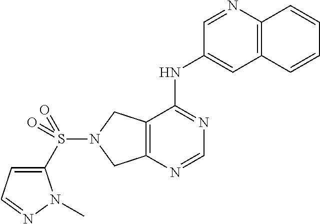 | 407.46 | 408 | +++ |
| 61 | 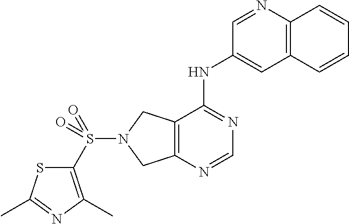 | 438.53 | 438.7 | +++ |
| 62 | 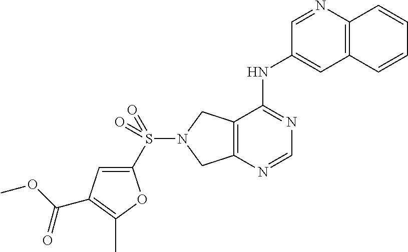 | 465.49 | 466 | ++ |
| 63 | 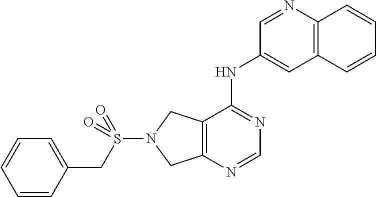 | 417.49 | 418 | + |
| 64 | 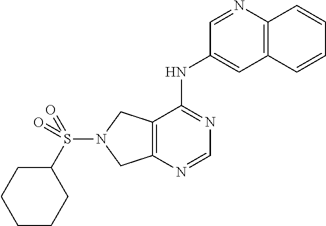 | 409.51 | 409.8 | +++ |
| 65 | 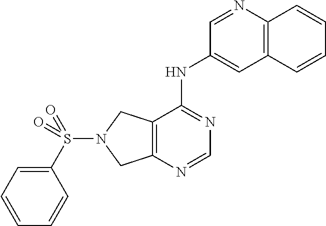 | 403.46 | 404.1 | ++ |
| 66 | 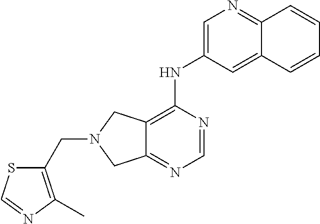 | 374.47 | 375.1 | ++++ |
| 67 | 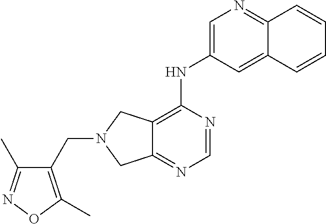 | 372.43 | 373.2 | ++ |
| 68 | 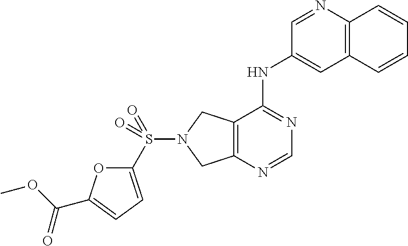 | 451.46 | 452.2 | ++++ |
| 69 | 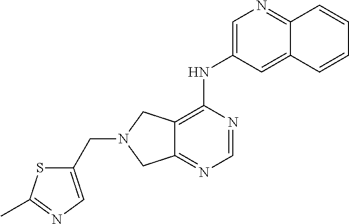 | 374.47 | 375.1 | +++ |
| 70 | 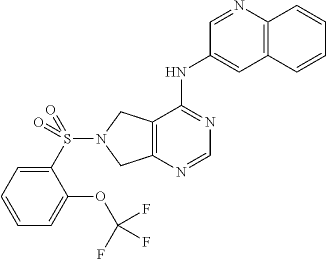 | 487.46 | 487.9 | ++ |
| 71 | 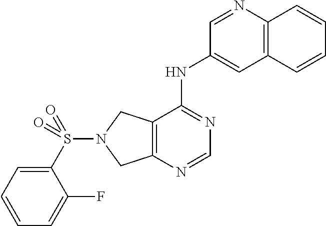 | 421.45 | 421.9 | ++ |
| 72 | 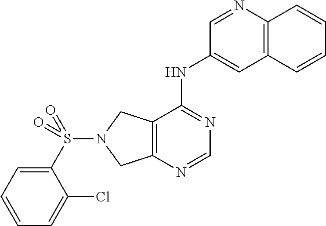 | 437.91 | 438.2 | +++ |
| 73 | 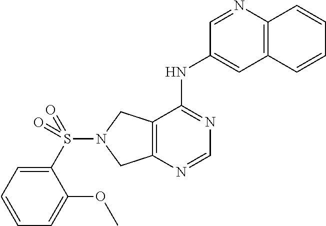 | 433.49 | 433.7 | ++ |
| 74 | 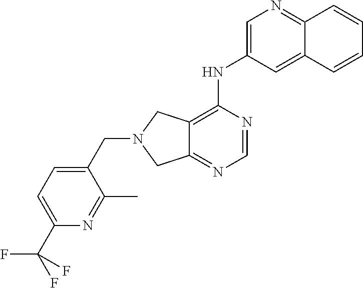 | 436.44 | 437.3 | ++ |
| 75 | 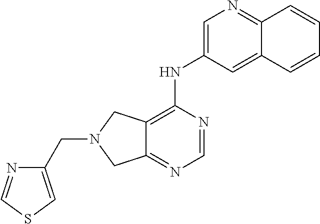 | 360.44 | 361.1 | ++ |
| 76 | 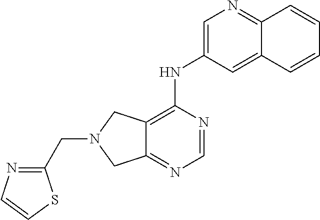 | 360.44 | 361.3 | ++ |
| 77 | 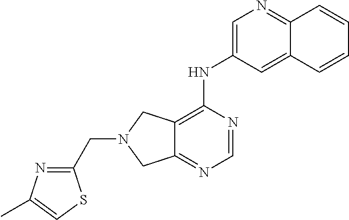 | 374.47 | 375.1 | +++ |
| 78 | 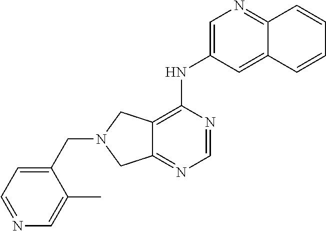 | 368.44 | 369.1 | ++++ |
| 79 | 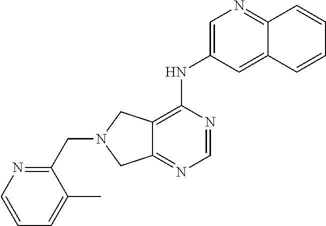 | 368.44 | 369.2 | + |
| 80 | 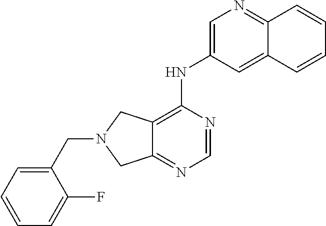 | 371.42 | 372.2 | ++++ |
| 81 | 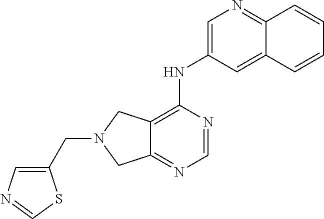 | 360.44 | 361.1 | ++ |
| 82 | 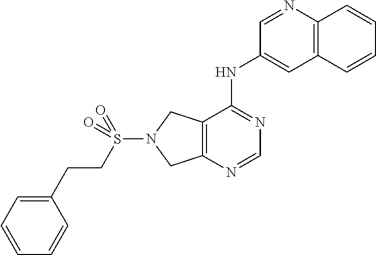 | 431.52 | 432.2 | + |
| 83 | 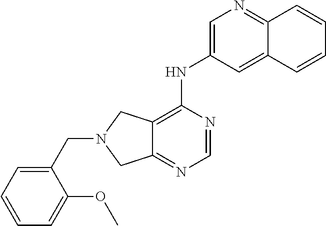 | 383.45 | 384.2 | ++++ |
| 84 | 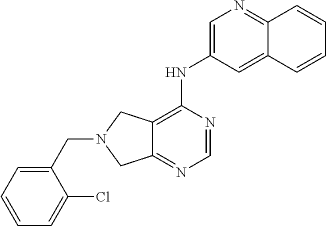 | 387.87 | 388.3 | ++++ |
| 85 | 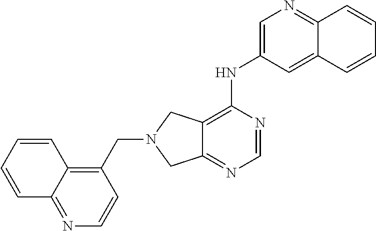 | 404.48 | 405.2 | +++ |
| 86 | 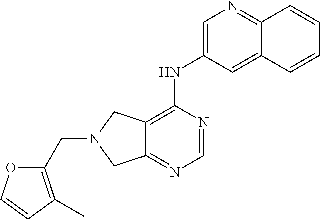 | 357.42 | 358.3 | ++++ |
| 87 | 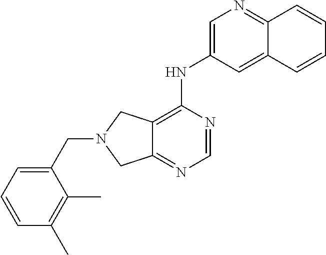 | 381.48 | 382.2 | ++++ |
| 88 | 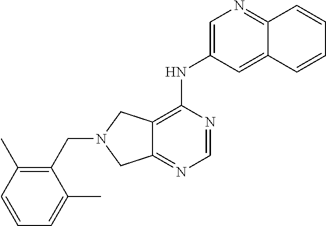 | 381.48 | 382.2 | +++ |
| 89 | 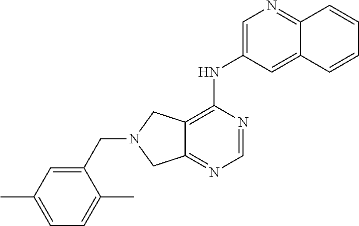 | 381.48 | 382.3 | ++++ |
| 90 | 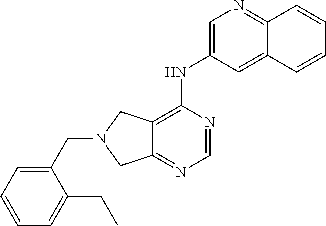 | 381.48 | 382 | ++++ |
| 91 | 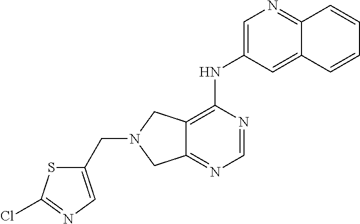 | 394.89 | 395.4 | +++ |
| 92 | 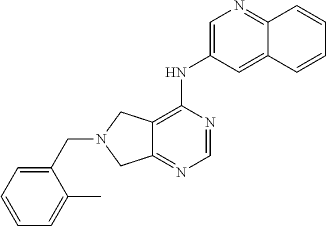 | 367.45 | 368.3 | ++++ |
| 93 | 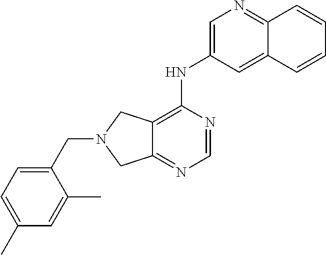 | 381.48 | 382.2 | ++ |
| 94 | 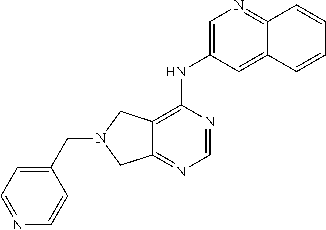 | 354.42 | 355.3 | ++++ |
| 95 | 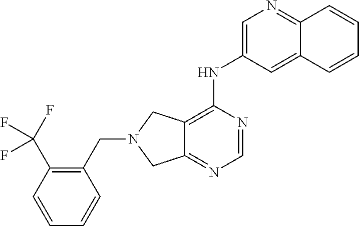 | 421.42 | 422.2 | ++++ |
| 96 | 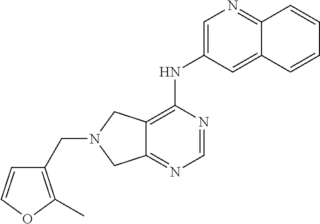 | 357.42 | 358.2 | +++ |
| 97 | 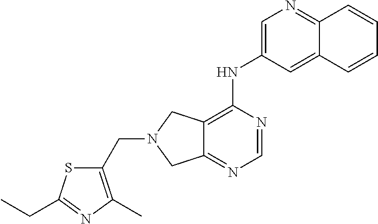 | 402.52 | 403 | ++++ |
| 98 | 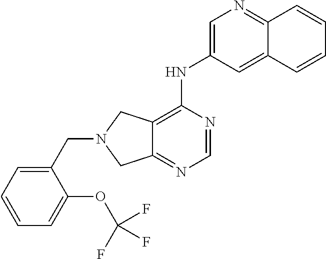 | 437.42 | 438.1 | ++++ |
| 99 | 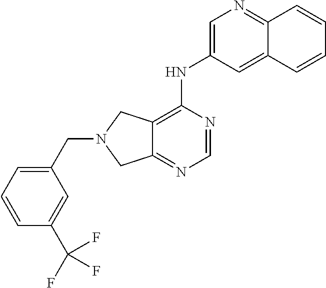 | 421.42 | 422.3 | ++++ |
| 100 | 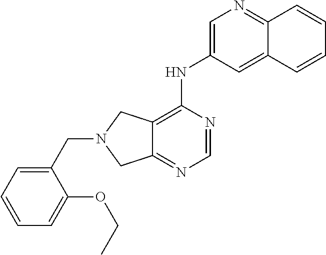 | 397.48 | 398.2 | ++++ |
| 101 | 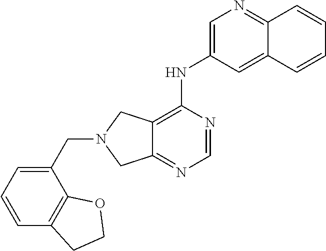 | 395.46 | 396.2 | ++++ |
| 102 | 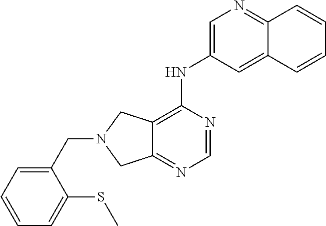 | 399.52 | 400.1 | ++++ |
| 103 | 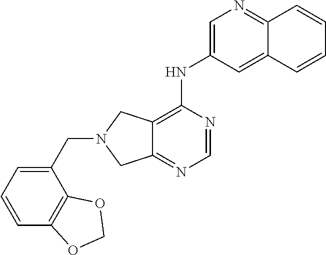 | 397.44 | 398.3 | ++++ |
| 104 | 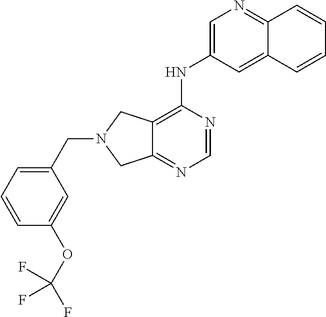 | 437.42 | 438.1 | ++++ |
| 105 | 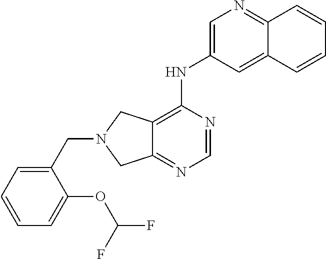 | 419.43 | 420.2 | ++++ |
| 106 | 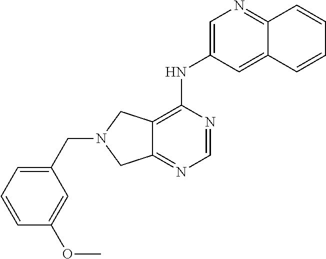 | 383.45 | 384.3 | ++++ |
| 107 | 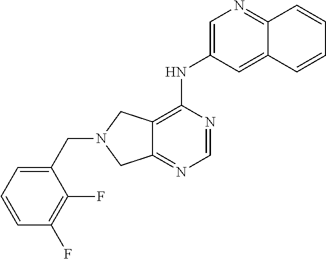 | 389.41 | 390.4 | ++++ |
| 108 | 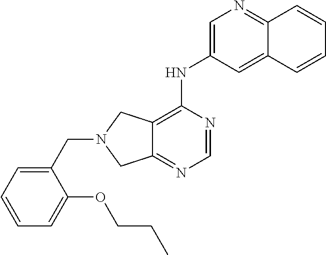 | 411.51 | 412.3 | +++ |
| 109 | 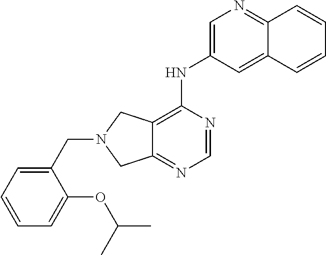 | 411.51 | 412.3 | +++ |
| 110 | 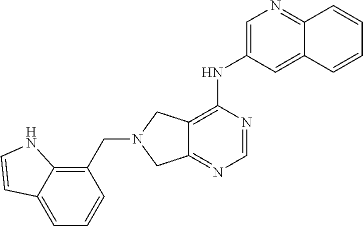 | 392.46 | 393.2 | ++++ |
| 111 | 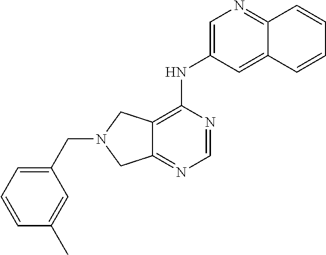 | 367.45 | 368.4 | ++++ |
| 112 | 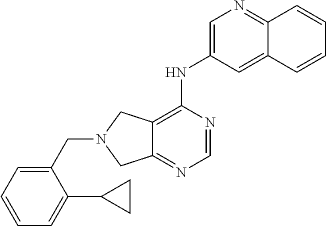 | 393.49 | 394.3 | ++++ |

The NMR data of exemplary compounds of the invention is provided in Table 2.

TABLE 2
NMR Data for Exemplary Compounds of the Invention

| ID | NMR (δ) |
|---|---|
| 28 | ¹H NMR (DMSO-d6) 9.35 (1H, s), 9.07 (1H, d), 8.81 (1H, d), 8.61 (1H, s), 7.95-7.89 |
| | (2H, m), 7.63-7.56 (2H, m), 4.04 (2H, s), 3.95 (4H, s), 2.58 (3H, s), 2.33 (3H, s). |
| 45 | ¹H NMR (400 MHz; DMSO-d6) 9.57 (s, 1H), 9.14-9.13 (m, 1H), 8.86-8.83 (m, 1H), |
| | 8.66 (d, J = 3.8 Hz, 1H), 7.97-7.92 (m, 2H), 7.66-7.56 (m, 2H), 4.66 (d, J = 11.1 Hz, |
| | 2H), 4.52 (d, J = 10.5 Hz, 2H), 1.51 (d, J = 9.1 Hz, 9H). |
| 55 | ¹H NMR (DMSO-d6) 9.57 (1H, s), 9.07 (1H, d), 8.79 (1H, d), 8.63 (1H, s), 8.52 (1H, |
| | s), 7.95 (1H, d), 7.92 (1H, dd), 7.88 (1H, d), 7.78 (1H, s), 7.64-7.62 (2H, m), 7.58 |
| | (1H, dd), 4.76 (2H, s), 4.62 (2H, s). |
| 56 | ¹H NMR (DMSO-d6) 9.58 (1H, s), 9.08 (1H, d), 8.81 (1H, d), 8.63 (1H, s), 7.96 (1H, |
| | d), 7.93 (1H, d) 7.66-7.62 (2H, m), 7.58 (1H, dd), 7.01 (1H, dd), 4.70 (2H, s), 4.54 |
| | (2H, s). |
| 57 | ¹H NMR (DMSO-d6) 9.55 (1H, s), 9.11 (1H, d), 8.85 (1H, d), 8.69 (1H, s), 7.98-7.93 |
| | (2H, m), 7.65 (1H, dt), 7.58 (1H, dt), 4.78 (2H, s), 4.62 (2H, s), 3.93-3.89 (1H, m), |
| | 2.03-1.87 (4H, m), 1.71-1.66 (2H, m), 1.63-1.55 (2H, m). |
| 58 | ¹H NMR (DMSO-d6) 9.57 (1H, s), 9.08 (1H, d), 8.81 (1H, d), 8.65 (1H, s), 8.03-7.92 |
| | (3H, m), 7.66-7.55 (3H, m), 7.35 (1H, dt), 4.76 (2H, s), 4.59 (2H, s). |
| 59 | ¹H NMR (DMSO-d6) 9.54 (1H, s), 9.08 (1H, d), 8.82 (1H, d), 8.67 (1H, s), 7.97-7.94 |
| | (2H, m), 7.88 (1H, dd), 7.66-7.55 (3H, m), 7.51-7.45 (2H, m), 4.71 (2H, s), 4.58 |
| | (2H, s), 2.64 (3H, s). |
| 60 | ¹H NMR (DMSO-d6) 9.56 (1H, s), 9.08 (1H, d), 8.81 (1H, d), 8.67 (1H, s), 7.98-7.92 |
| | (2H, m), 7.66-7.57 (3H, m), 6.94 (1H, d), 4.78 (2H, s), 4.65 (2H, s), 4.12 (3H, s). |
| 61 | ¹H NMR (DMSO-d6) 9.59 (2H, s), 9.09 (1H, t), 8.81 (1H, dd), 8.65 (1H, s), 7.98-7.92 |
| | (2H, m), 7.64 (1H, dt), 7.58 (1H, dt), 4.73 (2H, s), 4.56 (2H, s), 2.66 (6H, s). |
| 62 | ¹H NMR (DMSO-d6) 9.56 (1H, s), 9.09 (1H, d), 8.82 (1H, d), 8.66 (1H, s), 8.97-8.92 |
| | (2H, m), 7.65 (1H, dd), 1.57 (1H, dd), 7.43 (1H, s), 4.75 (2H, s), 4.60 (2H, s), 3.77 |
| | (3H, s), 2.58 (3H, s). |
| 63 | ¹H NMR (DMSO-d6) 9.47 (1H, s), 9.09 (1H, d), 8.84 (1H, d), 8.69 (1H, s), 7.95 (2H, |
| | dt), 7.65 (1H, dt), 7.58 (1H, dt), 7.40-7.36 (2H, m), 7.34-7.31 (3H, m), 4.67 (2H, s), |
| | 4.56 (4H, s). |
| 65 | ¹H NMR (DMSO-d6) 9.51 (1H, s), 9.06 (1H, d), 8.78 (1H, d), 8.60 (1H, s), 7.97-7.90 |
| | (4H, m), 7.72-7.55 (5H, m), 4.68 (2H, s), 4.56 (2H, s). |
| 66 | ¹H NMR (DMSO-d6) 9.36 (1H, s), 9.07 (1H, d), 8.97 (1H, s), 8.81 (1H, d), 8.61 (1H, |
| | s), 7.95-7.89 (2H, m), 7.64-7.54 (2H, m), 4.12 (2H, s), 3.97 (4H, d), 2.43 (3H, s). |
| 67 | ¹H NMR (DMSO-d6) 9.33 (1H, s), 9.07 (1H, d), 8.79 (1H, d), 8.60 (1H, s), 7.95-7.90 |
| | (2H, m), 7.65-7.55 (2H, m), 3.87 (4H, d), 3.72 (2H, s), 2.42 (3H, s), 2.44 (3H, s). |
| 68 | ¹H NMR (DMSO-d6) 9.61 (1H, s), 9.09 (1H, d), 8.22 (1H, s), 8.65 (1H, s), 7.97-7.92 |
| | (2H, m), 7.66-7.56 (2H, m), 7.47 (2H, s), 4.79 (2H, s), 4.61 (2H, s), 3.81 (3H, s). |
| 69 | ¹H NMR (DMSO-d6) 9.36 (1H, s), 9.07 (1H, d), 8.81 (1H, d), 8.60 (1H, s), 7.95-7.89 |
| | (2H, m), 7.64-7.54 (3H, m), 4.11 (2H, s), 3.95 (4H, s), 2.64 (3H, s). |
| 70 | ¹H NMR (DMSO-d6) 9.57 (1H, d), 9.08 (1H, d), 8.79 (1H, d), 8.65 (1H, s), 8.06 (1H, |
| | dd), 7.97-7.92 (2H, m), 7.84 (1H, dt), 7.66-7.55 (4H, m), 4.74 (2H, s), 4.64 (2H, s). |
| 71 | ¹H NMR (DMSO-d6) 9.57 (1H, s), 9.08 (1H, d), 8.81 (1H, d), 8.64 (1H, s), 7.97-7.91 |
| | (3H, m), 7.78-7.76 (1H, m), 7.65 (1H, dt), 7.57 (1H, dt), 7.52-7.44 (2H, m), 4.78 |
| | (2H, s), 4.60 (2H, s). |
| 72 | ¹H NMR (DMSO-d6) 9.55 (1H, s), 9.06 (1H, d), 8.80 (1H, d), 8.65 (1H, s), 8.06 (1H, |
| | dd), 7.96-7.91 (2H, m), 7.75-7.71 (2H, m), 7.63-7.56 (3H, m), 4.79 (2H, s), 4.66 |
| | (2H, s). |
| 73 | ¹H NMR (DMSO-d6) 9.53 (1H, s), 9.08 (1H, d), 8.82 (1H, d), 8.64 (1H, s), 7.96-7.88 |
| | (3H, m), 7.66-7.62 (2H, m), 7.59-7.57 (1H, m), 7.22 (1H, d), 7.15 (1H, dt), 4.74 |
| | (2H, s), 4.59 (2H, s), 3.79 (3H, s). |
| 74 | ¹H NMR (DMSO-d6) 9.33 (1H, s), 9.06 (1H, d), 8.80 (1H, d), 8.62 (1H, s), 8.08 (1H, |
| | d), 7.95-7.90 (2H, m), 7.77 (1H, s), 7.62-7.56 (2H, m), 4.08 (2H, s), 4.00 (2H, s), |
| | 3.96 (2H, s), 2.64 (3H, s). |
| 75 | ¹H NMR (DMSO-d6) 9.38 (1H, s), 9.11 (1H, d), 9.08 (1H, d), 8.81 (1H, d), 8.60 (1H, |
| | s), 7.96-7.90 (2H, m), 7.66-7.54 (3H, m), 4.13 (2H, s), 4.06 (2H, s), 4.01 (2H, s). |
| 76 | ¹H NMR (DMSO-d6) 9.38 (1H, s), 9.07 (1H, d), 8.82 (1H, d), 8.62 (1H, s), 7.96-7.90 |
| | (2H, m), 7.79 (1H, d), 7.74 (1H, d), 7.63-7.56 (3H, m), 4.33 (2H, s), 4.11 (2H, s), 4.08 |
| | (2H, s). |
| 77 | ¹H NMR (DMSO-d6) 9.38 (1H, s), 9.07 (1H, d), 8.82 (1H, d), 8.62 (1H, s), 7.96-7.90 |
| | (2H, m), 7.64-7.56 (2H, m), 7.26 (1H, s), 4.26 (2H, s), 4.09 (2H, s), 4.07 (2H, s), 2.36 |
| | (3H, s). |
| 78 | ¹H NMR (DMSO-d6) 9.34 (1H, s), 9.06 (1H, d), 8.80 (1H, d), 8.62 (1H, s), 8.42 (1H, |
| | d), 8.39 (1H, s), 7.95-7.92 (2H, m), 7.64-7.56 (2H, m), 7.44 (1H, d), 3.99-3.95 (6H, |
| | m), 2.34 (3H, s). |
| 79 | 1H (DMSO-d6) 9.34 (1H, s), 9.06 (1H, d), 8.79 (1H, d), 8.59 (1H, s), 8.38 (1H, dd), |
| | 7.95-7.89 (2H, m), 7.64-7.55 (3H, m), 7.25 (1H, dd), 4.07 (2H, s), 3.96 (4H, s), 2.42 |
| | (3H, s). |
| 81 | ¹H NMR (400 MHz, DMSO-d6) 9.37 (s, 1H), 9.10 (d, J = 0.7, 1H), 9.07 (d, J = 2.6, |
| | 1H), 8.81 (d, J = 2.4, 1H), 8.61 (s, 1H), 7.93-7.90 (m, 3H), 7.64-7.62 (dt, 1H), 7.56 |
| | (dt, 1H), 4.21 (s, 2H), 3.97 (s, 4H). |
| 83 | ¹H NMR (400 MHz, DMSO-d6) 9.34 (s, 1H), 9.07 (d, J = 2.5, 1H), 8.81 (d, J = 2.5, |
| | 1H), 8.60 (s, 1H), 7.95-7.90 (m, 2H), 7.62 (td, 1H), 7.57 (td, 1H), 7.44 (d, J = 5.9, |
| | 1H), 7.26 (m, 1H), 7.06 6.94 (m, 2H), 3.96-3.94 (m, 6H), 3.82 (s, 3H). |
| 86 | ¹H NMR (400 MHz, DMSO-d6) 9.37 (s, 1H), 9.08 (d, J = 2.6, 1H), 8.79 (d, J = 2.4, |
| | 1H), 8.58 (s, 1H), 7.98 7.87 (m, 2H), 7.67 7.51 (m, 3H), 6.34 (d, J = 1.8, 1H), 3.96 (s, |
| | 2H), 3.92 3.86 (m, J = 5.4, 4H), 2.06 (s, 3H). |
| 88 | ¹H NMR (400 MHz, DMSO-d6) 9.32 (s, 1H), 9.05 (d, J = 2.5, 1H), 8.78 (d, J = 2.3, |
| | 1H), 8.59 (s, 1H), 7.94 (d, J = 8.1, 1H), 7.90 (dd, J = 7.0, 1H), 7.66 7.59 (m, 1H), 7.59 |
| | 7.51 (m, 1H), 7.13 6.99 (m, 3H), 3.97 3.85 (m, 6H), 2.43 (s, 6H). |
| 91 | ¹H NMR (400 MHz, DMSO-d6) 9.36 (s, 1H), 9.07 (d, J = 2.6, 1H), 8.81 (d, J = 2.4, |
| | 1H), 8.61 (s, 1H), 7.95 (d, J = 8.3, 1H), 7.91 (dd, J = 8.2, 1.2, 1H), 7.70 (s, 1H), 7.67 |
| | 7.60 (m, 1H), 7.60 7.52 (m, 1H), 4.16 (s, 2H), 3.99 (s, 4H). |
| 95 | ¹H NMR (400 MHz, DMSO-d6) 9.33 (s, 1H), 9.06 (d, J = 2.6, 1H), 8.82 (d, J = 2.3, |
| | 1H), 8.62 (s, 1H), 7.98 7.87 (m, 3H), 7.80 7.70 (m, 2H), 7.65 7.49 (m, 3H), 4.14 (s, |
| | 2H), 4.01 (s, 2H), 3.97 (s, 2H). |
| 99 | ¹H NMR (400 MHz, MeOD) 9.06 (s, 1H), 8.88 (s, 1H), 8.61 (s, 1H), 7.99 (d, J = 8.3, |
| | 1H), 7.91 (d, J = 8.2, 1H), 7.81 (s, 1H), 7.76 (d, J = 7.3, 1H), 7.72 7.57 (m, 4H), 4.11 |
| | (s, 2H), 4.04 (s, 4H). |
| 104 | ¹H NMR (400 MHz, DMSO-d6) 9.35 (s, 1H), 9.06 (d, J = 2.6, 1H), 8.80 (d, J = 2.4, |
| | 1H), 8.61 (s, 1H), 7.94 (d, J = 8.3, 1H), 7.91 (dd, J = 8.2, 1.2, 1H), 7.67 7.45 (m, 4H), |
| | 7.41 (s, 1H), 7.35 7.25 (m, 1H), 4.02 (s, 2H), 3.96 (s, 2H), 3.94 (s, 2H). |

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

Chemical structures shown herein were prepared using ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

1. A compound having a formula: 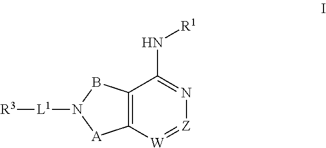
wherein
each A and B is independently CR^{2a}R^{2b};
W and Z are independently N or CR⁴;
L¹ is a single bond or substituted or unsubstituted C₁-C₅ alkylene, —CO—, —NHC(O)—, —OC(O)—, —SO—, or S(O)₂—;
R¹ is selected from a substituted or unsubstituted aryl or heteroaryl;
each of R^{2a}, and R^{2b} is independently selected from hydrogen, and substituted or unsubstituted C₁-C₆ alkyl;
R³ is selected from substituted or unsubstituted C₁-C₆alkyl, substituted or unsubstituted C₃-C₈cycloalkyl, substituted or unsubstituted heterocycloalkyl, substituted or unsubstituted aryl, and substituted or unsubstituted heteroaryl; and
R⁴ is independently selected from H, C₁-C₆alkyl, substituted C₁-C₆alkyl, acyl, substituted acyl, substituted or unsubstituted acylamino, substituted or unsubstituted amino, substituted or unsubstituted alkoxy, alkoxycarbonyl, substituted alkoxycarbonyl arylalkyloxy, substituted arylalkyloxy, amino, aryl, substituted aryl, arylalkyl, substituted arylalkyl, substituted sulfanyl, substituted sulfinyl, substituted sulfonyl, substituted or unsubstituted aminosulfonyl, sulfo, sulfonic acid ester, azido, carboxy, substituted or unsubstituted carbamoyl, cyano, substituted or unsubstituted C₃-C₈cycloalkyl, substituted or unsubstituted heterocycloalkyl, halo, heteroaryloxy, substituted or unsubstituted heteroaryl, hydroxy, nitro, and thiol;
or a pharmaceutically acceptable salt, solvate or prodrug thereof; and stereoisomers, isotopic variants and tautomers thereof;
provided that the compound is other than
N-[5-(1,1-dimethylethyl)-2-methylphenyl]-6,7-dihydro-2-[4-(4-methoxyphenyl)-1-piperazinyl]-6-(methylsulfonyl)-5H-pyrrolo[3,4-d]pyrimidin-4-amine;
4-[[5-(1,1-dimethylethyl)-2-methylphenyl]amino]-5,7-dihydro-2-[4-(4-methoxyphenyl)-1-piperazinyl]-6H-Pyrrolo[3,4-d]pyrimidine-6-carboxylic acid, 1,1-dimethylethyl ester;
2-chloro-4-[[3-(dimethylamino)-5-methoxyphenyl]amino]-5,7-dihydro-6H-pyrrolo[3,4-d]pyrimidine-6-carboxylic acid, 9H-fluoren-9-ylmethyl ester;
2-chloro-5,7-dihydro-4-[(5-methyl-1H-pyrazol-3-yl)amino]-6H-pyrrolo[3,4-d]pyrimidine-6-carboxylic acid, 9H-fluoren-9-ylmethyl ester; and
N-(2,3-dihydro-1H-inden-2-yl)-6,7-dihydro-2-(methylthio)-6-(phenylmethyl)-5H-pyrrolo[3,4-d]pyrimidin-4-amine.

2. (canceled)

3. (canceled)

4. A compound according to claim 1, wherein A is CHR^{2b} or CH₂; B is CHR^{2b} or CH₂; or wherein each of A and B is CH₂.

5. (canceled)

6. (canceled)

7. A compound according to claim 1, wherein R¹ is substituted or unsubstituted phenyl; or substituted or unsubstituted heteroaryl.

8. (canceled)

9. A compound according to claim 7, wherein R¹ is substituted or unsubstituted pyridyl, quinolinyl, isoquinolinyl, substituted or unsubstituted benzodioxole, substituted or unsubstituted benzodioxane, substituted or unsubstituted benzofuran, substituted or unsubstituted benzothiophene, and substituted or unsubstituted benzodioxepine.

10. A compound according to claim 9, wherein R¹ is substituted or unsubstituted quinoline-3-yl.

11. (canceled)

12. (canceled)

13. (canceled)

14. A compound according to claim 1, wherein L¹ is a single bond or substituted or unsubstituted C₁-C₅ alkylene.

15. (canceled)

16. A compound according to claim 1, wherein L¹ is —CH₂—, —C(Me)H—, —CH₂CH₂—, —C(Me)HCH₂—, or —CH₂C(Me)H—.

17. A compound according to claim 1, wherein L¹ is —CO—, —NHC(O)—, —OC(O)—, —SO—, or —S(O)₂—.

18. (canceled)

19. (canceled)

20. (canceled)

21. A compound according to claim 1 wherein W is N, and Z is CR⁴; and R⁴ is H, Me, CF₃, Cl or F.

22. (canceled)

23. A compound according to claim 1 wherein the compound is according to formula IIa, IIb, IIc, IId, IIe, IIf; IIg, IIh, IIi, or IIj: 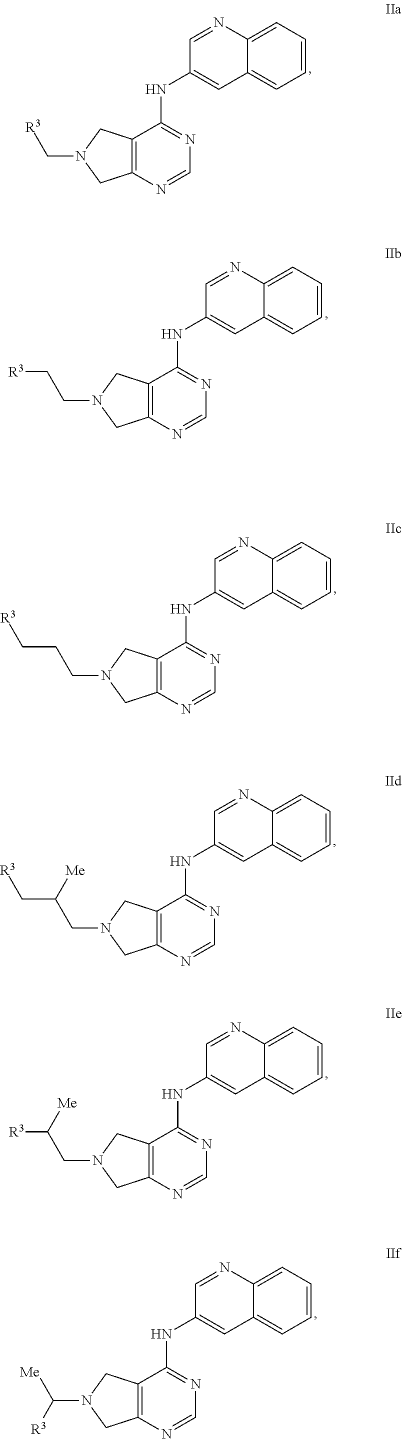 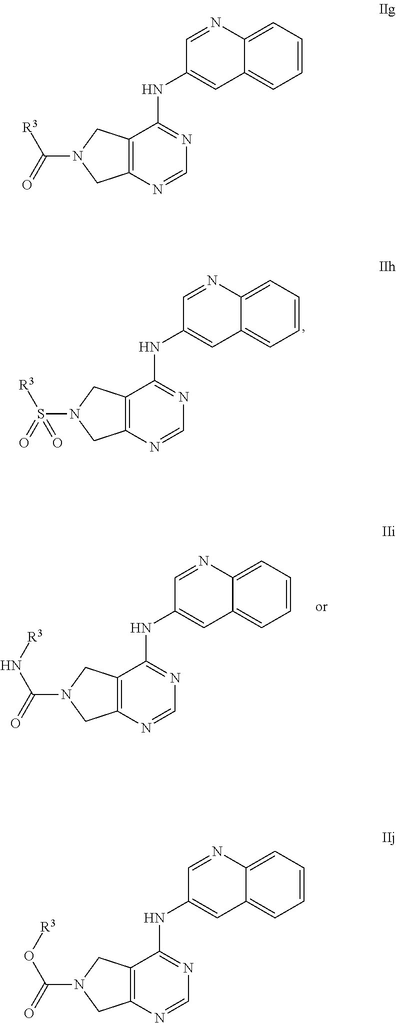
and wherein R³ is as in claim 1.

24. (canceled)

25. A compound according to claim 1 wherein R³ is C₁-C₆alkyl, substituted with Cl, F or OH.

26. A compound according to claim 1 wherein R³ is Me, Et, n-Pr, i-Pr, n-Bu, t-Bu, 2,2-dimethylpropyl, 2-methylpropyl, or CF₃.

27. A compound according to claim 1 wherein R³ is C₃-C₈cycloalkyl, unsubstituted or substituted with one or more substituents independently selected from halo, hydroxyl, C₁-C₆alkyl, alkoxy and haloalkyl.

28. (canceled)

29. (canceled)

30. (canceled)

31. A compound according to claim 1 wherein R³ is pyrrolidinyl, piperidinyl, morpholinyl, tetrahydropyranyl, or piperazinyl, unsubstituted or substituted with one or more substituents independently selected from halo, hydroxyl, C₁-C₆alkyl, alkoxy and haloC₁-C₆alkyl.

32. (canceled)

33. (canceled)

34. A compound according to claim 1 wherein R³ is phenyl or pyridyl, unsubstituted or substituted with one or more substituents independently selected from halo, hydroxyl, amino, cyano, sulfo, substituted sulfanyl, substituted sulfinyl, substituted sulfonyl, substituted or unsubstituted amido, substituted or unsubstituted amino, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

35. (canceled)

36. A compound according to claim 1 wherein R³ is pyrimidinyl, quinolinyl, isoquinolinyl, indolyl, indazolyl, benzofuranyl, benzothiophenyl, benzoxazinyl, benzodioxolanyl, pyrrolyl, furanyl, pyrazolyl, imidazolyl, triazolyl, oxazolyl, thienyl, thiazolyl, oxadiazolyl, or thiadiazolyl, unsubstituted or substituted with one or more substituents independently selected from halo, hydroxyl, amino, cyano, sulfo, substituted sulfonyl, substituted sulfanyl, amido, carboxy, C₁-C₆ alkoxycarbonyl, C₁-C₆alkyl, substituted C₁-C₆alkyl, alkenyl, substituted alkenyl, alkynyl, substituted alkynyl, and sulfonamide.

37. (canceled)

38. (canceled)

39. (canceled)

40. A compound according to claim 1 wherein the compound is according to formula IIIa, IIIb, IIIc, IIId, IIIe, IIIf, IIIg, IIIh or IIIi: 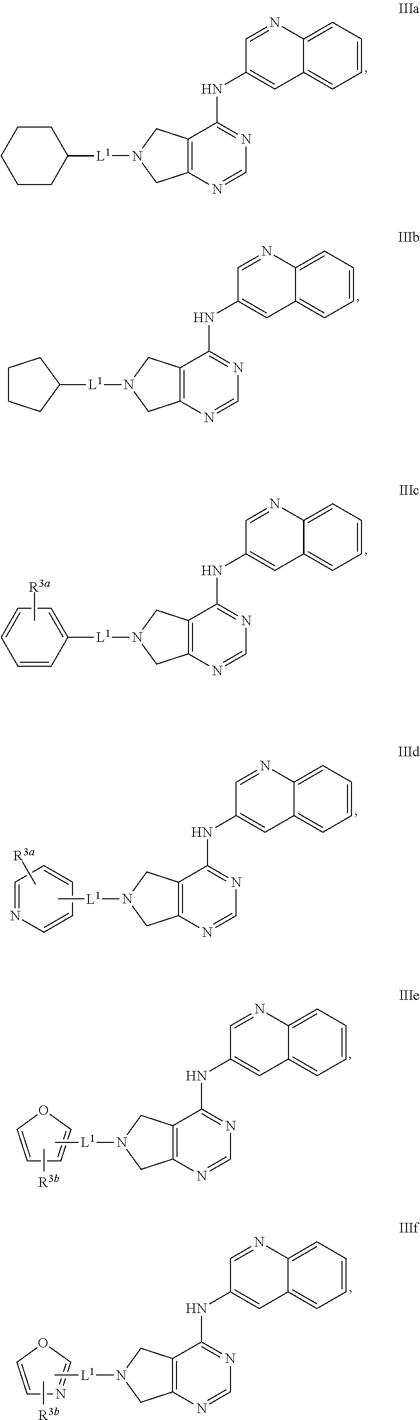 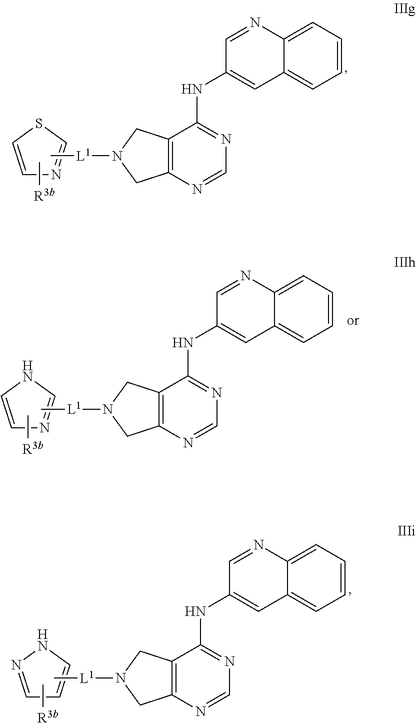
and wherein L¹ is a single bond, —CH₂—, —CO—, —NHC(O)—, —OC(O)—, or S(O)₂; and R^{3a} is H, alkyl, alkoxy, halo, haloalkyl, thioalkyl, or haloalkyloxy; and R^{3b} is H or alkyl.

41. (canceled)

42. (canceled)

43. (canceled)

44. A compound according to claim 1 wherein the compound is according to formula IVa, IVb, IVc, IVd, IVe, IVf, IVg, IVh or IVi: 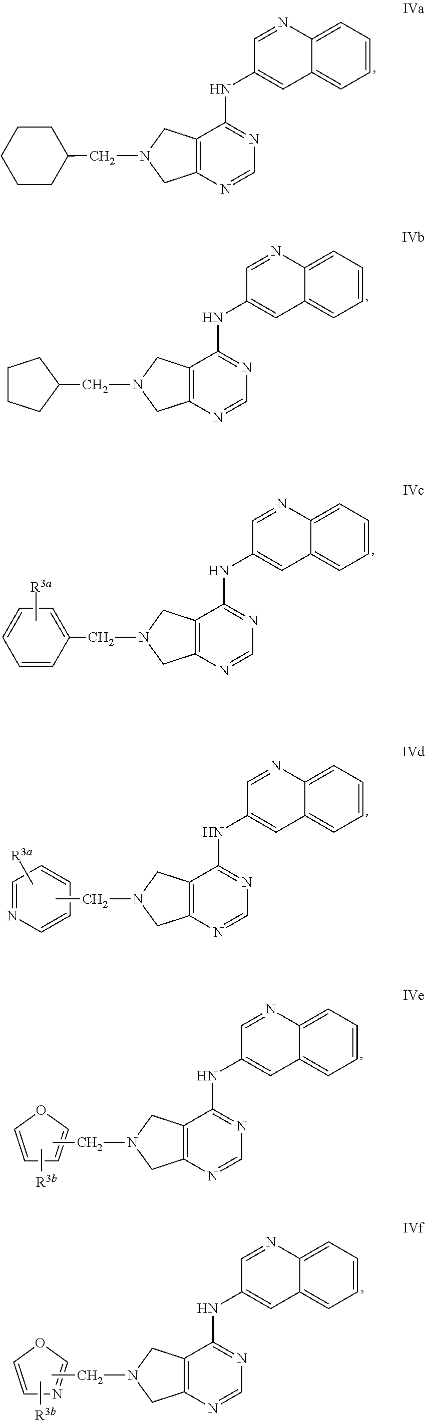 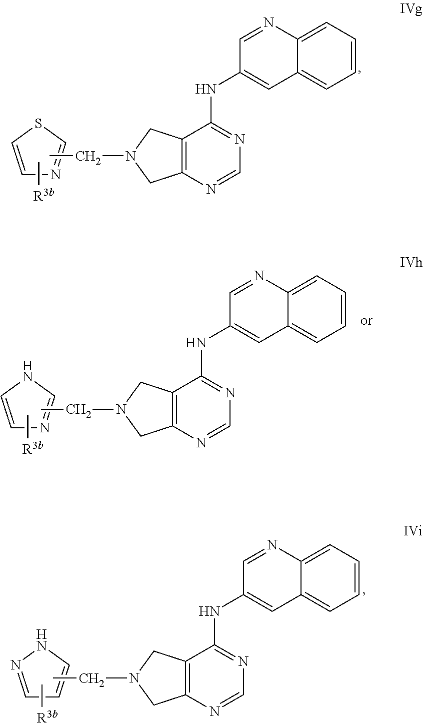
and wherein R^{3a} is H, alkyl, alkoxy, halo, haloalkyl, thioalkyl, or haloalkyloxy; and R^{3b} is H or alkyl.

45. A compound according to claim 1 wherein the compound is according to formula Va, Vb, Vc, Vd, Ve, Vf, Vg, Vh or Vi: 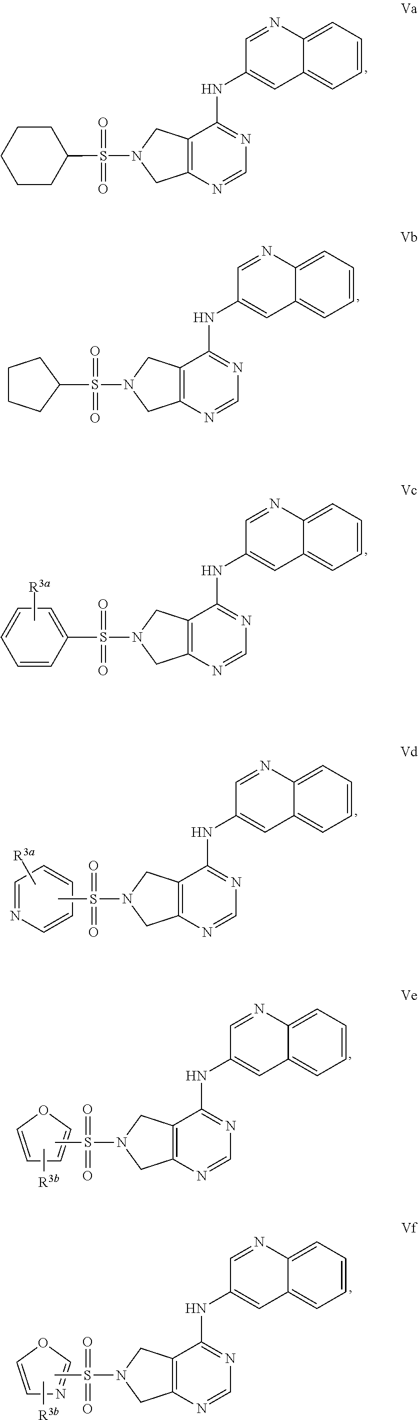 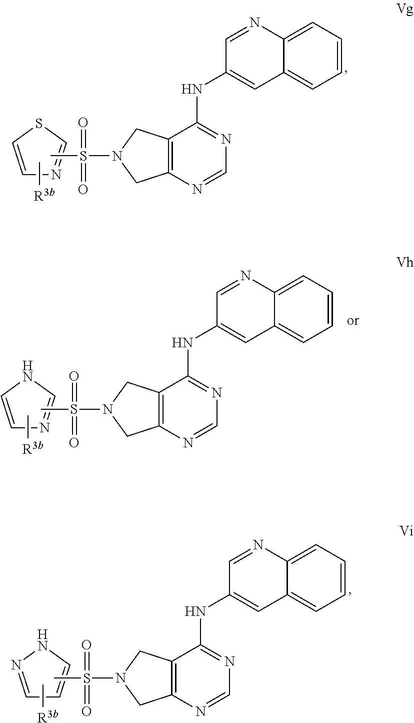
and wherein R^{3a} is H, alkyl, alkoxy, halo, haloalkyl, thioalkyl, or haloalkyloxy; and R^{3b} is H or alkyl.

46. (canceled)

47. (canceled)

48. (canceled)

49. (canceled)

50. A compound according to claim 1, wherein the compound is selected from:
(6-Benzyl-6,7-dihydro-5H-pyrrolo[3,4-d]pyrimidin-4-yl)-quinolin-3-yl-amine;
(6-Cyclohexylmethyl-6,7-dihydro-5H-pyrrolo[3,4-d]pyrimidin-4-yl)-quinolin-3-yl-amine;
(6-Cyclopentylmethyl-6,7-dihydro-5H-pyrrolo[3,4-d]pyrimidin-4-yl)-quinolin-3-yl-amine;
[6-(2-Ethyl-butyl)-6,7-dihydro-5H-pyrrolo[3,4-d]pyrimidin-4-yl]-quinolin-3-yl-amine;
[6-(2-Methyl-pyridin-3-ylmethyl)-6,7-dihydro-5H-pyrrolo[3,4-d]pyrimidin-4-yl]-quinolin-3-yl-amine;
Quinolin-3-yl-[6-(tetrahydro-thiopyran-4-ylmethyl)-6,7-dihydro-5H-pyrrolo[3,4-d]pyrimidin-4-yl]-amine;
[6-(2-Phenyl-propyl)-6,7-dihydro-5H-pyrrolo[3,4-d]pyrimidin-4-yl]-quinolin-3-yl-amine;
(6-Phenethyl-6,7-dihydro-5H-pyrrolo[3,4-d]pyrimidin-4-yl)-quinolin-3-yl-amine;
[6-(2,4-Dimethyl-thiazol-5-ylmethyl)-6,7-dihydro-5H-pyrrolo[3,4-d]pyrimidin-4-yl]-quinolin-3-yl-amine;
Quinolin-3-yl-[6-(tetrahydro-pyran-4-ylmethyl)-6,7-dihydro-5H-pyrrolo[3,4-d]pyrimidin-4-yl]-amine;
[6-(1-Cyclohexyl-ethyl)-6,7-dihydro-5H-pyrrolo[3,4-d]pyrimidin-4-yl]-quinolin-3-yl-amine;
Quinolin-3-yl-[6-(toluene-2-sulfonyl)-6,7-dihydro-5H-pyrrolo[3,4-d]pyrimidin-4-yl]-amine;
[6-(4-Methyl-thiazol-5-ylmethyl)-6,7-dihydro-5H-pyrrolo[3,4-d]pyrimidin-4-yl]-quinolin-3-yl-amine;
5-[4-(Quinolin-3-ylamino)-5,7-dihydro-pyrrolo[3,4-d]pyrimidine-6-sulfonyl]-furan-2-carboxylic acid methyl ester;
[6-(3-Methyl-pyridin-4-ylmethyl)-6,7-dihydro-5H-pyrrolo[3,4-d]pyrimidin-4-yl]-quinolin-3-yl-amine;
[6-(2-Fluoro-benzyl)-6,7-dihydro-5H-pyrrolo[3,4-d]pyrimidin-4-yl]-quinolin-3-yl-amine;
[6-(2-Methoxy-benzyl)-6,7-dihydro-5H-pyrrolo[3,4-d]pyrimidin-4-yl]-quinolin-3-yl-amine;
[6-(2-Chloro-benzyl)-6,7-dihydro-5H-pyrrolo[3,4-d]pyrimidin-4-yl]-quinolin-3-yl-amine;
[6-(3-Methyl-furan-2-ylmethyl)-6,7-dihydro-5H-pyrrolo[3,4-d]pyrimidin-4-yl]-quinolin-3-yl-amine;
[6-(2,3-Dimethyl-benzyl)-6,7-dihydro-5H-pyrrolo[3,4-d]pyrimidin-4-yl]-quinolin-3-yl-amine;
[6-(2,5-Dimethyl-benzyl)-6,7-dihydro-5H-pyrrolo[3,4-d]pyrimidin-4-yl]-quinolin-3-yl-amine;
[6-(2-Ethyl-benzyl)-6,7-dihydro-5H-pyrrolo[3,4-d]pyrimidin-4-yl]-quinolin-3-yl-amine;
[6-(2-Methyl-benzyl)-6,7-dihydro-5H-pyrrolo[3,4-d]pyrimidin-4-yl]-quinolin-3-yl-amine;
(6-Pyridin-4-ylmethyl-6,7-dihydro-5H-pyrrolo[3,4-d]pyrimidin-4-yl)-quinolin-3-yl-amine;
Quinolin-3-yl-[6-(2-trifluoromethyl-benzyl)-6,7-dihydro-5H-pyrrolo[3,4-d]pyrimidin-4-yl]-amine;
[6-(2-Ethyl-4-methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzyl)-6,7-dihydro-5H-pyrrolo[3,4-d]pyrimidin-4-yl]-amine;
Quinolin-3-yl-[6-(3-trifluoromethyl-benzyl)-6,7-dihydro-5H-pyrrolo[3,4-d]pyrimidin-4-yl]-amine;
[6-(2-Ethoxy-benzyl)-6,7-dihydro-5H-pyrrolo[3,4-d]pyrimidin-4-yl]-quinolin-3-yl-amine;
[6-(2,3-Dihydro-benzofuran-7-ylmethyl)-6,7-dihydro-5H-pyrrolo[3,4-d]pyrimidin-4-yl]-quinolin-3-yl-amine;
[6-(2-Methylsulfanyl-benzyl)-6,7-dihydro-5H-pyrrolo[3,4-d]pyrimidin-4-yl]-quinolin-3-yl-amine;
(6-Benzo[1,3]dioxol-4-ylmethyl-6,7-dihydro-5H-pyrrolo[3,4-d]pyrimidin-4-yl)-quinolin-3-yl-amine;
Quinolin-3-yl-[6-(3-trifluoromethoxy-benzyl)-6,7-dihydro-5H-pyrrolo[3,4-d]pyrimidin-4-yl]-amine;
[6-(2-Difluoromethoxy-benzyl)-6,7-dihydro-5H-pyrrolo[3,4-d]pyrimidin-4-yl]-quinolin-3-yl-amine;
(6-Benzyl-6,7-dihydro-5H-pyrrolo[3,4-d]pyrimidin-4-yl)-quinolin-3-yl-amine;
(6-Cyclohexylmethyl-6,7-dihydro-5H-pyrrolo[3,4-d]pyrimidin-4-yl)-quinolin-3-yl-amine;
(6-Ethyl-6,7-dihydro-5H-pyrrolo[3,4-d]pyrimidin-4-yl)-quinolin-3-yl-amine;
(6-Propyl-6,7-dihydro-5H-pyrrolo[3,4-d]pyrimidin-4-yl)-quinolin-3-yl-amine;
(6-Cyclopropylmethyl-6,7-dihydro-5H-pyrrolo[3,4-d]pyrimidin-4-yl)-quinolin-3-yl-amine;
(6-Butyl-6,7-dihydro-5H-pyrrolo[3,4-d]pyrimidin-4-yl)-quinolin-3-yl-amine;
(6-Isobutyl-6,7-dihydro-5H-pyrrolo[3,4-d]pyrimidin-4-yl)-quinolin-3-yl-amine;
[6-(2-Methyl-butyl)-6,7-dihydro-5H-pyrrolo[3,4-d]pyrimidin-4-yl]-quinolin-3-yl-amine;
[6-(3-Methyl-butyl)-6,7-dihydro-5H-pyrrolo[3,4-d]pyrimidin-4-yl]-quinolin-3-yl-amine;
(6-Pentyl-6,7-dihydro-5H-pyrrolo[3,4-d]pyrimidin-4-yl)-quinolin-3-yl-amine;
[6-(2,2-Dimethyl-propyl)-6,7-dihydro-5H-pyrrolo[3,4-d]pyrimidin-4-yl]-quinolin-3-yl-amine;
(6-Cyclopentylmethyl-6,7-dihydro-5H-pyrrolo[3,4-d]pyrimidin-4-yl)-quinolin-3-yl-amine;
Quinolin-3-yl-[6-(tetrahydro-furan-3-ylmethyl)-6,7-dihydro-5H-pyrrolo[3,4-d]pyrimidin-4-yl]-amine;
[6-(2-Ethyl-butyl)-6,7-dihydro-5H-pyrrolo[3,4-d]pyrimidin-4-yl]-quinolin-3-yl-amine;
[6-(3,3-Dimethyl-butyl)-6,7-dihydro-5H-pyrrolo[3,4-d]pyrimidin-4-yl]-quinolin-3-yl-amine;
Quinolin-3-yl-[6-(tetrahydro-pyran-3-ylmethyl)-6,7-dihydro-5H-pyrrolo[3,4-d]pyrimidin-4-yl]-amine;
[6-(2-Methyl-pyridin-3-ylmethyl)-6,7-dihydro-5H-pyrrolo[3,4-d]pyrimidin-4-yl]-quinolin-3-yl-amine;
Quinolin-3-yl-[6-(tetrahydro-thiopyran-4-ylmethyl)-6,7-dihydro-5H-pyrrolo[3,4-d]pyrimidin-4-yl]-amine;
[6-(2-Phenyl-propyl)-6,7-dihydro-5H-pyrrolo[3,4-d]pyrimidin-4-yl]-quinolin-3-yl-amine;
4-[4-(Quinolin-3-ylamino)-5,7-dihydro-pyrrolo[3,4-d]pyrimidin-6-yl]-butan-1-ol;
(6-Cyclobutylmethyl-6,7-dihydro-5H-pyrrolo[3,4-d]pyrimidin-4-yl)-quinolin-3-yl-amine;
(6-Phenethyl-6,7-dihydro-5H-pyrrolo[3,4-d]pyrimidin-4-yl)-quinolin-3-yl-amine;
(6-Cyclohexyl-6,7-dihydro-5H-pyrrolo[3,4-d]pyrimidin-4-yl)-quinolin-3-yl-amine;
[6-(2-Methyl-2H-pyrazol-3-ylmethyl)-6,7-dihydro-5H-pyrrolo[3,4-d]pyrimidin-4-yl]-quinolin-3-yl-amine;
[6-(2-Methyl-thiazol-4-ylmethyl)-6,7-dihydro-5H-pyrrolo[3,4-d]pyrimidin-4-yl]-quinolin-3-yl-amine;
[6-(1,5-Dimethyl-1H-pyrazol-4-ylmethyl)-6,7-dihydro-5H-pyrrolo[3,4-d]pyrimidin-4-yl]-quinolin-3-yl-amine;
[6-(1-Methyl-piperidin-4-ylmethyl)-6,7-dihydro-5H-pyrrolo[3,4-d]pyrimidin-4-yl]-quinolin-3-yl-amine;
[6-(2,4-Dimethyl-thiazol-5-ylmethyl)-6,7-dihydro-5H-pyrrolo[3,4-d]pyrimidin-4-yl]-quinolin-3-yl-amine;
[6-(3-Methyl-oxetan-3-ylmethyl)-6,7-dihydro-5H-pyrrolo[3,4-d]pyrimidin-4-yl]-quinolin-3-yl-amine;
Quinolin-3-yl-[6-(2,2,2-trifluoro-ethyl)-6,7-dihydro-5H-pyrrolo[3,4-d]pyrimidin-4-yl]-amine;
Quinolin-3-yl-[6-(3,3,3-trifluoro-propyl)-6,7-dihydro-5H-pyrrolo[3,4-d]pyrimidin-4-yl]-amine;
[6-(1,2-Dimethyl-propyl)-6,7-dihydro-5H-pyrrolo[3,4-d]pyrimidin-4-yl]-quinolin-3-yl-amine;
Quinolin-3-yl-[6-(tetrahydro-pyran-4-ylmethyl)-6,7-dihydro-5H-pyrrolo[3,4-d]pyrimidin-4-yl]-amine;
(6-Isopropyl-6,7-dihydro-5H-pyrrolo[3,4-d]pyrimidin-4-yl)-quinolin-3-yl-amine;
(6-sec-Butyl-6,7-dihydro-5H-pyrrolo[3,4-d]pyrimidin-4-yl)-quinolin-3-yl-amine;
[6-(1-Cyclobutyl-ethyl)-6,7-dihydro-5H-pyrrolo[3,4-d]pyrimidin-4-yl]-quinolin-3-yl-amine;
[6-(1-Cyclopentyl-ethyl)-6,7-dihydro-5H-pyrrolo[3,4-d]pyrimidin-4-yl]-quinolin-3-yl-amine;
[6-(1-Cyclohexyl-ethyl)-6,7-dihydro-5H-pyrrolo[3,4-d]pyrimidin-4-yl]-quinolin-3-yl-amine;
Quinolin-3-yl-[6-(1,3,3-trimethyl-butyl)-6,7-dihydro-5H-pyrrolo[3,4-d]pyrimidin-4-yl]-amine;
(6-Cycloheptyl-6,7-dihydro-5H-pyrrolo[3,4-d]pyrimidin-4-yl)-quinolin-3-yl-amine;
(6-Cyclopentyl-6,7-dihydro-5H-pyrrolo[3,4-d]pyrimidin-4-yl)-quinolin-3-yl-amine;
[6-(1-Methyl-butyl)-6,7-dihydro-5H-pyrrolo[3,4-d]pyrimidin-4-yl]-quinolin-3-yl-amine;
(6-Cyclobutyl-6,7-dihydro-5H-pyrrolo[3,4-d]pyrimidin-4-yl)-quinolin-3-yl-amine;
(6-Oxetan-3-yl-6,7-dihydro-5H-pyrrolo[3,4-d]pyrimidin-4-yl)-quinolin-3-yl-amine;
4-(Quinolin-3-ylamino)-5,7-dihydro-pyrrolo[3,4-d]pyrimidine-6-carboxylic acid tert-butyl ester;
4-(Quinolin-3-ylamino)-5,7-dihydro-pyrrolo[3,4-d]pyrimidine-6-carboxylic acid cyclohexylmethyl-amide;
4-(Quinolin-3-ylamino)-5,7-dihydro-pyrrolo[3,4-d]pyrimidine-6-carboxylic acid cyclohexylamide;
4-(Quinolin-3-ylamino)-5,7-dihydro-pyrrolo[3,4-d]pyrimidine-6-carboxylic acid (2,4-dimethyl-thiazol-5-ylmethyl)-amide;
(2,4-Dimethyl-thiazol-5-yl)-[4-(quinolin-3-ylamino)-5,7-dihydro-pyrrolo[3,4-d]pyrimidin-6-yl]-methanone;
2-(2,4-Dimethyl-thiazol-5-yl)-1-[4-(quinolin-3-ylamino)-5,7-dihydro-pyrrolo[3,4-d]pyrimidin-6-yl]-ethanone;
4-(Quinolin-3-ylamino)-5,7-dihydro-pyrrolo[3,4-d]pyrimidine-6-carboxylic acid (2,4-dimethyl-thiazol-5-yl)-amide;
2-Azepan-1-yl-1-[4-(quinolin-3-ylamino)-5,7-dihydro-pyrrolo[3,4-d]pyrimidin-6-yl]-ethanone;
2-Piperidin-1-yl-1-[4-(quinolin-3-ylamino)-5,7-dihydro-pyrrolo[3,4-d]pyrimidin-6-yl]-ethanone;
2-Pyrrolidin-1-yl-1-[4-(quinolin-3-ylamino)-5,7-dihydro-pyrrolo[3,4-d]pyrimidin-6-yl]-ethanone;
[6-(5-Oxazol-5-yl-thiophene-2-sulfonyl)-6,7-dihydro-5H-pyrrolo[3,4-d]pyrimidin-4-yl]-quinolin-3-yl-amine;
[6-(3-Methyl-thiophene-2-sulfonyl)-6,7-dihydro-5H-pyrrolo[3,4-d]pyrimidin-4-yl]-quinolin-3-yl-amine;
(6-Cyclopentanesulfonyl-6,7-dihydro-5H-pyrrolo[3,4-d]pyrimidin-4-yl)-quinolin-3-yl-amine;
[6-(2,4-Difluoro-benzenesulfonyl)-6,7-dihydro-5H-pyrrolo[3,4-d]pyrimidin-4-yl]-quinolin-3-yl-amine;
Quinolin-3-yl-[6-(toluene-2-sulfonyl)-6,7-dihydro-5H-pyrrolo[3,4-d]pyrimidin-4-yl]-amine;
[6-(2-Methyl-2H-pyrazole-3-sulfonyl)-6,7-dihydro-5H-pyrrolo[3,4-d]pyrimidin-4-yl]-quinolin-3-yl-amine;
[6-(2,4-Dimethyl-thiazole-5-sulfonyl)-6,7-dihydro-5H-pyrrolo[3,4-d]pyrimidin-4-yl]-quinolin-3-yl-amine;
2-Methyl-5-[4-(quinolin-3-ylamino)-5,7-dihydro-pyrrolo[3,4-d]pyrimidine-6-sulfonyl]-furan-3-carboxylic acid methyl ester;
(6-Phenylmethanesulfonyl-6,7-dihydro-5H-pyrrolo[3,4-d]pyrimidin-4-yl)-quinolin-3-yl-amine;
(6-Cyclohexanesulfonyl-6,7-dihydro-5H-pyrrolo[3,4-d]pyrimidin-4-yl)-quinolin-3-yl-amine;
(6-Benzenesulfonyl-6,7-dihydro-5H-pyrrolo[3,4-d]pyrimidin-4-yl)-quinolin-3-yl-amine;
[6-(4-Methyl-thiazol-5-ylmethyl)-6,7-dihydro-5H-pyrrolo[3,4-d]pyrimidin-4-yl]-quinolin-3-yl-amine;
[6-(3,5-Dimethyl-isoxazol-4-ylmethyl)-6,7-dihydro-5H-pyrrolo[3,4-d]pyrimidin-4-yl]-quinolin-3-yl-amine;
5-[4-(Quinolin-3-ylamino)-5,7-dihydro-pyrrolo[3,4-d]pyrimidine-6-sulfonyl]-furan-2-carboxylic acid methyl ester;
[6-(2-Methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzenesulfonyl)-6,7-dihydro-5H-pyrrolo[3,4-d]pyrimidin-4-yl]-amine;
[6-(2-Fluoro-benzenesulfonyl)-6,7-dihydro-5H-pyrrolo[3,4-d]pyrimidin-4-yl]-quinolin-3-yl-amine;
[6-(2-Chloro-benzenesulfonyl)-6,7-dihydro-5H-pyrrolo[3,4-d]pyrimidin-4-yl]-quinolin-3-yl-amine;
[6-(2-Methoxy-benzenesulfonyl)-6,7-dihydro-5H-pyrrolo[3,4-d]pyrimidin-4-yl]-quinolin-3-yl-amine;
[6-(2-Methyl-6-trifluoromethyl-pyridin-3-ylmethyl)-6,7-dihydro-5H-pyrrolo[3,4-d]pyrimidin-4-yl]-quinolin-3-yl-amine;
Quinolin-3-yl-(6-thiazol-4-ylmethyl-6,7-dihydro-5H-pyrrolo[3,4-d]pyrimidin-4-yl)-amine;
Quinolin-3-yl-(6-thiazol-2-ylmethyl-6,7-dihydro-5H-pyrrolo[3,4-d]pyrimidin-4-yl)-amine;
[6-(4-Methyl-thiazol-2-ylmethyl)-6,7-dihydro-5H-pyrrolo[3,4-d]pyrimidin-4-yl]-quinolin-3-yl-amine;
[6-(3-Methyl-pyridin-4-ylmethyl)-6,7-dihydro-5H-pyrrolo[3,4-d]pyrimidin-4-yl]-quinolin-3-yl-amine;
[6-(3-Methyl-pyridin-2-ylmethyl)-6,7-dihydro-5H-pyrrolo[3,4-d]pyrimidin-4-yl]-quinolin-3-yl-amine;
[6-(2-Fluoro-benzyl)-6,7-dihydro-5H-pyrrolo[3,4-d]pyrimidin-4-yl]-quinolin-3-yl-amine;
Quinolin-3-yl-(6-thiazol-5-ylmethyl-6,7-dihydro-5H-pyrrolo[3,4-d]pyrimidin-4-yl)-amine;
[6-(2-Phenyl-ethanesulfonyl)-6,7-dihydro-5H-pyrrolo[3,4-d]pyrimidin-4-yl]-quinolin-3-yl-amine;
[6-(2-Methoxy-benzyl)-6,7-dihydro-5H-pyrrolo[3,4-d]pyrimidin-4-yl]-quinolin-3-yl-amine;
[6-(2-Chloro-benzyl)-6,7-dihydro-5H-pyrrolo[3,4-d]pyrimidin-4-yl]-quinolin-3-yl-amine;
Quinolin-3-yl-(6-quinolin-4-ylmethyl-6,7-dihydro-5H-pyrrolo[3,4-d]pyrimidin-4-yl)-amine;
[6-(3-Methyl-furan-2-ylmethyl)-6,7-dihydro-5H-pyrrolo[3,4-d]pyrimidin-4-yl]-quinolin-3-yl-amine;
[6-(2,3-Dimethyl-benzyl)-6,7-dihydro-5H-pyrrolo[3,4-d]pyrimidin-4-yl]-quinolin-3-yl-amine;
[6-(2,6-Dimethyl-benzyl)-6,7-dihydro-5H-pyrrolo[3,4-d]pyrimidin-4-yl]-quinolin-3-yl-amine;
[6-(2,5-Dimethyl-benzyl)-6,7-dihydro-5H-pyrrolo[3,4-d]pyrimidin-4-yl]-quinolin-3-yl-amine;
[6-(2-Ethyl-benzyl)-6,7-dihydro-5H-pyrrolo[3,4-d]pyrimidin-4-yl]-quinolin-3-yl-amine;
[6-(2-Chloro-thiazol-5-ylmethyl)-6,7-dihydro-5H-pyrrolo[3,4-d]pyrimidin-4-yl]-quinolin-3-yl-amine;
[6-(2-Methyl-benzyl)-6,7-dihydro-5H-pyrrolo[3,4-d]pyrimidin-4-yl]-quinolin-3-yl-amine;
[6-(2,4-Dimethyl-benzyl)-6,7-dihydro-5H-pyrrolo[3,4-d]pyrimidin-4-yl]-quinolin-3-yl-amine;
(6-Pyridin-4-ylmethyl-6,7-dihydro-5H-pyrrolo[3,4-d]pyrimidin-4-yl)-quinolin-3-yl-amine;
Quinolin-3-yl-[6-(2-trifluoromethyl-benzyl)-6,7-dihydro-5H-pyrrolo[3,4-d]pyrimidin-4-yl]-amine;
[6-(2-Methyl-furan-3-ylmethyl)-6,7-dihydro-5H-pyrrolo[3,4-d]pyrimidin-4-yl]-quinolin-3-yl-amine;
[6-(2-Ethyl-4-methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzyl)-6,7-dihydro-5H-pyrrolo[3,4-d]pyrimidin-4-yl]-amine;
Quinolin-3-yl-[6-(3-trifluoromethyl-benzyl)-6,7-dihydro-5H-pyrrolo[3,4-d]pyrimidin-4-yl]-amine;
[6-(2-Ethoxy-benzyl)-6,7-dihydro-5H-pyrrolo[3,4-d]pyrimidin-4-yl]-quinolin-3-yl-amine;
[6-(2,3-Dihydro-benzofuran-7-ylmethyl)-6,7-dihydro-5H-pyrrolo[3,4-d]pyrimidin-4-yl]-quinolin-3-yl-amine;
[6-(2-Methylsulfanyl-benzyl)-6,7-dihydro-5H-pyrrolo[3,4-d]pyrimidin-4-yl]-quinolin-3-yl-amine;
(6-Benzo[1,3]dioxol-4-ylmethyl-6,7-dihydro-5H-pyrrolo[3,4-d]pyrimidin-4-yl)-quinolin-3-yl-amine;
Quinolin-3-yl-[6-(3-trifluoromethoxy-benzyl)-6,7-dihydro-5H-pyrrolo[3,4-d]pyrimidin-4-yl]-amine;
[6-(2-Difluoromethoxy-benzyl)-6,7-dihydro-5H-pyrrolo[3,4-d]pyrimidin-4-yl]-quinolin-3-yl-amine;
[6-(3-Methoxy-benzyl)-6,7-dihydro-5H-pyrrolo[3,4-d]pyrimidin-4-yl]-quinolin-3-yl-amine;
[6-(2,3-Difluoro-benzyl)-6,7-dihydro-5H-pyrrolo[3,4-d]pyrimidin-4-yl]-quinolin-3-yl-amine;
[6-(2-Propoxy-benzyl)-6,7-dihydro-5H-pyrrolo[3,4-d]pyrimidin-4-yl]-quinolin-3-yl-amine;
[6-(2-Isopropoxy-benzyl)-6,7-dihydro-5H-pyrrolo[3,4-d]pyrimidin-4-yl]-quinolin-3-yl-amine;
[6-(1H-Indol-7-ylmethyl)-6,7-dihydro-5H-pyrrolo[3,4-d]pyrimidin-4-yl]-quinolin-3-yl-amine;
[6-(3-Methyl-benzyl)-6,7-dihydro-5H-pyrrolo[3,4-d]pyrimidin-4-yl]-quinolin-3-yl-amine;
[6-(2-Cyclopropyl-benzyl)-6,7-dihydro-5H-pyrrolo[3,4-d]pyrimidin-4-yl]-quinolin-3-yl-amine;
(6-Benzyl-6,7-dihydro-5H-pyrrolo[3,4-d]pyrimidin-4-yl)-quinolin-3-yl-amine;
(6-Cyclohexylmethyl-6,7-dihydro-5H-pyrrolo[3,4-d]pyrimidin-4-yl)-quinolin-3-yl-amine;
(6-Cyclopentylmethyl-6,7-dihydro-5H-pyrrolo[3,4-d]pyrimidin-4-yl)-quinolin-3-yl-amine;
[6-(2-Ethyl-butyl)-6,7-dihydro-5H-pyrrolo[3,4-d]pyrimidin-4-yl]-quinolin-3-yl-amine;
[6-(2-Methyl-pyridin-3-ylmethyl)-6,7-dihydro-5H-pyrrolo[3,4-d]pyrimidin-4-yl]-quinolin-3-yl-amine;
Quinolin-3-yl-[6-(tetrahydro-thiopyran-4-ylmethyl)-6,7-dihydro-5H-pyrrolo[3,4-d]pyrimidin-4-yl]-amine;
[6-(2-Phenyl-propyl)-6,7-dihydro-5H-pyrrolo[3,4-d]pyrimidin-4-yl]-quinolin-3-yl-amine;
(6-Phenethyl-6,7-dihydro-5H-pyrrolo[3,4-d]pyrimidin-4-yl)-quinolin-3-yl-amine;
[6-(2,4-Dimethyl-thiazol-5-ylmethyl)-6,7-dihydro-5H-pyrrolo[3,4-d]pyrimidin-4-yl]-quinolin-3-yl-amine;
Quinolin-3-yl-[6-(tetrahydro-pyran-4-ylmethyl)-6,7-dihydro-5H-pyrrolo[3,4-d]pyrimidin-4-yl]-amine;
[6-(1-Cyclohexyl-ethyl)-6,7-dihydro-5H-pyrrolo[3,4-d]pyrimidin-4-yl]-quinolin-3-yl-amine;
Quinolin-3-yl-[6-(toluene-2-sulfonyl)-6,7-dihydro-5H-pyrrolo[3,4-d]pyrimidin-4-yl]-amine;
[6-(4-Methyl-thiazol-5-ylmethyl)-6,7-dihydro-5H-pyrrolo[3,4-d]pyrimidin-4-yl]-quinolin-3-yl-amine;
5-[4-(Quinolin-3-ylamino)-5,7-dihydro-pyrrolo[3,4-d]pyrimidine-6-sulfonyl]-furan-2-carboxylic acid methyl ester;
[6-(3-Methyl-pyridin-4-ylmethyl)-6,7-dihydro-5H-pyrrolo[3,4-d]pyrimidin-4-yl]-quinolin-3-yl-amine;
[6-(2-Fluoro-benzyl)-6,7-dihydro-5H-pyrrolo[3,4-d]pyrimidin-4-yl]-quinolin-3-yl-amine;
[6-(2-Methoxy-benzyl)-6,7-dihydro-5H-pyrrolo[3,4-d]pyrimidin-4-yl]-quinolin-3-yl-amine;
[6-(2-Chloro-benzyl)-6,7-dihydro-5H-pyrrolo[3,4-d]pyrimidin-4-yl]-quinolin-3-yl-amine;
[6-(3-Methyl-furan-2-ylmethyl)-6,7-dihydro-5H-pyrrolo[3,4-d]pyrimidin-4-yl]-quinolin-3-yl-amine;
[6-(2,3-Dimethyl-benzyl)-6,7-dihydro-5H-pyrrolo[3,4-d]pyrimidin-4-yl]-quinolin-3-yl-amine;
[6-(2,5-Dimethyl-benzyl)-6,7-dihydro-5H-pyrrolo[3,4-d]pyrimidin-4-yl]-quinolin-3-yl-amine;
[6-(2-Ethyl-benzyl)-6,7-dihydro-5H-pyrrolo[3,4-d]pyrimidin-4-yl]-quinolin-3-yl-amine;
[6-(2-Methyl-benzyl)-6,7-dihydro-5H-pyrrolo[3,4-d]pyrimidin-4-yl]-quinolin-3-yl-amine;
(6-Pyridin-4-ylmethyl-6,7-dihydro-5H-pyrrolo[3,4-d]pyrimidin-4-yl)-quinolin-3-yl-amine;
Quinolin-3-yl-[6-(2-trifluoromethyl-benzyl)-6,7-dihydro-5H-pyrrolo[3,4-d]pyrimidin-4-yl]-amine;
[6-(2-Ethyl-4-methyl-thiazol-5-ylmethyl)-6,7-dihydro-5H-pyrrolo[3,4-d]pyrimidin-4-yl]-quinolin-3-yl-amine;
Quinolin-3-yl-[6-(2-trifluoromethoxy-benzyl)-6,7-dihydro-5H-pyrrolo[3,4-d]pyrimidin-4-yl]-amine;
Quinolin-3-yl-[6-(3-trifluoromethyl-benzyl)-6,7-dihydro-5H-pyrrolo[3,4-d]pyrimidin-4-yl]-amine;
[6-(2-Ethoxy-benzyl)-6,7-dihydro-5H-pyrrolo[3,4-d]pyrimidin-4-yl]-quinolin-3-yl-amine;
[6-(2,3-Dihydro-benzofuran-7-ylmethyl)-6,7-dihydro-5H-pyrrolo[3,4-d]pyrimidin-4-yl]-quinolin-3-yl-amine;
[6-(2-Methylsulfanyl-benzyl)-6,7-dihydro-5H-pyrrolo[3,4-d]pyrimidin-4-yl]-quinolin-3-yl-amine;
(6-Benzo[1,3]dioxol-4-ylmethyl-6,7-dihydro-5H-pyrrolo[3,4-d]pyrimidin-4-yl)-quinolin-3-yl-amine;
Quinolin-3-yl-[6-(3-trifluoromethoxy-benzyl)-6,7-dihydro-5H-pyrrolo[3,4-d]pyrimidin-4-yl]-amine;
[6-(2-Difluoromethoxy-benzyl)-6,7-dihydro-5H-pyrrolo[3,4-d]pyrimidin-4-yl]-quinolin-3-yl-amine;
[6-(3-Methoxy-benzyl)-6,7-dihydro-5H-pyrrolo[3,4-d]pyrimidin-4-yl]-quinolin-3-yl-amine;
[6-(2,3-Difluoro-benzyl)-6,7-dihydro-5H-pyrrolo[3,4-d]pyrimidin-4-yl]-quinolin-3-yl-amine;
[6-(1H-Indol-7-ylmethyl)-6,7-dihydro-5H-pyrrolo[3,4-d]pyrimidin-4-yl]-quinolin-3-yl-amine;
[6-(3-Methyl-benzyl)-6,7-dihydro-5H-pyrrolo[3,4-d]pyrimidin-4-yl]-quinolin-3-yl-amine;
[6-(2-Cyclopropyl-benzyl)-6,7-dihydro-5H-pyrrolo[3,4-d]pyrimidin-4-yl]-quinolin-3-yl-amine; and
[6-(3-Methyl-butyl)-6,7-dihydro-5H-pyrrolo[3,4-d]pyrimidin-4-yl]-quinolin-3-yl-amine;
or a pharmaceutically acceptable salt, solvate or prodrug thereof;
and stereoisomers, isotopic variants and tautomers thereof.

51. (canceled)

52. A pharmaceutical composition comprising a pharmaceutically acceptable carrier and a pharmaceutically effective amount of a compound of claim 1.

53. (canceled)

54. (canceled)

55. (canceled)

56. A method for preventing, treating or ameliorating in a mammal a disease or condition associated with the aberrant behavior of FAAH in vivo or which can be alleviated by modulating FAAH activity, which comprises administering to the mammal an effective disease-treating or condition-treating amount of a compound according to claim 1.

57. The method of claim 56, wherein the disease or condition is selected from: pain including acute, inflammatory and neuropathic pain, chronic pain, dental pain and headache including migraine, cluster headache and tension headache, Parkinson's disease, Alzheimer's disease and multiple sclerosis; diseases and disorders which are mediated by or result in inflammation, arthritis, rheumatoid arthritis and osteoarthritis, and inflammatory bowel disease; diseases and disorders which are mediated by or result in neuroinflammation, encephalitis; centrally-mediated neuropsychiatric diseases and disorders, depression mania, bipolar disease, anxiety, schizophrenia, weight and eating disorders, sleep disorders and cognition disorders; neurological and neurodegenerative diseases and disorders; epilepsy and seizure disorders; addiction, spasticity, glaucoma, respiratory and airway disease and disorders, allergic rhinitis, asthma and reactive airway disease and chronic obstructive pulmonary disease; myocardial infarction, autoimmune diseases and disorders, itch/pruritus, psoriasis; obesity; lipid disorders; hypertension; emesis; nausea; cancer; and renal disorders.

58. (canceled)

59. (canceled)

60. (canceled)

61. (canceled)

62. (canceled)

63. (canceled)

64. (canceled)

65. (canceled)

66. (canceled)

67. (canceled)

68. A combination of a compound as defined in claim 1, and another pharmacologically active agent.

69. (canceled)

70. (canceled)

71. (canceled)

72. A method for preventing, treating or ameliorating in a mammal a disease or condition associated with the aberrant behavior of FAAH in vivo or which can be alleviated by modulating FAAH activity, which comprises administering to the mammal an effective disease-treating or condition-treating amount of a pharmaceutical composition according to claim 52.

73. The method of claim 72, wherein the disease or condition is selected from: pain including acute, inflammatory and neuropathic pain, chronic pain, dental pain and headache including migraine, cluster headache and tension headache, Parkinson's disease, Alzheimer's disease and multiple sclerosis; diseases and disorders which are mediated by or result in inflammation, arthritis, rheumatoid arthritis and osteoarthritis, and inflammatory bowel disease; diseases and disorders which are mediated by or result in neuroinflammation, encephalitis; centrally-mediated neuropsychiatric diseases and disorders, depression mania, bipolar disease, anxiety, schizophrenia, weight and eating disorders, sleep disorders and cognition disorders; neurological and neurodegenerative diseases and disorders; epilepsy and seizure disorders; addiction, spasticity, glaucoma, respiratory and airway disease and disorders, allergic rhinitis, asthma and reactive airway disease and chronic obstructive pulmonary disease; myocardial infarction, autoimmune diseases and disorders, itch/pruritus, psoriasis; obesity; lipid disorders; hypertension; emesis; nausea; cancer; and renal disorders.
